SEQUENCE LISTING (17 sequences)

SEQ ID NO: 1 - DNA, 17 bp, Artificial Sequence
  Synthetic primer
gcgggcggcggctattg

SEQ ID NO: 2 - DNA, 20 bp, Artificial Sequence
  Synthetic primer
gccgacaggatcgaaaatcg

SEQ ID NO: 3 - DNA, 33 bp, Artificial Sequence
  Synthetic primer
gcattgatctagaatggagaaatgggcaagaat

SEQ ID NO: 4 - DNA, 33 bp, Artificial Sequence
  Synthetic primer
aataatagtcgacagcggctttgagcttagtcg

SEQ ID NO: 5 - DNA, 18 bp, Artificial Sequence
  Synthetic primer
tggcctccaaccagaacc

SEQ ID NO: 6 - DNA, 20 bp, Artificial Sequence
  Synthetic primer
acgactaaaggaacggaaat

SEQ ID NO: 7 - DNA, 20 bp, Artificial Sequence
  Synthetic primer
cgataacctggtcaagatcc

SEQ ID NO: 8 - DNA, 20 bp, Artificial Sequence
  Synthetic primer
ctgctcccacatgatgatta

SEQ ID NO: 9 - DNA, 20 bp, Artificial Sequence
  Synthetic primer
ctgcccgtaccatcacctac

SEQ ID NO: 10 - DNA, 20 bp, Artificial Sequence
  Synthetic primer
gtgatctgggcgttaccagt

SEQ ID NO: 11 - DNA, 20 bp, Artificial Sequence
  Synthetic primer
tcttgatggtgctgcttacg

SEQ ID NO: 12 - DNA, 20 bp, Artificial Sequence
  Synthetic primer
ccaaactcagcttcctcagc

SEQ ID NO: 13 - DNA, 20 bp, Artificial Sequence
  Synthetic primer
cgtaagcagcaccatcaaga

SEQ ID NO: 14 - DNA, 2493 bp, Butyrivibrio fibrisolvens
atggagaaatgggcaagaatcaaatatacaccaaatcttccgcttggagagaatggtgaaagggttacagcgagtcagaagcacattgagctttcatgcgaggcagcatgtgagggaatggtacttctcaagaatgacagaaacgttcttcctatcagaaagggcacaagagtagccctctttggaaagggagtatttgactatgtaaaaggcggcggtggtagcggagatgtaacagttccttacatcagaaacctctacgaaggcctttctcagtacacatcagacatttcaatttacgacaaatctgtcagattctatcaggaatatgtagcagaccagtacagacttggaattgcaccaggcatgatcaaagagccggctcttccggaagatattcttgcagatgcagcagcctatgcagatactgcaatcatcgcaatcagcagattctccggagaaggctgggacagaaaggttgcaggcgttgacagagaaatcaagtgcgaagccaaggacctcgtagagcagggcaacaagatatttgatcatggtgatttctacctcacaaatgctgagaagaagatggtcaagatggtaaaagagaacttctcaagcgtcattgtagtcatgaatgtcggaggagtcgtagacacaacatggtttaaaaaggatgaccagatttcatcagtcctcatggcatggcagggtggaattgaaggcggacttgccgcagccaggatccttcttggcaaggttaatccttcaggtaagctctcagatacattcgcagcaaggcttgaagactatccttcaacagagggcttccacgaagatgatgactacgtggattacacagaagatatctacgttggctatagatatttcgagaccattcccggggcaaaagagaaagttaactacccctttggctatggcctttcctatacaactttcctgcttgaagactataaggcagagccttttgtggcttcagcagcagacgaggtcggtaaatctgatagcgaccttgcagatgcaatcgtagcctcagttacagtcacaaacattggcaagattccgggcaaagaggttgttcagctctactacagcgctcctcagggcaagctcggtaagcctgctaaagtccttggcggctatgccaagacaaggctactgcagccgggagagagccagagagtgacaattgctctttatatggaggatatggcatcttacgacgaccttggcaaggttaaaaaggctgcctggctccttgaaaaaggtgaatatcatttcttccttggaacatcagtaagagacacaaggcttcttgattacacctatgaactttctaagaacataatagttgaacaggtctcaaacaagctcgttccaacatctcttcccaagagaatgcttgctgatggcacatatgaggaacttcctcagacagaacctgtagatacttatgcaacaatcttcccaagacctaagaactggaaagaaacaattgagcacgacgtattaaagactcctgtagttcgtccacaggacagattccagctctttttgccacctaaggaaggtgaccctaagaaatttatcgaagttgcagaatgcaaggtgacacttgaagactttattgcacagctatctaacgagcagcttgcaagccttcttggaggacagccaaatgtcggaatggctaacacctttggatacggcaaccttcctgaggttggagttcctaatgcccagacctgtgatggtcctgcaggtgtccgtattgcaccggaagttggtgttgtgactacagcattcccatgttcaacccttcttgcatgcacatggaatgaagatatctgctacgaagtcggagttgcaggcggagaagaggccaaggagtgcaattttggtgcatggcttactcctgctgttaacatccatagaagccctctttgcggcagaaactttgagtactactccgaagatccattccttgcaggtaaacaggcagcagctatggttcgtggtatccagagcaacaacataattgctacacctaaacattttgccctcaacaacaaggaatccaatagaaaaggcagcgattcacgtgcttctgagcgtgcgatcagagaaatatatttaaaggcctttgaaatcattgttaaagagcagagccctggagcatcatgtcttcaatacaatatagttaacggtcagagatcatccgaatctcacgacctcctcacaggaatcctccgcgatgagtggggctttgaaggtgttgtagtcagcgactggtggggctttggtgagcattacaaggaagtccttgcaggcaacgatatcaagatgggctgtggctatacagaacagctccttgaagcaattgataagaaagctcttaagagaaaagatttggaaaagaggcagagcgagtcctcaagatgcttctcaaactcgactaagctcaaagccgcttag

SEQ ID NO: 15 - protein, 830 aa, Butyrivibrio fibrisolvens
MEKWARIKYTPNLPLGENGERVTASQKHIELSCEAACEGMVLLKNDRNVLPIRKGTRVALFGKGVFDYVKGGGGSGDVTVPYIRNLYEGLSQYTSDISIYDKSVRFYQEYVADQYRLGIAPGMIKEPALPEDILADAAAYADTAIIAISRFSGEGWDRKVAGVDREIKCEAKDLVEQGNKIFDHGDFYLTNAEKKMVKMVKENFSSVIVVMNVGGVVDTTWFKKDDQISSVLMAWQGGIEGGLAAARILLGKVNPSGKLSDTFAARLEDYPSTEGFHEDDDYVDYTEDIYVGYRYFETIPGAKEKVNYPFGYGLSYTTFLLEDYKAEPFVASAADEVGKSDSDLADAIVASVTVTNIGKIPGKEVVQLYYSAPQGKLGKPAKVLGGYAKTRLLQPGESQRVTIALYMEDMASYDDLGKVKKAAWLLEKGEYHFFLGTSVRDTRLLDYTYELSKNIIVEQVSNKLVPTSLPKRMLADGTYEELPQTEPVDTYATIFPRPKNWKETIEHDVLKTPVVRPQDRFQLFLPPKEGDPKKFIEVAECKVTLEDFIAQLSNEQLASLLGGQPNVGMANTFGYGNLPEVGVPNAQTCDGPAGVRIAPEVGVVTTAFPCSTLLACTWNEDICYEVGVAGGEEAKECNFGAWLTPAVNIHRSPLCGRNFEYYSEDPFLAGKQAAAMVRGIQSNNIIATPKHFALNNKESNRKGSDSRASERAIREIYLKAFEIIVKEQSPGASCLQYNIVNGQRSSESHDLLTGILRDEWGFEGVVVSDWWGFGEHYKEVLAGNDIKMGCGYTEQLLEAIDKKALKRKDLEKRQSESSRCFSNSTKLKAA

SEQ ID NO: 16 - DNA, 2031 bp, Cochliobolus carbonum
caccatgctatgcggcgtatggtaacatctgcctcagttgtcggcgtcgctactctggcggccgctaccgtccttatcacaggaggaatcgcccatgccgcatctactctaaagcagggagccgaagctaatggaagatattttggagtctcagcttccgtgaatacccttaacaatagtgccgcagcaaatcttgtcgcaacccaattcgacatgcttaccccagagaacgagatgaaatgggatacagtggaaagctccagaggttcctttaatttcgggccaggagatcaaatcgttgcatttgctacagcccataatatgcgcgttagagggcataatctggtatggcattctcaacttccaggatgggtctcgtcacttccactgagccaggtgcagtctgctatggagagtcatatcacagcagaggtcacgcactacaagggcaaaatatacgcatgggacgttgtgaacgagccttttgatgactctggtaaccttcgtacagatgttttctaccaggcaatgggtgcagggtacatcgctgacgctctgcgaactgcgcatgctgctgacccaaacgcaaagctctaccttaacgactataatattgagggtattaacgctaaaagtgacgctatgtacaacctcatcaaacaacttaagtcacagggagtccctattgatggcgtaggattcgaaagccacttcattgtgggccaagtgccctccacactccaacagaatatgcagcgttttgctgatctcggagtcgatgttgccataacagaattagatgacaggatgcctactccgccttcccaacaaaaccttaatcaacaggccaccgatgatgctaacgtggtaaaagcttgcttggcggttgctcgatgtgtaggaattacacagtgggatgtaagcgacgcagattcttgggttcctggcaccttctcaggtcagggcgccgcaactatgtttgatagcaatttacaaccaaagcctgctttcactgccgtcttgaacgcgctttctgcatccgcctctgtatcaccttctccgtccccgtcaccctccccttctccaagcccatctccgtcaccatcaccttcacctagcccatcaccatctccatcaccttccccttcctcttctccagtcagtggcggtgttaaagttcaatataagaataacgattccgctccgggagacaaccagataaaacctggcctgcaagtagttaatactggatcgtcatcggttgatctgagtacggttaccgttagatactggttcacacgagacggaggatcttccacattggtttacaattgcgactgggctgttatgggttgtggtaatattcgcgctagctttggatcagttaatccagcaacccctacggctgatacgtatctacaactctccttcacaggaggcacactacccgcaggaggatcaaccggtgagatacagtccagggttaacaaaagcgactggtcaaacttcactgagaccaatgactacagctatggtactaacaccaccttccaagattggtcaaaggtcactgtatacgttaatggtcgattggtctggggcaccgaaccttccggaacatccccctctccaacaccatctcctagtccaaccccatctccaagcccttccccatctcccagtccttcaccttcgccatccccctcaccatccccatctccgtcttcttctccgagttcgggctgcgtggcatctatgagagttgattctagctggcctggcggttttacagccactgttactgtgtccaacactggaggcgtctctacttcaggctggcaagttggctggagctggccttcaggcgatagtttagtaaacgcgtggaatgcagttgtttctgttacaggtaccagtgttagagctgtgaatgcaagctataatggagtgatccccgcaggaggatcaaccactttcggtttccaggcgaatggcacaccaggcacaccgacgtttacttgtactacctca

SEQ ID NO: 17 - protein, 677 aa, Cochliobolus carbonum
HHAMRRMVTSASVVGVATLAAATVLITGGIAHAASTLKQGAEANGRYFGVSASVNTLNNSAAANLVATQFDMLTPENEMKWDTVESSRGSFNFGPGDQIVAFATAHNMRVRGHNLVWHSQLPGWVSSLPLSQVQSAMESHITAEVTHYKGKIYAWDVVNEPFDDSGNLRTDVFYQAMGAGYIADALRTAHAADPNAKLYLNDYNIEGINAKSDAMYNLIKQLKSQGVPIDGVGFESHFIVGQVPSTLQQNMQRFADLGVDVAITELDDRMPTPPSQQNLNQQATDDANVVKACLAVARCVGITQWDVSDADSWVPGTFSGQGAATMFDSNLQPKPAFTAVLNALSASASVSPSPSPSPSPSPSPSPSPSPSPSPSPSPSPSPSSSPVSGGVKVQYKNNDSAPGDNQIKPGLQVVNTGSSSVDLSTVTVRYWFTRDGGSSTLVYNCDWAVMGCGNIRASFGSVNPATPTADTYLQLSFTGGTLPAGGSTGEIQSRVNKSDWSNFTETNDYSYGTNTTFQDWSKVTVYVNGRLVWGTEPSGTSPSPTPSPSPTPSPSPSPSPSPSPSPSPSPSPSPSSSPSSGCVASMRVDSSWPGGFTATVTVSNTGGVSTSGWQVGWSWPSGDSLVNAWNAVVSVTGTSVRAVNASYNGVIPAGGSTTFGFQANGTPGTPTFTCTTS

(19) United States
(12) Patent Application Publication
(10) Pub. No.: US 2009/0203079 A1
(43) Pub. Date: Aug. 13, 2009

(54) Transgenic monocot plants encoding beta-glucosidase and xylanase

(75) Inventors: Masomeh B. Sticklen, East Lansing, MI (US); Callista B. Ransom, Lansing, MI (US)

Correspondence Address: Ian C. McLeod;IAN C. McLEOD, P.C., 2190 Commons Parkway, Okemos, MI, 48864, US

(73) Assignee: Board of Trustees of Michigan State University, East Lansing, MI (US)

(21) Appl. No.: 12/383,941

(22) Filed: Mar. 30, 2009

Related U.S. Application Data
(63) Continuation-in-part of application No. 11/489,234, filed on Jul. 19, 2006.
Application No. 11/489,234 is a continuation-in-part of application No. 11/451,162, filed on Jun. 12, 2006, now abandoned.
Application No. 11/489,234 is a continuation-in-part of application No. 09/981,900, filed on Oct. 18, 2001, now Pat. No. 7,049,485.
Application No. 11/451,162 is a continuation-in-part of application No. 09/981,900, filed on Oct. 18, 2001, now Pat. No. 7,049,485.
(60) Provisional application No. 61/072,893, filed on Apr. 2, 2008.
(60) Provisional application No. 60/242,408, filed on Oct. 20, 2000.

(51) Int. Cl. C12P 19/00; C12N 9/24; C12N 9/42; C12P 7/08

(52) U.S. Cl. 435/72; 435/200; 435/209; 435/163

(57) ABSTRACT

Plant proteins isolated from monocot plants from transformation of the monocot plant with DNA at least 80% homologous to the bglA gene encoding β-glucosidase from a rumen bacterium which is Butyrivibrio fibrisolvens H17c and targeted to a subcellular compartment. The transformed plant is ground after the β-glucosidase has been accumulated, and the protein is extracted or used directly with the ground plant material to degrade cellobiose, in particular, to produce sugars used in fermentations, particularly to produce ethanol. Also, a gene at least 80% homologous to DNA XYL1 gene encoding a xylanase is also provided in a transformed plant and used to produce sugars.

### CROSS-REFERENCE TO RELATED APPLICATIONS

This application claims benefit to U.S. Provisional Application Ser. No. 61/072,893, filed Apr. 2, 2008. This application is a continuation-in-part of both application Ser. No. 11/489,234, filed Jul. 19, 2006 and application Ser. No. 11/451,162, filed Jun. 12, 2006. application Ser. Nos. 11/489,234 and 11/451,162 are continuations-in-part of application Ser. No. 09/981,900, filed Oct. 18, 2001 (now U.S. Pat. No. 7,049,485, which issued on May 23, 2006). U.S. Pat. No. 7,049,485 claims priority to Provisional Application Ser. No. 60/242,408, filed Oct. 20, 2000. These applications and patent are incorporated herein by reference in their entirety.

### STATEMENT REGARDING FEDERALLY SPONSORED RESEARCH OR DEVELOPMENT

Not Applicable.

### REFERENCE TO A “COMPUTER LISTING APPENDIX SUBMITTED ON A COMPACT DISC”

The application contains seventeen (17) sequences which are identified with SEQ ID NOS. A compact disc is provided which contains the Sequence Listings for the sequences. The Sequence Listing on the compact disc is identical to the paper copy of the Sequence Listing provided with the application.

### BACKGROUND OF THE INVENTION

(1) Field of the Invention

The present invention relates to transformed monocot plants with a gene encoding β-glucosidase and/or xylanase in a subcellular compartment. In particular, the gene is at least 80% homologous to the bglA gene from a rumen bacterium Butyrivibrio fibrisolvens H17c. The xylanase is at least 80% homologous to XYL1 gene.

(2) Description of the Related Art

The U.S. needs a competitive substitute for fossil fuels; ethanol biofuel is an attractive choice in a suite of alternatives. Production of ethanol involves fermentation of sugars, which can come from plants as simple sugars, starches, or complex structural polysaccharides of the plant cell wall.

Ethanol production from plant biomass requires pretreatment to disrupt the lignin; addition of the hydrolysis enzymes to the pretreated feedstock; and fermentation of the resulting sugars to ethanol, which must then be distilled. The enzymes necessary for cell wall degradation include cellulases (endo- and exo-glucanases and β-glucosidases), and hemicellulases (most importantly xylanases). Roadblocks stand in the way of this technology becoming mature and economically feasible, including the high costs of enzymes and pretreatment.

Ethanol fuel is a promising alternative to fossil fuels, which damage the environment by contributing to net carbon dioxide increase. In addition, they will eventually be depleted, and increase dependence on foreign oil imports. According to a recent report from the Natural Resources Defense Council and the Institute for the Analysis of Global Security, the dependence of the United States on foreign petroleum both undermines its economic strength and threatens its national security (Bordetsky et al. 2005). The use of ethanol fuel, obtained either from grain or from cellulosic materials, can help decrease the need for petroleum fuel (Bordetsky et al. 2005). Accordingly, the ethanol fuel industry has been growing significantly in many countries throughout the world. In the US, ethanol production capacity reached 3.5 billion gallons in 2004, up by 303 million gallons from 2003 (Renewable Fuels Association 2007). Ethanol fuel is clean-burning and does not contribute to net carbon dioxide increase, is renewable, and can be produced using resources the country already possesses.

Ethanol is produced from the fermentation of sugars (usually sucrose or glucose) by yeast. The carbon (sugar) source is called the feedstock. Most feedstocks are plant materials. The most widely used feedstocks today are sugarcane and maize grain. The sugar in sugarcane is easily extracted and used directly for fermentation, while the maize grain must be milled and its starch hydrolyzed to glucose by α-amylase. In the US, ethanol is mostly produced from the starch of maize grain with a net energy balance of 1.34; that is, for every unit of energy expended in growing corn and converting it to ethanol, 1.34 units of energy (automotive fuel) are obtained (Biomass Program: Net Energy Balance for Bioethanol Production and Use; Shapouri et al. 2002). The most efficient farming and ethanol production systems in place can achieve a balance of 2.09 (Biomass Program: Net Energy Balance for Bioethanol Production and Use). Starch fermentation is thus relatively efficient. However, there is a very rich source of glucose that has so far been underutilized: cellulose.

Cellulose, composed of β-glucose units, is the most abundant polymer on earth. It is a structural component of the plant cell wall. It has traditionally not been used as a carbon source because its location inside microfibrils, which are wrapped in hemicellulose and embedded in a matrix of lignin, makes it inaccessible to hydrolysis enzymes unless the plant material goes through extensive pretreatment. However, recent advancements have made using this resource a possibility. In this chapter, we explore the problems, challenges, and solutions to ethanol production from cellulosic materials, with a focus on utilizing plants as biofactories for hydrolysis enzyme production.

### 2. The Plant Cell Wall

The plant cell wall is a highly organized structural component composed of a myriad of different polysaccharides, proteins, aromatic substances and other compounds. It has several important functions: it provides structure to the cell, thus determining its shape and even function; it aids in defense against invading pathogens; and it contains signaling molecules that can alert the cell to various environmental stimuli, including pathogenic attack (Carpita and McCann 2002). It is a dynamic structure, and its configuration and composition can vary by plant species, age, tissue, cell types and even within cell wall layers (Ding and Himmel 2006; Bothast and Schlicher 2005). The primary cell wall is formed first from the cell plate during cell division and forms the outside of the cell. Between primary cell walls of adjacent cells is the middle lamella. Secondary cell wall synthesis, if present, usually begins after the primary cell wall has stopped growing, being deposited on the interior of the primary cell wall, often in layers (Carpita and McCann 2002).

Polysaccharides are the primary constituents of the cell wall and form its main structural scaffold. They are composed of long chains of sugar molecules that are covalently linked at various positions and may have side chains. They are made up of various combinations of the 11 monosaccharide sugars commonly found in plant cell walls: glucose (from which all the others are derived), rhamnose, galactose, galacturonic acid, glucouronic acid apiose, xylose, arabinose, mannose, mannuronic acid and fucose (Carpita and McCann 2002).

### 2.1 Cell Wall Components

### 2.1.1. Cellulose

Cellulose is a long, unbranched polymer of up to 15,000 molecules of anhydrous glucose. The glucose molecules are arranged in β-1,4 linkages, which means that each unit is orientated 180° relative to the unit it is attached to. In other words, cellulose is composed of cellobiose units (diglucose molecules connected via β-1,4 linkages). Cellulose is an important polysaccharide found in the primary and secondary cell walls in the form of microfibrils. It makes up 15-30% of the dry mass of primary cell walls and up to 40% of secondary cell walls. The cellulose chains in microfibrils are lined up parallel to each other and consist of crystalline regions, where the cellulose molecules are tightly packed, and amorphous (also called soluble) regions, where the arrangement is less compact. The amorphous regions are staggered so that the overall structure remains strong. A microfibril has a diameter of around 30 nm and consists of around 36 cellulose chains, but the number varies with species (Carpita and McCann 2002).

### 2.1.2 Cross-Linking Glycans

Microfibrils are coated with other polysaccharides, cross-linking glycans (also called hemicelluloses), which link them together. The two major types are xyloglucans, found in dicots and around half of the monocot species, and glucuronoarabinoxylans, which are found in commelinoid monocots, including the cereals and grasses. Xyloglucans have a backbone of glucosyl residues in 1,4-β linkages, with xylosyl units attached; glucuronoarabinoxylans have a backbone of xylosyl residues in 1,4-β linkages to which glucosyluronic acid and arabinosyl units are attached. The grasses also have a third major cross-linking glycan, called “mixed linkage” (1→3), (1→4) β-D-glucans (β-glucans), which are unbranched polymers with a 2:1 ratio of cellotriose to cellotetraose units connected by (1→3)β-D-linkages, resulting in a coiled shape. Various mannans are also present in smaller amounts (Carpita and McCann 2002). Hemicellulose accounts for 20-40% of the total dry weight of plant matter.

### 2.1.3 Pectins and Other Substances

Pectins are a mixed group of various branched, hydrated polysaccharides abundant in galacturonic acid. In dicots, they account for approximately 35% of the dry weight (Carpita and Gibeaut 1993); in monocots they are much less abundant. They serve many functions in the cell wall: they establish wall porosity, adjust wall pH and ion balance through charged surfaces, control bonding between cells at the middle lamella, and also function as recognition molecules to alert the cell to the presence of microorganisms or insects (Ridley et al. 2001; O'Neill et al. 2004). Pectins are mostly made up of homogalacturonan and rhamnogalacturonan I; rhamnogalacturonan II, arabinans, galactans, and arabinogalactans are also present in smaller quantities. In addition to pectins, structural proteins and aromatic substances can also be present (Carpita and McCann 2002).

### 2.1.4 Lignin

Lignin is almost nonexistent in primary cell walls but is a chief constituent in some secondary walls, and accounts for about 10-25% of the total dry weight. It is composed of aromatic compounds called phenylpropanoids arranged in complex systems. These networks are linked to the carbohydrates, including cellulose and xylose, in various bonds, including ester, ester; phenyl, phenyl; and covalent bonds (Carpita and McCann 2002). Lignin protects the cell against pathogen invasion and will often be deposited in response to attack, providing additional structure and strength (Mosier et al. 2005).

### 2.2 Two Major Types of Primary Cell Wall

The basic structure of primary cell walls consists of the scaffold of cellulose and cross-linking glycans, embedded in a second (and sometimes third) complex. There are two types of primary cell wall that differ in the kind of cross-linking glycan, which determines the wall type. Type I walls are found in those plants that have xyloglucans; they have approximately equal amounts of xyloglucan and cellulose. Xyloglucans coat the cellulose microfibrils and bind them together, and this complex is embedded in a matrix of pectin. Type II walls are found in plants whose major cross-linking glycans are glucuronoarabinoxylans; they lack pectin and structural proteins, instead amassing phenylpropanoids (Carpita and McCann 2002). Type II cell walls are found in cereals and grasses, and thus are of greatest interest for cellulosic ethanol research.

### 3. Cell Wall Degradation

### 3.1 Microorganisms

Several microorganisms (bacteria and fungi) have been studied for their ability to break down cell walls, including anaerobes (such as those present in the rumen) and aerobes (such as those that decompose dead plant matter). Most organisms that can degrade cellulose produce a number of enzymes, which form a system that hydrolyzes various polysaccharides, since the enzymes first have to penetrate the hemicellulose shield before they can attack the cellulose (Warren 1996).

Anaerobic microorganisms known for their cell-wall degrading ability include the bacteria Butyrivibrio fibrisolvens H17c, Fibrobacter succinogenes S85, Ruminococcus flavefaciens 17, R. albus, Prevotella ruminicola B14, Clostridium thermocellum, C. cellulovorans, C. cellulolyticum, C. stercorarium and Caldocellulosiruptor saccharolyticus, and the fungus Neocallimastix frontalis. Aerobic microorganisms include the bacteria Acidothermus cellulolyticus (Tucker et al. 1989), Pseudomonas fluorescens subsp. cellulosa, Streptomyces lividans 66, S. reticuli, S. halstedii, Cellulomonas fimi, C. uda and Microbispora bispora, and the fungi Thermomonospora fusca, Trichoderma reesei and Phanerochaete chrysosporium (Warren 1996).

These organisms produce many different enzymes that may be grouped according to their primary activities: endoglucanases, exoglucanases (also called cellobiohydrolyases), β-glucosidases, cellodextrinases, xylanases, xylosidases, lichenases, mannanases, laminarinases, arabinofuranosidases and avicelases. In order to decrystallize and hydrolyze cell walls, they must produce systems of many different enzymes (for each of the cell wall components) that act synergistically; this has been well documented. The enzymes vary in their substrate specificity: some exclusively act on a particular substrate, while others can utilize more than one; some have more activity on one substrate over another; and some can break only certain bonds, while others can cleave more than one bond type. In addition, different enzymes often produce different products from the same substrate. Therefore microorganisms may produce several different enzymes, for specific substrates or bonds or both. Some microorganisms, such as Clostridia spp., produce cellulosomes (Demain et al. 2005), complexes of multiple enzymes held together in a specific conformation by proteins that are very efficient at cell wall hydrolysis (Warren 1996).

### 3.2 Hydrolysis

The major classes of enzymes needed for cell wall hydrolysis are cellulases, hemicellulases and ligninases.

### 3.2.1 Cellulases

Three types of cellulases are needed to obtain glucose from cellulose: endoglucanase (E1; E.C. 3.2.1.4), cellobiohydrolase (also called exoglucanase) (E.C. 3.2.1.91), and β-glucosidase (E.C. 3.2.1.21) (Ziegler et al. 2000; Ziegelhoffer et al. 2001). Enzymatic hydrolysis of plant cell wall polysaccharides to glucose is a three-step process. First, endoglucanase randomly cleaves the crystalline regions of cellulose, exposing chain ends. Then, cellobiohydrolase attaches to the chain end and threads it through its active site, processively cleaving off cellobiose units; it can also act on amorphous regions with exposed chain ends without prior endoglucanase activity. Exoglucanases work from either the reducing or non-reducing end of the sugar, not both; cellulase hydrolysis is more efficient if both types are produced. Finally, β-glucosidase breaks the bonds of cellobiose to produce single glucose units (Warren 1996).

### 3.2.2 Hemicellulases

For cellulases to access the cellulose, the hemicellulose surrounding it must be removed. While cellulose consists of a single monosaccharide and type of bond, hemicelluloses are amorphous and diverse. Since the major constituent of hemicellulose is β-1,4-xylan, the most abundant class of hemicellulase is xylanase, which can have both endo- and exo-activity (Warren 1996).

### 3.2.3 Ligninases

Lignin degradation by microorganisms is less well understood than that of polysaccharides. The most effective lignin-degrading microbes in nature are thought to be white rot fungi (D'Souza et al. 1999), especially Phanerochaete chrysosporium and Trametes versicolor. The three major families of lignin-modifying enzymes produced by fungi are laccases, manganese-dependent peroxidases, and lignin peroxidases (Boominathan and Reddy 1992; Hatakka 1994; Kirk and Farrell 1987; Thurston 1994). They oxidize compounds by using or creating radicals.

### 4. Ethanol Production

### 4.1 Maize Grain Ethanol Production

Ethanol produced from maize grain is a mature technology. It is attractive because it benefits farmers and local communities by providing jobs, a valuable resource, and valuble coproducts (such as distillers grains and corn gluten). As of 2007, 124 biorefineries are in operation and 76 more are being constructed. Ethanol production currently stands at nearly 6.5 billion gallons a year and will reach 12.9 billion gallons per year upon the plants' completion (RFA—The Industry—Plant Locations), which could displace 4.7 and 9.3 billion gallons of gasoline respectively (if E85, a fuel blend of gasoline and up to 85% ethanol, is used). However, this only covers around 3% or 6.7% respectively of the total gasoline consumed annually in the U.S. (137 billion gallons in 2006; (U.S. Prime Supplier Sales Volumes of Petroleum Products).

U.S. maize growers produced 10.5 billion bushels of maize grain in 2006 (World of Corn 2007); 18.3% was used in ethanol production (World of Corn 2007). This is the equivalent of 2.2 billion bushels, or 6.2 billion gallons of ethanol, which likely displaced 4.4 billion gallons of gasoline (3.2% total consumption). Since current ethanol plant capacity is 2.3 billion bushels, becoming 4.6 billion bushels, grain production must increase to meet capacity, or must be diverted from other uses. Currently, 50.8% of total production, or 6 billion bushels, is used for livestock feed (World of Corn 2007). Much of this could be successfully diverted to ethanol fuel production as the grain could be replaced with nutritious distillers grains. To meet capacity, only 1.7% (currently) or 40% (when the plants are completed) need be diverted from grain destined for livestock feed (0.1 billion bushels and 2.4 billion bushels respectively). Therefore meeting production capacity from maize grain is an attainable goal and likely to be realized. However, if all the maize grain produced in the US were used for ethanol fuel production, only 29.4 billion gallons would be produced, the equivalent of 21.2 billion gallons of fuel, or 15.4% of current usage (Houghton et al. 2005). Clearly, an alternative to maize grain ethanol is needed.

### 4.2 The Promise of Cellulosic Ethanol

According to Kim and Dale (2004), worldwide wasted crops and lignocellulosic waste crop residue could translate into 129.7 billion gallons of ethanol and replace 93.4 billion gallons of gasoline (about 32% of current worldwide consumption) if E85 is used. About 90% of this estimate comes from crop residue waste. This number could be much higher if biofuel crops were grown to supplement this amount and if the technology were in place to produce it. Worldwide availability of lignocellulosic feedstocks is estimated at over 1.7 billion tons per year (Kim and Dale 2004), with some estimates reaching 10-50 billion tons of crop biomass annually (Greene et al. 2004). In addition to being inexpensive and widely available, lignocellulosic biomass has the added benefit of being renewable and thus sustainable (Kim and Dale 2004; Greene et al. 2004). It is believed that with proper management, roughly 1.3 billion tons of crop and forest residues and energy crops can become available annually in the US (Perlack et al. 2005), the majority of which could be used for conversion to alcohol fuels, yielding the equivalent of approximately 108.5 billion gallons of gasoline (Kim and Dale 2004).

A current goal for enhancing US economic security is to meet 10% of chemical feedstock demand by 2020 with plant-derived materials, or a fivefold increase over current usage levels (Singh et al. 2003). Crops that have a high amount of lignocellulosic biomass, such as corn, rice, sugarcane and fast growing perennial grasses have been recommended for conversion to alcohol fuels (Knauf and Moniruzzaman 2004; Sticklen 2004).

Construction of commercial cellulosic biomass ethanol facilities is currently underway in the US. These facilities will have the capactiy to collectively produce 226.4 million gallons per year. They include: Abengoa Abengoa Bioenergy, NE; Akico, Inc., FL; Bluefire Ethanol, CA; Broin Companies, IA; Iogen Biorefinery Partners, ID; and Range Fuels, GA (Bruce Dale, Michigan State University, pers. comm.). In Canada, Iogen Corporation has a demonstration biomass ethanol plant currently in operation that can produce about 660,000 gallons of ethanol per year.

### 4.2.1 Cellulosic Ethanol Production

To produce ethanol from biomass, several events must take place: the hydrolysis enzymes must be produced (usually in microbial fermentation tanks), the biomass must undergo a pretreatment process to disrupt the lignin and expose the cellulose, the enzymes must be added to the pretreated feedstock, and the resulting sugars must be fermented and distilled.

### 4.2.2 Challenges to Cellulosic Ethanol Production

Although production of fermentable sugars for alcohol fuels from plant biomass is an exciting and attractive idea, and substantial efforts have been made toward improving ethanol yield through this technology and reducing its production costs (Ingledew 1995; Lynd et al. 2005), major roadblocks still stand in the way of widespread commercial implementation of this technology. These include prohibitive costs of pretreatment processing of the lignocellulosic matter, with estimates of up to $0.30/gallon (Mosier et al. 2005) and production of microbial cellulase enzymes used in the conversion of cellulosic matter to fermentable sugars (Kabel et al. 2006).

Removal of lignin is the major roadblock to this process and an area of intense research because of the high cost involved. Although research is ongoing in the area of fungal ligninases (mentioned above) and reduction of lignin content (described below) in order to decrease the necessity (and thus the cost) of pretreatment, pretreatment is currently required. Several pretreatments have been developed so far, including dilute acid, flow-through, ammonia fiber explosion (AFEX), ammonia recycle percolation, steam water explosion, lime, and organosolv pulping (Eggeman and Elander 2005; Mosier et al. 2005; Wyman et al. 2005b; Wyman et al. 2005a; Pan et al. 2005).

Currently, production of hydrolysis enzymes in microbial fermentation tanks is expensive (Knauf and Moniruzzaman 2004; Howard et al. 2003). Although decades of research have been devoted to reducing microbial production costs, resulting in significant decreases since 1980 (Knauf and Moniruzzaman 2004; Wyman 1999), enzyme production is still costly (Knauf and Moniruzzaman 2004). The latest cost-reduction model designed by the National Renewable Energy Laboratory (NREL) and Genencor is to produce cellulases at around $0.10-$0.20 per gallon of ethanol (Genencor Celebrates Major Progress in the Conversion of Biomass to Ethanol—Genencor a Danisco division). A possible solution to these problems is to use biomass crops as biofactories to produce these enzymes on a large scale.

### 5. Production of Hydrolysis Enzymes in Biomass Crops

### 5.1 Plants as Molecular Biofactories

Plants are already being used successfully for molecular farming (Horn et al. 2004) of enzymes (Hong et al. 2004; Chiang et al. 2005) and other proteins (Liu et al. 2005), carbohydrates (Schulman 2002; Sahrawy et al. 2004), lipids (Qi et al. 2004), polymers such as polyhydroxybutyrate (Bohmert et al. 2002); (Saruul et al. 2002; Zhong et al. 2003) and pharmaceuticals (Howard and Hood 2005). Plant-based production of enzymes has several critical advantages compared to microbial fermentation or bioreactors. For example, plants can use the sun's energy directly, requiring fewer energy inputs. Furthermore, proteins produced in plants generally display correct folding, glycosylation, activity, reduced degradation and increased stability (Horn et al. 2004). In addition, the infrastructure and expertise are already available for plant genetic transformation, growing, harvesting, transporting and processing plant matter (Horn et al. 2004).

The U.S. Government has recently urged the agricultural and petrochemical industries to discover and employ alternatives to fossil fuels to both decrease dependence on foreign oil and promote a cleaner environment. A specific recommendation was to develop technology that would allow production of cellulases and other hydrolysis enzymes in plants (Ragauskas et al. 2006; Sticklen 2007b; Sticklen 2007a; Sticklen 2004; Sticklen 2006), which has the potential to reduce enzyme production costs. Extraction of plant total soluble protein (TSP) from leaves is quick and easy, and could be done at the ethanol production facilities; alternatively, the enzymes could be extracted and lyophilized for inexpensive storage and easy transport.

### 5.2 Successful Plant-Produced Hydrolysis Enzymes

The catalytic domain of the thermostable endo-1,4-β glucanase (E1) of A. cellulolyticus (Tucker et al. 1989; Baker et al. 1994) has been successfully produced in Arabidopsis (Ziegler et al. 2000), tobacco (Ziegelhoffer et al. 2001), rice (Oraby et al. 2007), and maize (Biswas et al. 2006; Ransom et al. 2007). The full-length peptide has been expressed in potato (Dai et al. 2000b) and tobacco (Dai et al. 2000a; Dai et al. 2005; Ziegelhoffer et al. 2001). Expression of the catalytic domain yielded more activity than the full-length enzyme (Ziegelhoffer et al. 2001). The thermostable endoglucanase E2 of Thermomonospora fusca was expressed in tobacco, potato and alfalfa (Ziegelhoffer et al. 1999).

Rice- and maize-produced transgenic TSP containing E1 was able to convert AFEX-treated corn stover to glucose in conversion analyses with the addition of β-glucosidase (Novozyme 188, Sigma). The enzyme retained activity in dry tissue and after several months' storage in the freezer (Oraby et al. 2007; Ransom et al. 2007).

Exoglucanases have also been expressed in plants. The thermostable cellobiohydrolase E3 of Thermomonospora fusca was expressed in tobacco, potato and alfalfa (Ziegelhoffer et al. 1999). Trichoderma reesei cellobiohydrolase I (CBHI) was produced in transgenic tobacco (Dai et al. 1999). Although a low amount of protein was produced in both cases, biological activity has been low as well.

β-glucosidases have been expressed in plants, although traditionally for reasons other than production of enzymes for hydrolysis. Human acid β-glucosidase was successfully expressed in transgenic tobacco seeds for medical purposes (Reggi et al. 2005). Maize β-glucosidase was expressed in tobacco to study cytokinins (Kiran et al. 2006). Butyrivibrio fibrisolvens H17c β-glucosidase was expressed in tobacco to study whether it could effect enhanced immune response through systemic acquired resistance (SAR) (Yao 2004). This work is the first report of heterologous microbial β-glucosidase expressed in a plant for the purpose of obtaining biologically-active enzyme for use in the production of ethanol fuel.

Microbial xylanases have also been produced in plants. A modified xylanase gene (xynA) from the rumen fungus, Neocallimastix patriciarum, was successfully expressed in barley endosperm, retaining activity after desiccation and storage (Patel et al. 2000). A thermostable xylanase from Clostridium thermocellum was expressed in the apoplast of tobacco (Herbers et al. 1995) and the catalytic domain of XynA from the same organism was expressed in both cultured tobacco cells (Kimura et al. 2003a) and rice (Kimura et al. 2003b). The xynB gene of Streptomyces olivaceoviridis A1 was expressed in potato (Solanum tuberosum); its enzyme activity was retained over several generations (Yang et al. 2007). The purpose in this case was to produce xylanase as an additive for animal feed.

### 5.3 TSP Must be Extracted Prior to Pretreatment

It was originally proposed that hydrolysis enzymes be produced in biomass crops such as maize and switchgrass; then the plants could be subjected to pretreatment, and no additional enzymes would need to be added during the conversion because they would be already present. However, it was found that one of the mildest pretreatments available, Ammonia Fiber Explosion (AFEX), reduced the activity of E1 by about two-thirds (Teymouri et al. 2004), and this approach was abandoned. The new idea is to grow the enzymes in biomass crops, which have the potential to produce large amounts of protein due to a great amount of plant tissue, then extract the TSP and use it in the conversion step. Any plant matter (rice straw, switchgrass, wheat straw, etc.) can be used in conversion, including the plants used for growing the enzymes, after undergoing a pretreatment process.

### 5.4 Thermostable Enzymes are Desirable

Thermostable enzymes from thermophilic microbes are usually stable at high temperature and pH (Bruins et al. 2001) and are thus favorable in industry applications requiring high temperatures to diminish contamination by unwanted microbes. They have the additional advantage of being less active at ambient temperatures and becoming activated upon heating; in the case of cell-wall-degrading enzymes, controlling the activity is important for enzyme production in plants to avoid degradation of the plant before extraction.

### 5.5 Subcellular Targeting and Sequestration

Proteins can be targeted to various subcellular compartments, such as the endoplasmic reticulum (ER), chloroplast, vacuole, or mitochondria, with the use of targeting and/or retention signal peptides. These compartments house various environments that make them desirable for expression of different proteins. Subcellular localization also plays a crucial role in affecting the output of foreign proteins by controlling the interconnected processes of folding, assembly and post-translational modification.

Experiments have compared the targeting of antibodies to the secretory pathway rather than the cytosol and have shown it to be generally more advantageous and superior for folding, assembly and high-level accumulation (Schillberg et al. 1999). In the secretory pathway, proteins first accumulate in the ER and those without a retention signal (H/KDEL carboxy-terminal tetrapeptide tag; Conrad and Fiedler 1998); are secreted to the apoplast. The ER supplies an oxidizing environment and a profusion of molecular chaperones, with few proteases (Fischer et al. 2004). These qualities are probably the most critical influences on protein folding and assembly. In particular, the molecular chaperone BiP has recently been shown to interact specifically with antibodies in transgenic plants that are targeted to the secretory pathway (Nuttall et al. 2002). Proteins are less stable when they are secreted rather than retained in the lumen of the ER; therefore higher accumulation is possible, generally 2-10 fold greater (Schillberg et al. 2003).

It would be strategic to confine the enzymes to different cellular compartments or grow separate enzymes in separate plants. During TSP extraction of enzymes confined to separate cellular compartments, the enzymes would be mixed; if grown in separate plants, the TSP of different plants could be combined before the conversion step. Physical separation of the enzymes will also help avoid enzyme activation until required, as multiple enzymes are needed for synergy to complete hydrolysis, as explained above. Also, if the enzymes are sequestered in various compartments they will not have access to their substrate. In the same vein, if the heterologous protein is toxic to the plant, for example, as avidin produced in the cytosol of tobacco plants, targeting to an organelle (in this case, the vacuole) can allow its accumulation without damaging the host cell (Murray et al. 2002). Several microbial hydrolysis enzymes have already been successfully expressed in plants with subcellular targeting (Table 1).

TABLE 1
Subcellular targeting of various hydrolysis
enzymes.

| Gene | Plant | Target | Reference |
|---|---|---|---|
| E1-_{CAT} | Arabidopsis | Apoplast | Ziegler et al. 2000 |
| E1-_{CAT} | Tobacco | Apoplast | Ziegelhoffer et al. |
| | | | 2001 |
| E1-_{CAT} | Rice | Apoplast | Oraby et al. 2007 |
| E1-_{CAT} | Maize | Apoplast | Biswas et al. 2006; |
| | | | Ransom et al. 2007 |
| E1 | Potato | Apoplast | Dai et al. 2000 |
| E1 | Potato | Chloroplast | Dai et al. 2000 |
| E1 | Potato | Vacuole | Dai et al. 2000 |
| E1 | Tobacco | Apoplast | Ziegelhoffer et al. |
| | | | 2001; Dai et al. 2005 |
| E1 | Tobacco | Cytosol | Ziegelhoffer et al. |
| | | | 2001; Dai et al. 2005 |
| E1 | Tobacco | Chloroplast | Dai et al. 2000; |
| | | | Ziegelhoffer et al. |
| | | | 2001; Dai et al. 2005 |
| E1 | Tobacco | ER | Dai et al. 2005 |
| E1-cd | Tobacco | Apoplast | Ziegelhoffer et al. |
| | | | 2001 |
| E1-cd | Tobacco | Cytosol | Ziegelhoffer et al. |
| | | | 2001 |
| E1-cd | Tobacco | Chloroplast | Ziegelhoffer et al. |
| | | | 2001 |
| E2 | Tobacco | Cytosol | Ziegelhoffer et al. |
| | | | 1999 |
| E2 | Potato | Cytosol | Ziegelhoffer et al. |
| | | | 1999 |
| E2 | Alfalfa | Cytosol | Ziegelhoffer et al. |
| | | | 1999 |
| E3 | Tobacco | Cytosol | Ziegelhoffer et al. |
| | | | 1999 |
| E3 | Potato | Cytosol | Ziegelhoffer et al. |
| | | | 1999 |
| E3 | Alfalfa | Cytosol | Ziegelhoffer et al. |
| | | | 1999 |
| CBHI | Tobacco | Cytosol | Dai et al. 1999 |
| Human acid BG | Tobacco | Cytosol | Reggi et al., 2005 |
| Maize BG | Tobacco | Chloroplast | Kiran et al. 2006 |
| B. fibrisolvens | Tobacco | Vacuole | Yao 2004 |
| H17c BG | | | |
| B. fibrisolvens | Maize | Vacuole | Ransom 2007 |
| H17c BG | | | |
| xynA | Barley | Cytosol | Patel et al. 2000 |
| xynZ | Tobacco | Apoplast | Herbers et al. 1995 |
| XynA-cd | Tobacco | Cytosol | Kimura et al. 2003a |
| XynA-cd | Rice | Cytosol | Kimura et al. 2003b |
| xynB | Potato | Cytosol | Yang et al. 2007 |
| xynB | Potato | Apoplast | Yang et al. 2007 |

The apoplast is a popular and excellent compartment for targeting because it is spacious and can thus accumulate large quantities of foreign proteins (Ziegler et al. 2000); for E1 endoglucanase and possibly other cellulases, its pH is also similar to its native source, A. cellulolyticus, 5.5-5.6. However, for the reasons outlined above, the ER may be an even better localization goal.

The plant could have a suite of hydrolysis enzymes selected based on optimal synergy, each targeted to a different compartment (apoplast, chloroplast, ER, mitochondria, vacuole, etc.). Alternatively, if the same enzyme were targeted to several compartments in the same plant, production could be maximized (Ragauskas et al. 2006). Recently, this approach was investigated with xylanase targeted to both chloroplasts and peroxisome (Hyunjong et al. 2006).

### 6. Other Approaches

To be economically viable as a technology, plant-produced hydrolysis enzymes must be less expensive than those produced in microbes while retaining the same activity. It would be ideal if the plants used to produce the enzymes had less lignin or were more amenable to pretreatment, requiring less pretreatment and/or chemicals.

### 6.1 Microbial Engineering

Researchers have been steadily increasing both the efficiency of production and the activity of the enzymes using synthetic enzymes and engineered microbes, resulting in a dramatic decrease in the cost of cellulase production in microbes (Knauf and Moniruzzaman 2004; Ragauskas et al. 2006). Microbial molecular geneticists also have a goal to produce designer microbes that secrete all the necessary hydrolysis enzymes and are able to use all the resulting sugars as a feedstock for fermentation in an effort to achieve “consolidated bioprocessing” (Lynd et al. 2005). This technology features cellulose production, hydrolysis and fermentation in one step. Ideally, the enzymes produced by these organisms would be modified to have optimized activity both individually and in specific combinations for maximum synergy. Although potentially feasible, this technology has not been realized to date.

### 6.2 Lignin Pathway Manipulation

As pretreatment is still a costly necessity of the cellulosic ethanol process, reducing or eliminating this need is a key goal. One way to achieve it could be reduction of lignin or modification of its structure in the feedstock biomass (Ragauskas et al. 2006). Lignin contains few components, so regulation of its pathway genes should be relatively straightforward. It is derived from three precursors, para-courmaryl, coniferyl and sinapyl alcohols, which are synthesized in separate but interconnected pathways that are also involved in other cellular functions, including defense (Ragauskas et al. 2006). Other industries are interested in modifying plant lignin for additional purposes, such as increasing digestibility, decreasing bleaching necessity and reducing chemical usage (Boudet 2000; Dean 2004; Ralph et al. 2006; Ralph 2006).

Down-regulation of a major lignin pathway gene, 4-coumarate 3-hydroxylase (C3H), in alfalfa (Medicago sativa) resulted in a dramatic shift in the lignin profile and consequent altered lignin structure that were speculated to explain an earlier study (Reddy et al. 2005) reporting improved digestibility of C3H-deficient alfalfa lines in ruminants (Ralph et al. 2006). Down-regulation of cinnamyl alcohol dehydrogenase (CAD) in alfalfa also resulted in increased in situ digestibility and modified lignin composition although no overall reduction in lignin content (Baucher et al. 1999). Suppression of O-methyl transferase (OMT) in tobacco (Nicotiana tabacum) resulted in increased biomass production and a shift from the structural to non-structural fraction in biomass partitioning, but again no overall decrease in lignin content (Blaschke et al. 2004). Tobacco with reduced CCR showed a decrease in lignin and also an increase in xylose and glucose associated with the wall (Chabannes et al. 2001).

Experiments in Populus spp. have studied alteration in lignin composition as well. When cinnamoyl CoA reductase (CCR) was down-regulated in poplar, Clostridium cellulolyticum was better able to digest the polysaccharides, and twice as much fermentable sugar was released (Boudet et al. 2003). Down-regulation of 4-courmarate CoA ligase (4CL) in P. tremuloides resulted in a 45% decrease in lignin and a corresponding 15% increase in cellulose (Li et al. 2003; Hu et al. 1999) which was enhanced even further (52% less lignin and 30% more cellulose) when coniferaldehyde 5-hydroxylase (CAld5H) was also present (Li et al. 2003). Down-regulation of CAD resulted in improved lignin solubility in alkaline medium, allowing easier delignification; this could decrease costs associated with pretreatment because fewer chemicals are needed (Pilate et al. 2002).

While these results are encouraging, it is important to remember that lignin is essential for structure and for defense against pathogens and insects. Alteration of lignin structure and amount must be done without sacrificing vital needs of the plants involved.

### 6.3 Up-Regulation of Cellulose Pathway Genes to Increase Sugar Content

Several research groups are actively involved in elucidating cellulose synthesis in plants (Kawagoe and Delmer 1997; Arioli et al. 1998; Bolwell 2000; Persson et al. 2005; Andersson-Gunneras et al. 2006; Haigler 2006). So far, most modifications in the pathway genes have been used in basic research in the study of cellulose synthesis. However, manipulation of pathway component genes to increase polysaccharide content for the purposes of improved feedstock production holds promise and should be an area of greater research activity.

### 6.4 Delayed Flowering to Increase Biomass

Increasing feedstock biomass is one way to increase the amount of cellulose available for hydrolysis and fermentation. A promising strategy is to increase the duration of the vegetative state, which can be achieved by engineering plants that have delayed flowering. A floral repressor gene identified in Arabidopsis, FLOWERING LOCUS C (FLC), maintains a vegetative state unless the plant is exposed to vernalization; in this event, the gene is turned off and the plant flowers (Sheldon et al. 1999; Michaels and Amasino 1999). This gene was used to engineer tobacco, with a successful late-flowering result, and a concomitant increase in biomass (Salehi et al. 2005). Accordingly, work is in progress to test expression, delay in flowering and increase in biomass in the biomass crop maize (Sticklen laboratory, unpublished).

### 6.5 Genetic Manipulation to Increase Biomass

Other ways to increase plant biomass could include modification of plant growth regulators such as gibberellins. Hybrid poplar displayed improved growth and biomass when gibberellin biosynthesis was increased (Eriksson et al. 2000). Another strategy could be to adjust the plant's physiology to harness or enhance biological functions such as rate of photosynthesis (Richards 2000); uptake of CO₂, nitrogen and other resources; utilization of nutrients, oxygen and water; respiration; synchronization of circadian clock and external light-dark cycle (Dodd et al. 2005); and carbon allocation (Luo et al. 1997). These processes could be augmented through genetic manipulation of the plants, which has the potential to boost growth and thus biomass production. In a study on transformation of rice to increase endosperm activity of ADP-glucose pyrophosphorylase (AGP), a key enzyme in starch biosynthesis, an unexpected 20% increase in plant biomass was observed (Smidansky et al. 2003).

### 7. Conclusion

The U.S. needs a competitive substitute for fossil fuels; ethanol biofuel is an attractive choice in a suite of alternatives. It is more environmentally benign and because its use can decrease dependency on foreign oil. It can be produced from starch or enzymatic hydrolysis of cellulosic biomass. Production of ethanol involves fermentation of sugars, which can come from plants as simple sugars, starches, or complex structural polysaccharides of the plant cell wall.

Several microorganisms and the enzymes they produce have been studied for their ability to degrade cell walls. The enzymes necessary for this process include cellulases (endo- and exo-glucanases and β-glucosidases) and hemicellulases (most importantly xylanases). Ligninases are also produced by some organisms but are less well understood.

Biomass ethanol production involves several steps: production of hydrolysis enzymes; biomass pretreatment; enzymatic hydrolysis; fermentation of the resulting sugars and distillation. Although biomass is abundant, it has not been traditionally used because of the costs involved in pretreatment and enzyme production. There are few pilot facilities at this stage, but more are planned. Roadblocks stand in the way of this technology becoming mature and economically feasible, including the high costs of enzymes and pretreatment.

Pretreatment is necessary because the cellulose is locked away inside microfibrils, which are wrapped in hemicellulose and lignin, making it inaccessible to hydrolysis enzymes. Current pretreatments include dilute acid, flow-through, ammonia fiber explosion (AFEX), ammonia recycle percolation, steam water explosion, lime, and organosolv pulping.

Cellulases break down cell walls in a three-step process: endoglucanase randomly cleaves crystalline regions; exoglucanase (also called cellobiohydrolase) breaks these chains down further into cellobiose (two glucose molecules) units; and β-glucosidase completes the reaction by releasing the single glucose units from cellobiose.

Cellulase enzymes are produced in microbial fermentation tanks, but could be produced in crops to reduce costs. An ideal candidate is the biomass crop maize: it produces a large amount of biomass, is already widely grown and used as a biofactory for enzymes and other industrial products, and systems for production and distribution are already in place. In addition, enzymes could be targeted to different subcellular compartments to produce a suite of cellulase enzymes in the same plant, optimize expression or avoid hydrolysis until required. In addition to using crops as enzyme biofactories, other solutions to the problems mentioned above include reducing or altering the plants' lignin content to make it more amenable to pretreatment, and microbial engineering. Genetic approaches for increasing biomass include up-regulation of cellulose pathway enzymes, delay in flowering, and manipulation of plant growth regulators. Several of these research avenues will need to be combined before the technology can be perfected. However, the major landmarks reached to date indicate that the transition from fossil fuels to biofuels is achievable.

Production of the hydrolysis enzymes E1 and BG has been achieved in several plants: The catalytic domain of the thermostable E1 endo-1,4-β glucanase of A. cellulolyticus (Tucker et al. 1989; Baker et al. 1994) has been successfully produced in Arabidopsis (Ziegler et al. 2000), tobacco (Ziegelhoffer et al. 2001), rice (Oraby et al. 2007), and maize (Biswas et al. 2006; Ransom et al. 2007) and the full-length peptide in potato (Dai et al. 2000b; Dai et al. 2005) and tobacco (Ziegelhoffer et al. 2001). Human, maize and B. fibrisolvens H17c β-glucosidases have been successfully expressed in tobacco (Reggi et al. 2005; Kiran et al. 2006; Yao 2004) and the B. fibrisolvens H17c β-glucosidase in maize (See Chapter IV. BG RESULTS).

### OBJECTS

The objects of the present invention are to:
1) Produce the cellulases endoglucanase E1 from Acidothermus cellulolyticus and β-glucosidase from Butyrivibrio fibrisolvens H17c in transgenic maize;
2) Perform molecular analyses to show that these enzymes were produced in the plants;
3) Test the enzymes' activities and test their ability to convert cellulose and AFEX-pretreated biomass to fermentable sugars;
4) Combine plant-produced cellulases to test ability to convert cellulose and AFEX-pretreated biomass to fermentable sugars; and
5) Confirm the feasibility of producing biologically-active hydrolysis enzymes in the crop plant maize and demonstrate their usefulness in conversion of lignocellulosic biomass to fermentable sugars for reducing costs associated with ethanol fuel.

These and other objects of the present invention will become increasingly apparent with reference to the following drawings and preferred embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. pMZ766-E1_{CAT}. CaMV 35S: Cauliflower Mosaic Virus (CaMV) 35S promoter; Ω: tobacco mosaic virus (TMV) translational enhancer; Pr1aSP: tobacco pathogenesis-related protein (apoplast targeting signal); E1-cat: coding sequence of the catalytic domain of endo-1,4-β-gluccanase E1 from A. cellulolyticus; nos: polyadenylation signal from the nopaline synthase gene.

FIG. 2. pUC1813. CaMV 35S: Cauliflower Mosaic Virus (CaMV) 35 S promoter; ER: endoplasmic-reticulum leading sequence; bglA: gene encoding β-glucosidase; VT: vacuole-targeting sequence; 35S-t: CaMV 35S terminator.

FIG. 3. pBY520 (Xu et al. 1996). Act1-5′ rice actin 5′ region (promoter); HVA1: barley HVA1 gene; pinII: potato proteinase inhibitor II terminator; 35S 5′: Cauliflower Mosaic Virus 35S promoter; bar: bar gene encoding Bialaphos herbicide resistance; nos-3′: nos terminator.

FIG. 4. pDM302. Act1-5′: rice actin (Act1-5′) 5′ region (promoter); bar: bar gene conferring Bialaphos herbicide resistance; nos: nos terminator.

FIG. 5. pGreen. LB: T-DNA left border; 35S: Cauliflower Mosaic Virus (CaMV) 35S promoter; bar: bar gene conferring resistance to Bialaphos; nos: nos terminator; FLC: FLOWERING LOCUS C (FLC) gene; RB: T-DNA right border; nptII: gene conferring bacterial resistance to kanamycin.

FIG. 6. Western blot of 1 μg TSP from transgenic maize plants expressing E1 (T₀). Lanes: +: positive tobacco control; −C: negative maize control (untransformed); 1-9: transgenic maize plants. Invitrogen Magic Mark™ Western Standard used for size markings. Percentages E1 as determined by enzyme activity assay are displayed above bands (Table 2).

FIG. 7. Average conversion of cellulose to glucan using E1 produced from transgenic maize. The substrates used in the experiment were Avicel, carboxymethyl cellulose (CMC) and AFEX-treated corn stover (ACS). The enzymatic hydrolysis was done for a period of 72 h, at 50° C. at 90 rpm. T=TSP from transgenic plants; NT=TSP from non-transgenic control plants. Error bars represent standard deviation from the mean.

FIG. 8. PCR analysis of T₁ maize plants for E1. W: water; −C: non-transgenic maize control DNA; 1-15: T₁ plants from known crosses; +C: plasmid DNA (pMZ766-E1_{CAT}).

FIG. 9. Results of PCR for BG. Individual plants indicated above the lanes are represented by line-plant number. W: reaction containing only water; 1 kb: 1 kb marker (NEB); P: pUC1813.

FIG. 10. Results of PCR of maize transformed with BG for the BG gene. Individual plants indicated above the lanes are represented by line-plant number. W: reaction containing only water; −C: Non-transformed negative control; 1 kb: 1 kb marker (NEB); P: pUC1813.

FIG. 11. Northern blots of maize expressing BG. Individual plants indicated above the lanes are represented by line-plant number. Ethidium-bromide-stained RNA bands corresponding to each of the plants from the gel before are displayed below the blots to show relative loaded amounts. +C: Positive control (plant 10-1); −C: Untransformed maize negative control.

FIG. 12. Southern blots of maize transformed with BG. Genomic DNA (30 μg) and pUC1813 plasmid DNA (1 ng) were digested overnight with [A] BglII (left panel) or NcoI (right panel) or [B-E] NcoI and fractionated on a 1% w/v agarose gel. Individual plants indicated above the lanes are represented by line-plant number. 1 kb: 1 kb molecular weight marker (NEB); P: pUC1813 positive control; −C: Untransformed maize inbred line HIII. Panels D and E represent the same samples but different exposure times (8 h and ½ h, respectively).

FIG. 13. Top panel: Glucose (g/L) released from 1% cellobiose at 0 min, 1 h, 7.25 h and 26.5 h, using 570 μl maize TSP from plants expressing BG. Data have been adjusted by subtracting the substrate blank. Bottom panel: Glucose (g/L) released per mg protein in TSP. Numbers below the bars represent maize plants expressing BG. HII: Untransformed maize negative control.

FIG. 14. Glucose (g/L) released from 1% cellobiose at 0 min, 1 h, 7.25 h and 26.5 h, using 570 μl maize TSP from plants expressing BG. Data have been adjusted by subtracting the substrate blank. Top panel: Lines represent different maize plants expressing BG; numbers below the lines represent sample collection time points; HiII: Untransformed maize negative control. Bottom panel: T: transgenic plants; NT: non-transgenic plants. Dashed lines represent 95% confidence intervals; solid lines represent polynomial regression lines; dots represent individual data points.

FIG. 15. Sugar determination via DNS on a 0.5% CMC plate. SPC: Spezyme CP; MFX: Multifactorial xylanase; BG: Novozyme 188.

FIG. 16. Sugar determination via DNS on a 1% cellobiose plate. Bottom panel: Undiluted Novozyme 188 is not included to show the other dilutions in more detail.

FIG. 17. Difference between glucose released in commercial enzyme (Novozyme 188) vs. substrate background (blank).

FIG. 18. Sugar determination via glucose analyzer on a 1% cellobiose plate after hydrolysis.

FIG. 19. Glucose released from 1% CMC after 1 h hydrolysis using TSP from plants expressing E1 or CBHI.

FIG. 20. Glucose released from 1% CMC or Avicel after 24 h hydrolysis using TSP from plants expressing E1 or CBHI. Numbers above 24 h bars indicate times higher than 0 h data.

FIG. 21. Sugar release from 1% Avicel (AV) or CMC after 24 h by application of various combinations of plant TSP containing hydrolysis enzymes, determined by DNS assay. E: E1 (30 μg); C: CBH1 (120 μg); B0.1: BG (15 μg); B0.5: BG (75 μg); E:C:E1 and CBH1 (1:4 ratio); E:C:B0.1: E1, CBH1 and BG (15 μg) (1:4:0.1 ratio); E:C:B0.5: E1, CBH1 and BG (75 μg) (1:4:0.5 ratio); E:C:B1: E1, CBH1 and BG (150 μg) (1:4:1 ratio); AV: 1% Avicel; CMC: 1% CMC.

FIG. 22. Sugar release from 1% Avicel (AV) or CMC after 24 h by application of various combinations of plant TSP containing hydrolysis enzymes, determined by glucose analyzer. E: E1 (30 μg); C: CBH1 (120 μg); B0.1: BG (15 μg); B0.5: BG (75 μg); E:C: E1 and CBH1 (1:4 ratio); E:C:B0.1: E1, CBH1 and BG (15 μg) (1:4:0.1 ratio); E:C:B0.5: E1, CBH1 and BG (75 μg) (1:4:0.5 ratio); E:C:B1: E1, CBH1 and BG (150 μg) (1:4:1 ratio); AV: 1% Avicel; CMC: 1% CMC.

FIG. 23. Average sugar release after subtracting enzyme blanks determined by DNS assay. E:C: E1 and CBH1 (1:4 ratio); E:C:B0.1: E1, CBH1 and BG (15 μg) (1:4:0.1 ratio); E:C:B0.5: E1, CBH1 and BG (75 μg) (1:4:0.5 ratio); E:C:B1: E1, CBH1 and BG (150 μg) (1:4:1 ratio); AV: 1% Avicel; CMC: 1% CMC. E:C:B0.1 for CMC is likely higher than shown; E:C:B0.5 for AV is likely lower than shown; E:C:B1 for both is likely lower than shown.

FIG. 24. Glucose release after subtracting enzyme blanks determined by glucose analyzer. E:C: E1 and CBH1 (1:4 ratio); E:C:B0.1: E1, CBH1 and BG (15 μg) (1:4:0.1 ratio); E:C:B0.5: E1, CBH1 and BG (75 μg) (1:4:0.5 ratio); E:C:B1: E1, CBH1 and BG (150 μg) (1:4:1 ratio); AV: 1% Avicel; CMC: 1% CMC. E:C:B0.1 for CMC is likely higher than shown; E:C:B0.5 for AV is likely lower than shown; E:C:B1 for both is likely lower than shown.

FIG. 25. Sugar release on 1% Avicel after 18 h; enzyme blanks subtracted. E:C: E1 and CBH1 (1:4 ratio); E:C:B0.1: E1, CBH1 and BG (2.3 μg) (1:4:0.1 ratio); E:C:B0.5: E1, CBH1 and BG (11.4 μg) (1:4:0.5 ratio); E:C:B1: E1, CBH1 and BG (22.8 μg) (1:4:1 ratio); E:C:B2: E1, CBH1 and BG (45.5 μg) (1:4:2 ratio).

FIG. 26. Sugar release on 1% CMC after 18 h, enzyme blanks subtracted. E:C:E1 and CBH1 (1:4 ratio); E:C:B0.1: E1, CBH1 and BG (2.3 μg) (1:4:0.1 ratio); E:C:B0.5: E1, CBH1 and BG (11.4 μg) (1:4:0.5 ratio); E:C:B1: E1, CBH1 and BG (22.8 μg) (1:4:1 ratio); E:C:B2: E1, CBH1 and BG (45.5 μg) (1:4:2 ratio).

FIG. 27. Sugar release on 1% Avicel after 18 h; enzyme blanks subtracted. E:C: E1 and CBH1 (1:4 ratio); E:C:B0.1: E1, CBH1 and BG (2.3 μg) (1:4:0.1 ratio); E:C:B0.5: E1, CBH1 and BG (11.4 μg) (1:4:0.5 ratio); E:C:B1: E1, CBH1 and BG (22.8 μg) (1:4:1 ratio); E:C:B2: E1, CBH1 and BG (45.5 μg) (1:4:2 ratio). Data for series “Plant TSP alone” is same as FIG. 25, shown here for comparison.

FIG. 28. Sugar release on 1% CMC after 18 h; enzyme blanks subtracted. E:C:E1 and CBH1 (1:4 ratio); E:C:B0.1: E1, CBH1 and BG (2.3 μg) (1:4:0.1 ratio); E:C:B0.5: E1, CBH1 and BG (11.4 μg) (1:4:0.5 ratio); E:C:B1: E1, CBH1 and BG (22.8 μg) (1:4:1 ratio); E:C:B2: E1, CBH1 and BG (45.5 μg) (1:4:2 ratio). Data for series “Plant TSP alone” is same as FIG. 26, shown here for comparison.

FIG. 29. Sugar release on 1% ACS after 24 h; blanks subtracted. E:C:E1 and CBH1 (1:4 ratio); E:C:B0.1: E1, CBH1 and BG (2.3 μg) (1:4:0.1 ratio); E:C:B0.5: E1, CBH1 and BG (11.4 μg) (1:4:0.5 ratio); E:C:B1: E1, CBH1 and BG (22.8 μg) (1:4:1 ratio); E:C:B2: E1, CBH1 and BG (45.5 μg) (1:4:2 ratio).

FIG. 30. Sugar release on 1% ACS after 24 h; blanks subtracted. E:C:Novo: E1 and CBH1 (1:4 ratio) plus Novozyme 188; SP:B0.1: Spezyme CP(SPC) and BG (2.3 μg); SP:B0.5: SPC and BG (11.4 μg); SP:B1: SPC and BG (22.8 μg); SP:B2: SPC and BG (45.5 μg).

FIG. 31. PCR for FLC of maize plants co-transformed with either XYL1 (X−) or pMSF15. W: Reaction containing only water; −C: nontransgenic negative control maize DNA; P: pGreen plasmid DNA.

FIG. 32. PCR for FLC on maize plants co-transformed with BG and FLC. W: Reaction containing only water; L: 100 bp ladder (NEB), P: pGreen.

FIG. 33. Southern blots of maize co-transformed with pSMF15 (left side) or XYL (right side) and pGreen. HiII: Non-transgenic negative control; P: pSMF15; F: pGreen.

FIG. 34. Northern blots of maize co-transformed with pSMF15 (left side) or XYL1 (X−; right side) and pGreen. Ethidium-bromide stained RNA bands are shown below the blot to show amount loaded. −C and −C(M): Non-transgenic negative control.

FIG. 35. XYL Construct. CaMV 35S: Cauliflower mosaic virus 35S promoter; Ω: tobacco mosaic virus translational enhancer; Pr1a: signal peptide from tobacco pathogenesis-related protein 1a; XYL1: xylanase gene from C. carbonum; nos: nos terminator.

FIG. 36. Maize plants transformed with XYL1 and FLC.

FIG. 37. PCR for XYL in maize and tobacco plants. Numbers followed by M indicate maize plants; numbers followed by T indicate tobacco plants. W: Reaction containing only water; −C: Untransformed maize plant; P: XYL1 plasmid.

FIG. 38. Southern blot of maize and tobacco plants transformed with XYL1 and FLC. Numbers followed by M indicated maize plants; numbers followed by T indicate tobacco plants. P: XYL1 plasmid; −C: non-transformed maize (M) or tobacco (T).

FIG. 39. Northern blot of maize and tobacco plants transformed with XYL1 and FLC. Numbers followed by M indicated maize plants; numbers followed by T indicate tobacco plants. The probe was a 397 bp PCR-generated fragment of the XYL1 gene. Upper panel: blot probed with PCR-labeled DIG probe; bottom panel: same blot probed with random primed DIG labeled probe. −CM: non-transformed maize; −CT non-transformed tobacco. Ethidium bromide-stained bands from the agarose gel prior to transfer are shown below the blot to show relative amounts of RNA loaded.

FIG. 40. RT-PCR of maize and tobacco plants transformed with XYL1 and FLC. Numbers followed by M indicated maize plants; numbers followed by T indicate tobacco plants. Top panel: RT reaction; bottom panel: No-RT control. −CM: non-transformed maize; −CT non-transformed tobacco; M: 100 bp molecular weight marker (NEB).

FIG. 41. Western blot of maize plants transformed with XYL1 and FLC. Letters/numbers above the lanes represent individual plants; 1M, 2M and 4M correspond with the plants that survived to maturity and are represented in the other figures. −C: Untransformed maize negative control. Size markings are indicated on the right side.

FIG. 42. pSMF15. rbcS: rice Rubisco small subunit promoter; TP: rice rbcs chloroplast signal peptide; Syn-cbh1: synthetic CBH1; nos: nopaline synthase 3′ non-coding region.

FIG. 43. Southern blot of maize plants transformed with Syn-CBH1 and FLC. P: pMSF15 plasmid; —C: non-transformed maize.

FIG. 44. Northern blot of maize plants transformed with Syn-CBH1 and FLC. The probe was a 801 bp PCR-generated fragment of the Syn-CBH1 gene. Left panel: blot probed random primed DIG labeled probe; right panel: same blot probed with PCR-labeled DIG probe. −C: non-transformed maize. Ethidium bromide-stained bands from the agarose gel prior to transfer are shown below the blot to show relative amounts of RNA loaded.

FIG. 45. RT-PCR of maize plants transformed with Syn-CBH1 and FLC. Top panel: RT reaction; bottom panel: No-RT control. −C: non-transformed maize; M: 100 bp molecular weight marker (NEB). Arrow indicates 801 bp.

FIG. 46. pZD408. 35S: Cauliflower Mosaic Virus (CaMV) 35S Promoter; SynCBHI: Synthetic CBHI coding region.

### SUMMARY OF THE INVENTION

The present invention provides a transgenic monocot plant protein which degrades lignocellulose wherein the transgenic monocot plant is ground and extracted to produce the plant protein by a method comprising: providing in the monocot plant DNA or at least 80% homologous to the bglA gene, SEQ ID NO: 14, of Butyrivibrio fibrisolvens encoding a β-glucosidase which is operably linked to a nucleotide sequence encoding a signal peptide that directs the β-glucosidase to a subcellular compartment of the transgenic monocot plant; grinding the monocot plant after the β-glucosidase is accumulated; and extracting the plant protein from the ground monocot plant, optionally mixing the plant protein with another plant protein encoding a cellulase, and optionally mixing with a commercially available enzyme produced by a bacterium. In further embodiments, the DNA encoding the signal peptide is operably linked to an endoplastic-reticulum leading sequence (ER) as the subcellular compartment. In further still embodiments, the promoter is CaMV 35S and the terminator is CaMV 35S-T. In still further embodiments, a second DNA encodes a cellulase is included. In still further embodiments, the Butyrivibrio fibrisolvens is from the rumen of a cow. In further still embodiments, a plant is selected from the group consisting of maize, wheat, barley, rye, hops, hemp, rice and grasses. In further embodiments, the transgenic monocot plant protein is from maize. In still further embodiments, the transgenic monocot plant further includes a DNA encoding a selectable marker operably linked to a constitutive promoter. In further still embodiments, the DNA encoding the selectable marker provides the transgenic monocot plant with resistance to an antibiotic, an herbicide, or to environmental stress. In still further embodiments, the DNA encoding resistance to the herbicide is a DNA encoding phosphinothricin acetyl transferase which confers resistance to the herbicide phosphinothricin. Further still, the subcellular compartment is the apoplast of the plant.

The present invention further provides a method for producing transgenic monocot plant proteins which degrade lignocellulose wherein the transgenic monocot plant is ground and extracted to produce the plant proteins comprising: providing a first transgenic monocot plant which includes the DNA or at least 80% homologous to bglA gene, SEQ ID NO: 1, of Butyrivibrio fibrisolvens encoding a β-glucosidase to a subcellular compartment of the transgenic monocot plant, and a second transgenic monocot plant encoding an enzyme other than the β-glucosidase which degrades cellulase; mating by sexual fertilization the first and the second transgenic monocot plants to produce a third transgenic monocot plant which includes the DNA encoding the β-glucosidase and the DNA encoding the enzyme other than β-glucosidase; grinding the monocot plant after the enzymes have accumulated; and extracting the plant proteins. In further embodiments, the DNA encoding the cellulase and the DNA encoding the β-glucosidase are each operably linked to the signal peptide operatively linked to an endoplastic reticulum leading sequence (ER) as the subcellular compartment. In further still embodiments, the promoter is CaMV 35S and the terminator is CaMV 35S-T. In still further embodiments, the DNA encoding the β-glucosidase is a bglA gene of Butyrivibrio fibrisolvens. In further still embodiments, the plant is selected from the group consisting of maize, wheat, barley, rye, hops, rice and grasses. In still further embodiments, the transgenic monocot plant is maize. Further still, the first, second, or both transgenic monocot plants further include a DNA encoding a selectable marker operably linked to a constitutive promoter. Still further, the DNA encoding the selectable marker provides the transgenic monocot plant with resistance to an antibiotic, an herbicide, or to environmental stress. In further embodiments, the DNA encoding resistance to the herbicide is a DNA encoding phosphinothricin acetyl transferase which confers resistance to the herbicide phosphinothricin. In still further embodiments, the subcellular compartment is apoplast endoplasmic reticulum vacuole, chloroplast or mitochondria of the plant. In further embodiments, transformed progeny of the third transgenic monocot plant which includes the DNA encoding the β-glucosidase and the DNA encoding the enzyme other than β-glucosidase are mated by sexual fertilization to a transgenic monocot plant selected from the group consisting of the first, second, and third transgenic monocot plants to produce a transgenic monocot plant comprising different copies of the DNA encoding the β-glucosidase and different copies of the DNA encoding the cellulase.

The present invention further provides a method for converting lignocellulose in a transgenic monocot plant material to fermentable sugars comprising: providing a transgenic monocot plant which includes the DNA or at least 80% homologous to bglA gene, SEQ ID NO: 14, of Butyrivibrio fibrisolvens encoding a β-glucosidase, which is operably linked to a nucleotide sequence encoding a signal peptide that directs the β-glucosidase to a subcellular compartment of the transgenic monocot plant; growing the transgenic monocot plant for a time sufficient for the transgenic monocot plant to accumulate a sufficient amount of the β-glucosidase in the subcellular compartment of the transgenic monocot plant; harvesting the transgenic monocot plant which has accumulated the β-glucosidase in the subcellular compartment of the transgenic monocot plant; grinding the transgenic monocot plant for a time sufficient to produce the transgenic monocot plant material wherein the β-glucosidase and transgenic monocot plant are released from the subcellular compartment; incubating the transgenic monocot plant material for a time sufficient for the β-glucosidase in the plant material to produce the fermentable sugars from the lignocellulose in the transgenic monocot plant material; and extracting the fermentable sugars from the transgenic monocot plant material. In further embodiments, the DNA encoding the signal peptide is operably linked to an endoplastic reticulum leading sequence (ER) as the subcellular compartment. In further still embodiments, the promoter is CaMV 35S and the terminator is CaMV 35S-T. Further still, a second DNA encoding a cellulase is included. In still further embodiments, the Butyrivibrio fibrisolvens is from the rumen of a cow. In further still embodiments, the plant is selected from the group-consisting of maize, wheat, barley, rye, hops, hemp, rice and grasses. In further embodiments, the transgenic monocot plant further includes a DNA encoding a selectable marker operably linked to a constitutive promoter. In still further embodiments, the DNA encoding the selectable marker provides the transgenic monocot plant with resistance to an antibiotic, an herbicide, or to environmental stress.

In further still embodiments, the DNA encoding resistance to the herbicide is a DNA encoding phosphinothricin acetyl transferase which confers resistance to the herbicide phosphinothricin. Further still, the subcellular compartment is the apoplast of the plant. In still further embodiments, further comprising adding a plant material from a non-transgenic monocot plant. In further still embodiments, the steps of fermenting the sugars to ethanol. In still further embodiments, the monocot plant has DNA encoding the flowering locus C (FLC) gene.

The present invention further provides a transgenic monocot plant protein which degrades lignocellulose wherein the transgenic monocot plant is ground and extracted to produce the plant protein by a method comprising: providing in the monocot plant DNA encoding a xylanase having a DNA sequence of at least 80% homologous to XYL1 gene, SEQ ID NO: 16, which is operably linked to a nucleotide sequence encoding a signal peptide that directs the xylanase to a subcellular compartment of the transgenic monocot plant; grinding the monocot plant material after the xylanase has accumulated; and extracting the plant protein with another plant protein encoding a cellulase and optionally mixing with a commercially available enzyme produced by a bacterium. In further embodiments, the DNA encoding the signal peptide is operably linked to an endoplastic-reticulum leading sequence (ER) as the subcellular compartment. In still further embodiments, the promoter is CaMV 35S or Rubisco rbcS, and the terminator is CaMV 35S-T. In further still embodiments, DNA encoding the xylanase is the XYL1 gene. In further embodiments, the plant is selected from the group consisting of maize, wheat, barley, rye, hops, hemp, rice and grasses. In still further embodiments, the plant is maize. In further still embodiments, the transgenic monocot plant further includes a DNA encoding a selectable marker operably linked to a constitutive promoter. Further still, the DNA encoding the selectable marker provides the transgenic monocot plant with resistance to an antibiotic, an herbicide, or to environmental stress. Still further, the DNA encoding resistance to the herbicide is a DNA encoding phosphinothricin acetyl transferase which confers resistance to the herbicide phosphinothricin. In further embodiments, the subcellular compartment is the apoplast of the plant.

The present invention still further provides a method for producing a transgenic monocot plant proteins which degrade lignocellulose wherein the transgenic monocot plant is ground and extracted to produce the plant proteins material comprising: providing a first transgenic monocot plant which includes the DNA encoding a xylanase having a DNA sequence at least 80% homologous to XYL1 gene, SEQ ID NO: 16, to a subcellular compartment of the transgenic monocot plant, and a second transgenic monocot plant encoding an enzyme other than the xylanase which degrades cellulose; mating by sexual fertilization the first and the second transgenic monocot plants to produce a third transgenic monocot plant which includes the DNA encoding the xylanase and the DNA encoding the enzyme other than xylanase; grinding the monocot plant after the enzymes have accumulated; and extracting the plant proteins. In still further embodiments, the DNA encoding the cellulase and the DNA encoding the xylanase are each operably linked to the signal peptide operatively linked to an endoplastic reticulum leading sequence (ER) as the subcellular compartment. In further still embodiments, the promoter is CaMV 35S and the terminator is CaMV 35S-T. In further still embodiments, the DNA encoding the xylanase is the XYL1 gene. In still further embodiments, the plant is selected from the group consisting of maize, wheat, barley, rye, hops, rice and grasses. In further embodiments, the transgenic monocot plant is maize. Further still, the first, second, or both transgenic monocot plants further include a DNA encoding a selectable marker operably linked to a constitutive promoter. In still further embodiments, the DNA encoding the selectable marker provides the transgenic monocot plant with resistance to an antibiotic, an herbicide, or to environmental stress. In further still embodiments, the DNA encoding resistance to the herbicide is a DNA encoding phosphinothricin acetyl transferase which confers resistance to the herbicide phosphinothricin. In still further embodiments, the subcellular compartment is apoplast endoplasmic reticulum vacuole, chloroplast or mitochondria of the plant. In further embodiments, transformed progeny of the third transgenic monocot plant which includes the DNA encoding the xylanase and the DNA encoding the enzyme other than xylanase are mated by sexual fertilization to a transgenic monocot plant selected from the group consisting of the first, second, and third transgenic monocot plants to produce a transgenic monocot plant comprising multiple copies of the DNA encoding the xylanase and multiple copies of the DNA encoding the enzyme other than the xylanase.

The present invention still further provides a method for converting lignocellulose in a transgenic monocot plant material to fermentable sugars comprising: providing a transgenic monocot plant which includes the DNA encoding a xylanase having a DNA sequence at least 80% homologous to XYL1 gene, SEQ ID NO: 16, which is operably linked to a nucleotide sequence encoding a signal peptide that directs the xylanase to a subcellular compartment of the transgenic monocot plant; growing the transgenic monocot plant for a time sufficient for the transgenic monocot plant to accumulate a sufficient amount of the xylanase in the subcellular compartment of the transgenic monocot plant; harvesting the transgenic monocot plant which has accumulated the xylanase in the subcellular compartment of the transgenic monocot plant; grinding the transgenic monocot plant to produce the transgenic monocot plant material, wherein the xylanase and transgenic monocot plant are released from the subcellular compartment; incubating the transgenic monocot plant material for a time sufficient for the xylanase in the plant material to produce the fermentable sugars from the lignocellulose in the transgenic monocot plant material; and extracting the fermentable sugars from the transgenic monocot plant material. In further embodiments, the DNA encoding the signal peptide is operably linked to an endoplastic reticulum leading sequence (ER) as the subcellular compartment. In further still embodiments, the promoter is CaMV 35S and the terminator is CaMV 35S-T. In still further embodiments, DNA encoding the xylanase is the XYL1 gene. In further still embodiments, the plant material is from the group consisting of maize, wheat, barley, rye, hops, hemp, rice and grasses. In further embodiments, the plant material is maize. In still further embodiments, the transgenic monocot plant further includes a DNA encoding a selectable marker operably linked to a constitutive promoter. In further still embodiments, the DNA encoding the selectable marker provides the transgenic monocot plant with resistance to an antibiotic, an herbicide, or to environmental stress. In still further embodiments, the DNA encoding resistance to the herbicide is a DNA encoding phosphinothricin acetyl transferase which confers resistance to the herbicide phosphinothricin. In further embodiments, the subcellular compartment is the apoplast of the plant. In further embodiments, the steps of fermenting the sugars to ethanol. In still further embodiments, the transgenic monocot plant has DNA encoding the flowering locus C (FLC) gene.

### DESCRIPTION OF THE PREFERRED EMBODIMENTS

All patents, patent applications, government publications, government regulations, and literature references cited in this specification are hereby incorporated herein by reference in their entirety. In case of conflict, the present description, including definitions, will control.

The DNA's in the present application for the enzymes are at least 80, 90 or 100 percent homologous to the DNAs of the SEQ ID NOS: 14 and 16. This is also true of the enzymes encoded, SEQ ID NOS: 15 and 17, so long as they continue to function to degrade cellulase.

The following examples are intended to promote a further understanding of the present invention.

### EXAMPLES

### II. Materials and Methods

### 1. Transformation Vectors

### 1.1 Genes of Interest

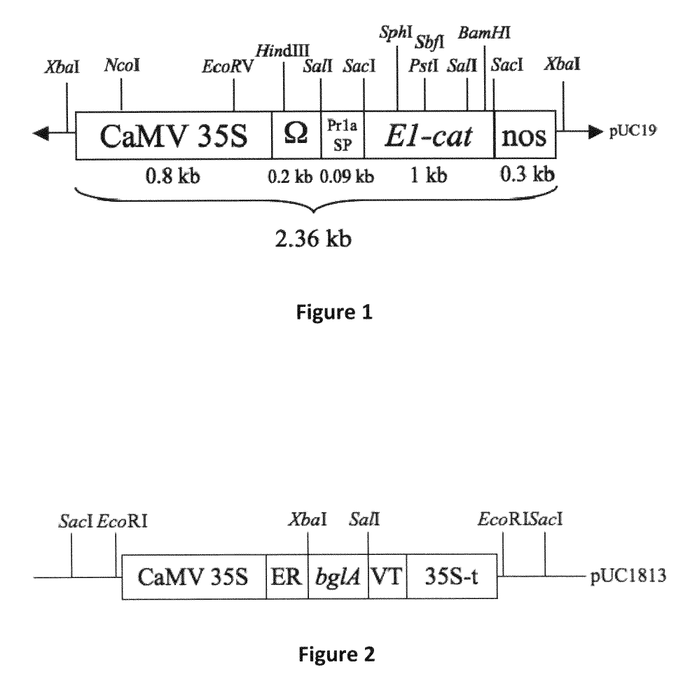

pMZ766-E1_{CAT}. Vector pMZ766-E1_{CAT} (Ziegler et al. 2000) encodes the catalytic domain of endo-1,4-β-glucanase E1 from A. cellulolyticus, targeted to the apoplast with the signal peptide from tobacco pathogenesis-related protein 1a (Pr1a), under regulation of the CaMV 35S promoter, the tobacco mosaic virus translational enhancer (Ω), and the polyadenylation signal from the nopaline synthase gene (3′ nos) (FIG. 1).

pUC1813. Vector pUC1813 (Yao 2004) contains the Cauliflower Mosaic Virus (CaMV) 35S promoter, endoplasmic-reticulum leading sequence (ER); bglA gene encoding Butyrivibrio fibrisolvens H17c β-glucosidase, a vacuole-targeting sequence (VT) and the CaMV 35S terminator (FIG. 2).

### 1.2 Selectable Markers

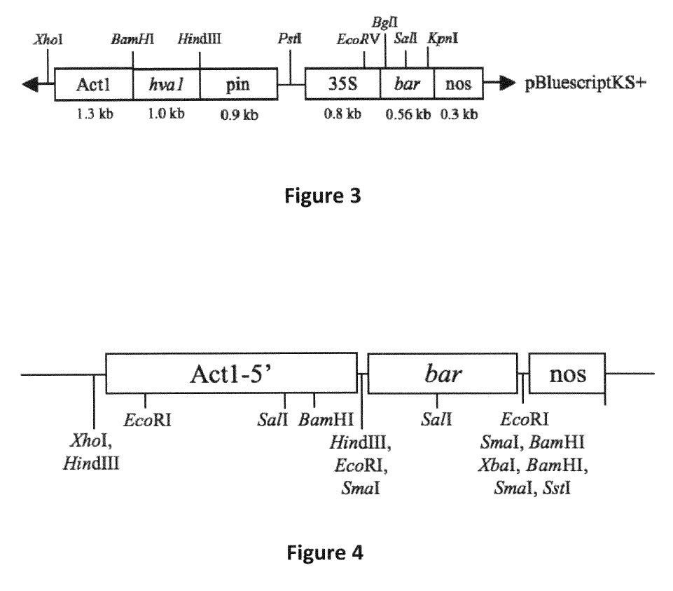

pBY520. Vector pBY520 (Xu et al. 1996) contains the barley HVA1 coding sequence regulated by the rice actin 1 (Act1) promoter and potato proteinase inhibitor II (pinII) terminator, as well as the bar coding sequences regulated by the CaMV35S promoter and nos terminator (FIG. 3).

pDM302. Vector pDM302 (Cao et al. 1992) contains the bar coding sequence under the control of the Act1 promoter and nos terminator (FIG. 4).

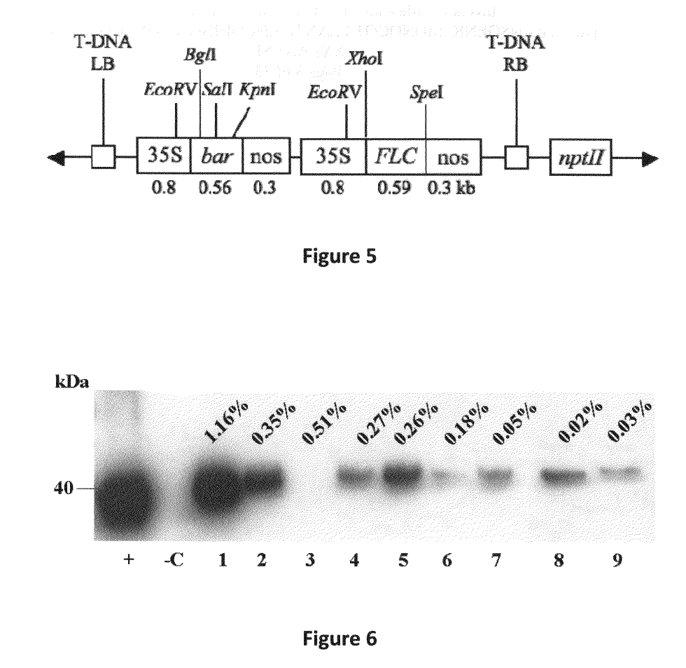

pGreen. Vector pGreen (Hellens et al. 2000) is a binary vector; it contains the bar gene regulated by the 35S promoter and nos terminator and the FLOWERING LOCUS C (FLC) gene regulated by the 35S promoter and nos terminator. In addition it has T-DNA left and right borders and carries the nptII gene for bacterial resistance to kanamycin (FIG. 5).

### 2. Maize Transformation, Acclimation and Care

Highly proliferating, immature-embryo-derived Type II embryogenic callus (Armstrong et al. 1991) was used in transformation experiments. Two to four hours prior to bombardment, callus was transferred to 1.5-cm circles in the center of a Petri dish containing an osmotic (Vain et al. 1993; conditioning) medium. Conditioned callus was bombarded with ethanol-washed tungsten particles combined with a total of 10 μg of 1:1 mixture of two plasmids (one containing the gene of interest, the other containing the selectable marker gene), according to the manufacturer's protocol (BioRad PDS 1000/He® Biolistic gun) at a pressure of 1100 PSI.

The bombarded callus was kept on the same conditioning medium for 24 hours, transferred to callus proliferation medium (Armstrong and Green 1985; Armstrong et al 1995; Chu et al. 1975) for five days, and then placed on selection medium containing 2 mg/L Bialaphos, where they were maintained for six to eight weeks with biweekly subcultures onto fresh medium. All cultures were maintained in the dark. The detected Bialaphos-resistant surviving callus clones were placed in regeneration medium (Biswas et al. 2006) and exposed to continuous light (60 μmol quanta m-²·s-¹ from cool-white 40 W Econ-o-watt fluorescent lamps; Philips Westinghouse, USA) for four to six weeks. Plantlets were transferred further to rooting medium containing 2 mg/L Bialaphos selectable herbicide (Biswas et al. 2006), and maintained for two to four weeks under the above light conditions.

Rooted plantlets 8-10 cm in height were transferred to pots containing soil. Pots were kept covered with plastic bags to maintain humidity, and acclimated in the growth chamber. When plants showed new growth, they were transplanted to 2- or 5-gallon pots and either kept in the growth chamber until maturity or transferred to the greenhouse under similar conditions.

Fertile T₀ plants were self- or cross-pollinated. In some cases, transgenic ears were pollinated with wild type pollen due to lack of sufficient transgenic pollen. Plants were allowed to mature and seeds were harvested after dry-down when the abscission layer had formed, 35-45 days after pollination.

### 3. DNA Analyses

### 3.1 Extraction

Genomic DNA was extracted from leaf tissue with C-TAB as described (Saghai-Maroof et al. 1984).

### 3.2 PCR

E1. The oligonucleotide primers 5′-GCG GGC GGC GGC TAT TG-3′(SEQ ID NO: 1) and 5′-GCC GAC AGG ATC GAA AAT CG-3′ (SEQ ID NO: 2) were designed, synthesized and used to amplify a 1.0 kb fragment spanning the catalytic domain of the endo-1,4-β-endoglucanase gene.

BG. The oligonucleotide primers 5′ GCA TTG ATC TAG AAT GGA GAA ATG GGC AAG AAT 3′ (SEQ ID NO: 3) (left) and 5′ AAT AAT AGT CGA CAG CGG CTT TGA GCT TAG TCG 3′(SEQ ID NO: 4) (right) were used (Yao 2004); they amplify the entire bglA coding sequence, 2.6 kb. The conditions used in PCR were as follows: 30 cycles of 94° C. for 30 sec, 68° C. for 1.5 min, and 72° C. for 2 min.

HVA1. The oligonucleotide primers 5′-TGG CCT CCA ACC AGA ACC-3′ (SEQ ID NO: 5) (forward) and 5′-ACG ACT AAA GGA ACG GAA AT-3′ (SEQ ID NO: 6) (reverse) (Oraby et al. 2005) were used to amplify a 0.7 kb fragment of the HVA1 gene. The PCR conditions were as follows: 94° C. for 3 min; 35 cycles of 94° C. for 45 s, 56° C. for 45 s, and 72° C. for 45 s; and 72° C. for 10 min.

The PCR products were analyzed by electrophoresis in 0.8% agarose gels containing ethidium bromide, and visualized under a UV light.

### 3.3 Southern Blots

General procedure: Genomic DNA was digested with appropriate restriction endonucleases (see specific entries below) and fractionated on a 1% agarose gel. The gel was depurinated, denatured and neutralized, and blotted onto a Hybond-N+ nylon membrane (Amersham-Pharmacia Biotech, Buckinghamshire, UK) according to the manufacturer's instructions.

E1. Five micrograms of genomic DNA and 10 μg plasmid DNA (pMZ766-E1_{CAT}) were digested with HindIII or SacI. Non-radioactive labeling and detection were carried out with a probe representing the E1-CAT coding region.

BG. Thirty micrograms of genomic DNA and 1 ng plasmid DNA (pUC1813) were digested overnight with either BglII or NcoI, which cut the construct once (NcoI cuts at the beginning of the CaMV 35S promoter; BglII cuts at the end of the bglA coding sequence). Radioactive labeling and detection were carried out with a probe representing the bglA coding region, generated by PCR. Copy number was determined by counting the resulting bands.

### 4. RNA Extraction

Total RNA was extracted using TRIZOL® Reagent (Invitrogen Corporation, Carlsbad, Calif. 92008, Cat. # 15596-026) as specified by the manufacturer.

### 5. Northern Blots

Twenty μg RNA were separated on 1.2% (w/v) agarose-formaldehyde denaturing gels (Sambrook et al. 1989) and blotted onto Hybond-N+ nylon membranes (Amersham-Pharmacia Biotech).

### 6. Labeling, Hybridization and Detection for Southern and Northern Blots

For non-radioactive labeling and detection, the PCR DIG Probe Synthesis Kit (Roche Applied Science, Penzberg, Germany, Cat # 11 636 090 910) and/or DIG High Prime Labeling and Detection Starter Kit II (Roche Applied Science, Penzberg, Germany, Cat # 11585614910) was used according to the kit's instructions to generate a probe labeled with digoxigenin-dUTP. Probe hybridization and immunological detection were carried out using the DIG High Prime DNA Labeling and Detection Starter Kit II (Roche Applied Science, Penzberg, Germany, Cat # 11585614910) with the instructions therein.

Radioactive probe labeling was achieved with α-[³²P]-dCTP (GE Healthcare) with the Random Primers DNA Labeling Kit (Invitrogen, Carlsbad, Calif., Cat. # 18187-013) according to the kit's instructions. For hybridization, PerfectHyb™ Plus Hybridization Buffer (Sigma-Aldrich, St. Louis, Mo. 63178; Cat. # H7033) was used at 62° C. according to the instructions. Detection was done according to standard procedures (Sambrook et al. 1989). Blots were exposed to X-ray film and developed in a Kodak RP X-OMAT Processor.

### 7. Extraction of TSP

E1. TSP was extracted from leaf tissues as described (Ziegelhoffer et al. 2001). Briefly, 100 mg fresh leaf tissue was ground in the sodium acetate grinding buffer and precipitated with saturated ammonium sulfate. Extracts were quantified using the Bradford method (Bradford 1976) using a standard curve generated from bovine serum albumin (BSA). For the large-scale TSP extraction (to check the activity on biomass), an automatic solvent extractor (Dionex) was used. To a total of 9 gm pulverized transgenic maize residue, 60 ml grinding buffer was added and used by the machine to extract TSP. The extracted TSP were precipitated by adding an equal volume of saturated ammonium sulfate and allowing to stand overnight at 4° C. The precipitated TSP was collected by centrifugation and concentrated by re-suspending in 5 ml grinding buffer. This TSP concentrate was measured for activity (described below) and used without any further dilution.

BG. TSP was extracted from leaf tissues as described (Carrão-Panizzi and Bordingnon 2000) and quantified as above. Briefly, extraction buffer (0.05M citrate buffer, pH 4.8, 10% glycerol, protease inhibitor cocktail (Sigma-Aldrich, St. Louis, Mo., Cat. # P9599; or Complete, Mini, EDTA-free Protease Inhibitor Cocktail Tablets, Roche Applied Science, Indianapolis, Ind., Cat. # 11836170001) was added to pulverized (via liquid N₂) leaf tissue in approximately a 2:1 ratio to achieve a viscous slurry. Samples were incubated at room temperature for 1 hr, followed by centrifugation at 15,000×g for 5 min; the supernatant was collected and used in subsequent analyses.

### 8. Activity Assays

### 8.1 MUCase Activity Assay for E1

E1 activity was assessed as described (Ziegelhoffer et al. 2001). Briefly, a series of soluble protein dilutions ranging from 10⁻¹ to 10⁻³ were developed, representing concentrations of 0.1-10 ng/μl. In a 96-well plate, 10 μl samples (representing 1-100 ng TSP) was mixed with 100 μl reaction buffer containing 4-methylumbelliferone β-D-cellobioside (MUC). The fluorophore 4-methylumbelliferone (MU), as the product of E1 hydrolization of the substrate MUC, was measured as follows. Plates were covered with adhesive lids and incubated at 65° C. for 30 minutes. The reaction was stopped with the addition of the stop buffer, and the fluorescence was read at 465 nm using SPECTRAmax M2 device (Molecular Devices Inc., Sunnyvale, Calif.) at an excitation wavelength of 360 nm. After subtracting background fluorescence contributed by the control, activity of each sample was calculated using a standard curve representing 4 to 160 pmol MU and compared to the activity of pure E1 reported in Ziegelhoffer et al. (2001).

### 8.2 IUPAC Assay for BG

BG activity was measured as described (Ghose 1987). TSP from plants was added to assay buffer (0.5M citrate buffer, pH 4.8; 0.015M cellobiose) and incubated at 50° C. with gentle agitation on a rotary shaker (90 rpm). Samples were taken at different times depending on the experiment (see BG RESULTS)

8.3 p-Nitrophenol (pNP) Assay for BG

BG activity was determined by measuring the hydrolysis of p-nitro-phenyl-β-D-glucopyranoside (pNPβG), slightly modified as follows from the procedure described by Cai et al. (1999). The incubation mixture was made of 2 mM pNPβG, 50 mM sodium phosphate buffer (pH 6.5) and 30 μl TSP in a total volume of 100 μl. The reaction was carried out at 40° C. for 15 min and terminated by the addition of 300 μl 1.0 M Na₂CO₃. The amount of p-nitrophenol (pNP) released was determined spectrophotometrically by measuring the absorbance of the solution at 415 nm. Standards between 0-100 nmol pNP were also included. One unit of enzyme activity was defined as the amount of TSP that produced 1 nmol of product per min under the conditions of the assay.

### 8.4 DNS Assay for Reducing Sugars

DNS is a calorimetric reagent that detects reducing sugars, and has been used in standard assays (Miller 1959; Decker et al. 2003). A protocol for using DNS to test hydrolysis of commercial enzyme preparations in a microplate was developed in the Dale laboratory. After a hydrolysis step with Avicel, CMC, xylan, or other substrate, 50 μl samples were taken in a new plate, 100 μl DNS was added, the color developed at 100° C. for 30 min, and a reading taken with a 100-μl sample using a UV spectrophotometer at 540 nm. The readings were compared to glucose standards and the glucose released as well as percent conversion calculated. The assay calculations were standardized for a 4% conversion (IUPAC method), so the amount of enzyme had to be diluted in order to achieve this.

### 8.5 Glucose Analyzer

Samples were put in 1.5 ml microfuge tubes and placed in the glucose analyzer turntable; the machine took the readings directly. At least 500 μl sample is needed; if there was not enough sample, it was diluted by half and the numbers adjusted accordingly.

### 9. Western Analysis

### 9.1 General Procedure

For Western blotting, the Invitrogen NuPAGE® Bis-Tris Discontinuous Buffer System with a 10% NuPAGE® Novex Bis-Tris Pre-Cast Gel was used (Invitrogen, Carlsbad, Calif.). One microgram TSP was run on the gel and blotted onto a nitrocellulose membrane (Amersham Hybond™ ECL™; Amersham-Pharmacia Biotech; Buckinghamshire, UK) according to the manufacturer's instructions. The membrane was blocked with 1×PBS, 5% non-fat dry milk, 0.1% Tween-20 and incubated with primary antibody and secondary enzyme conjugate. The Pierce SuperSignal® West Pico Chemiluminescent Substrate was used for detection following the manufacturer's protocol (Pierce Biotechnology, Rockford, Ill.). The blot was exposed to X-ray film for one minute and developed in a Kodak RP X-OMAT Processor.

### 9.2 Specific Conditions

E1. The primary antibody was monoclonal mouse anti-E1, 1 μg/ml, courtesy of National Renewable Energy Laboratory (NERL). The secondary enzyme conjugate was anti-mouse IgG:HRPO (BD Transduction Laboratories™, BD Biosciences, San Jose, Calif.; 1:2000).

### 10. Pretreatment of Biomass

Milled corn stover (about 1 cm in length) was pretreated using the AFEX technology (Teymouri et al. 2004). In more detail, the crop biomass was transferred to a high-pressure reactor (PARR Instrument Col, IL) with 60% moisture (kg water/kg dry biomass) and liquid ammonia ratio 1.0 (kg of ammonia/kg of dry biomass) was added. The temperature was slowly raised and the pressure in the vessel increased. The temperature was maintained at 90° C. for five minutes before explosively releasing the pressure. The instantaneous drop of pressure in the vessel caused the ammonia to vaporize, causing an explosive decompression and considerable fiber disruption. The pretreated material was kept under a hood to remove residual ammonia and stored in a freezer until further use.

### 11. Conversion Analyses

### 11.1 E1

E1 biomass conversion ability was assessed by measuring the reaction of TSP extracted from E1-expressing corn leaves with amorphous cellulose (CMC), crystalline cellulose (Avicel) and material containing both amorphous and crystalline cellulose, i.e. AFEX-pretreated corn stover (Teymouri et al. 2004).

The enzyme hydrolysis was performed in a sealed scintillation vial. A reaction medium, composed of 7.5 ml of 0.1M, pH 4.8 sodium citrate buffer, was added to each vial. In addition, 60 μl (600 μg) tetracycline and 45 μl (450 μg) cycloheximide were added to prevent the growth of microorganisms during the hydrolysis reaction. The substrate was hydrolyzed at a glucan loading of 1% (w:v). The TSP from the plant producing the E1 was concentrated to 1.8% and 250 μl were added to the substrate. The reaction was supplemented with 64 pNPGU/g glucan (Novozyme 188 Cellobiase from Aspergillus niger, Sigma-Aldrich, St. Loius, Mo., Cat. #C6105) to convert the cellobiose to glucose. Distilled water was then added to bring the total volume in each vial to 15 ml. All reactions were performed in duplicate to test reproducibility. The hydrolysis reaction was carried out at 50° C. with a shaker speed of 90 rpm. About 1 ml of sample was collected at 72 hr of hydrolysis, filtered using a 0.2 mm syringe filter and kept frozen. The amount of glucose produced in the enzyme blank and substrate blank were subtracted from the respective hydrolyzed glucose levels. Hydrolyzate was quantified using Waters HPLC by running the sample in Aminex HPX-87P (Biorad) column, against sugar standards.

### 11.2 Microplate Hydrolysis

Hydrolysis reactions were performed in 96-well microplates. Each well contained a steel bead, 418 μl substrate (0.5 or 1% CMC, Avicel or AFEX-treated corn stover (ACS)), 25 μl 1 M citrate buffer and 38 μl enzyme dilution (or plant TSP) plus water for a total volume of 500 μl. Plates were covered with foil tape and incubated at 50° C., 350 RPM for the time specified.

### 12. Progeny Analyses

T₁ seeds were germinated in vitro on 2 mg/L Bialaphos selection medium (Biswas et al. 2006) to determine segregation ratios of the offspring. Then, PCR analyses using the same primers and conditions as in the T₀ generation were conducted to examine the presence of transgenes in the progeny.

### III. Results for E1

One of the challenges to the use of lignocellulosic biomass as a feedstock for ethanol fuel production is the prohibitive cost of the enzymes needed for its saccharification. One proposed solution has been to produce them in plants instead of microbes. To this end, cellulases from various organisms have been produced in several plants. Specifically, the thermostable E1 (endoglucanase) transgene from Acidothermus cellulolyticus (Baker et al. 1994; Tucker et al. 1989) has successfully been expressed in several plants, including Arabidopsis (Ziegler et al. 2000), potato (Dai et al. 2000b), and tobacco (Ziegler et al. 2000; Dai et al. 2000a); however, none of these are sizeable enough plants to enable large-scale commercial production of enzymes. Maize was therefore chosen in an attempt to remedy this problem.

### 1. Transformation

Transformation and regeneration of plants with pMZ766-E1_{CAT} (E1) and either pBY520 or pDM302 were performed by another researcher (Biswas et al. 2006), who obtained a total of 9 lines, each with 4-15 plants that survived to maturity.

### 2. Molecular and Enzymatic Analyses

In preliminary work (Biswas et al. 2006), integration of the E1 coding sequence was confirmed via PCR, which showed that 31 plants carried the E1 transgene. Southern blotting further verified the integration of the E1 transgene in these plants (Biswas et al. 2006). Northern blots did not show any hybridization with the probe.

Forty plants of seven lines were tested for activity. Among the 31 PCR positive plants, 16 showed biological activity compared to control untransformed plants, as evidenced by percent E1 in plant leaf extract TSP (Table 2). Percentages of E1 in TSP ranged from 0.01% to 1.16%. The assay was able to detect enzyme activity levels as low as 0.01% E1. Western blotting confirmed the translation of E1, also showing differences in the production levels (FIG. 6). In general, the signal strength observed in the Western blot corresponded with the percentage E1 observed in activity assays (FIG. 6), although one plant, 7-6, does not show a band. The nine plants (from four different lines) that showed the highest levels of activity were chosen for further study.

TABLE 2
Mean enzymatic activity and percentage E1 of total plant soluble
proteins produced by transgenic maize plants (T₀).

| | Plant (line-plant number) | | | | | | | | |
|---|---|---|---|---|---|---|---|---|---|
| | 2-8 | 2-5 | 7-6 | 1-11 | 1-1 | 2-3 | 1-13 | 1-12 | 1-10 |
| % E1 | 1.16 | 0.35 | 0.51 | 0.27 | 0.26 | 0.18 | 0.05 | 0.02 | 0.03 |
| N | 5 | 9 | 7 | 6 | 1 | 1 | 3 | 2 | 1 |
| S.D. | 0.147 | 0.308 | 0.349 | 0.156 | N/A | N/A | 0.042 | 0.006 | N/A |
| Activity | 0.464 | 0.1408 | 0.202 | 0.109 | 0.104 | 0.072 | 0.02 | 0.008 | 0.012 |
| (nmol/μg/min) | | | | | | | | | |

N = number of replicates;
S.D. = standard deviation.

### 3. Conversion Analyses

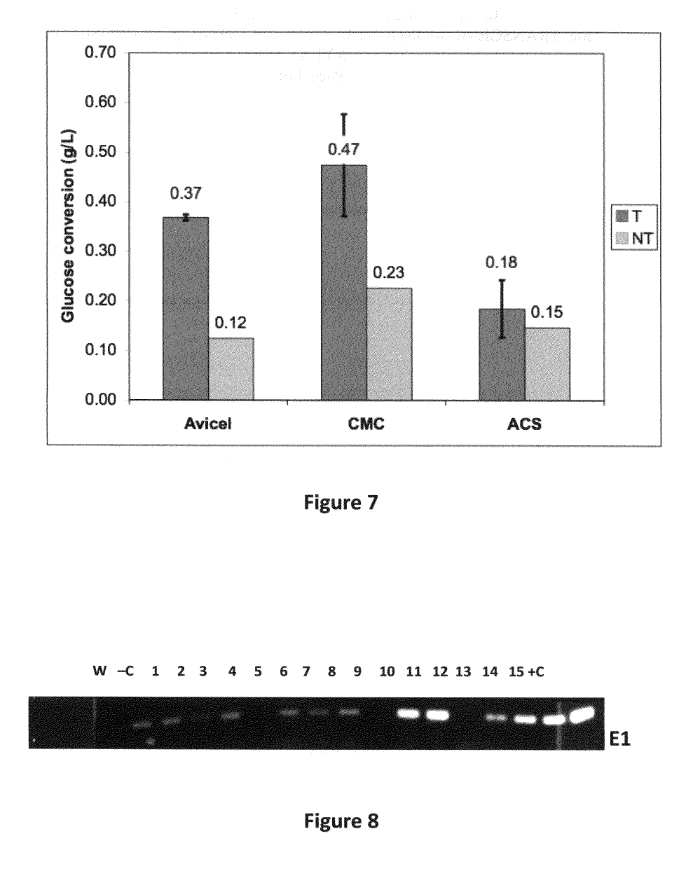

The hydrolytic conversion of cellulose using the plant-produced E1 was confirmed by adding transgenic corn TSP to three types of substrates: CMC, Avicel and AFEX-pretreated corn stover. The conversion of cellulose to glucose ranged from 0.18 to 0.47 g/L when transgenic plant TSP concentrate was used on these substrates (FIG. 7). The highest sugar release, with a mean of 0.47 g/L (after 72 hrs), was observed when the transgenic plant TSP was added to CMC (FIG. 7).

### 4. Second Generation

To obtain second-generation (T₁) transgenic seeds, the plants were self- and/or cross-pollinated in the greenhouse. The crosses that produced the most seeds included 2-5×1-10, 1-12×1-10, 1-7×1-13, 1-10×1-13, 1-13× negative control. None of the self-pollinations resulted in seed. These crosses resulted in 15 progeny that survived to maturity. In addition, another researcher made two successful but not well-documented crosses; one with an unknown plant of line 2, the other with plant 7-6. It is unknown if these were the females or the males, and which were the other parents, or if they were selfed. These crosses resulted in 22 progeny (plants).

PCR analysis confirmed the transmission of the E1 gene to the progeny (FIG. 8). None of the progeny retained the HVA1 gene.

Northern and Western blots of the 22 plants of dubious ancestry showed no hybridization; they were not performed on the other 15 plants. Activity assays were performed on the 15 progeny of known crosses (Table 3).

TABLE 3
Percentage of E1 in second generation (T₁) plants
as determined by activity assay.

| 1^{st} Gen. ♀ | | 1^{st} Gen. ♂ | | 2^{nd} Gen. | |
|---|---|---|---|---|---|
| (line- | % E1 | (line- | % E1 | Plant | |
| plant) | (♀) | plant) | (♂) | (n = 3) | % E1 |
| 2-5 | 0.35 | 1-10 | 0.03 | 1 | 0.0006 |
| | | | | 9* | 0.0040 |
| | | | | 14 | 0.0131 |
| 1-12 | 0.02 | 1-10 | 0.03 | 8* | — |
| 1-7 | — | 1-13 | 0.05 | 4 | — |
| | | | | 7 | — |
| | | | | 13 | 0.0389 |
| | | | | 2 | 0.0014 |
| 1-10 | 0.03 | 1-13 | 0.05 | 3 | — |
| | | | | 11 | — |
| | | | | 15 | — |
| 1-13 | 0.05 | HNPC | N/A | 10 | — |
| | | | | 12* | — |
| | | | | 5* | 0.0483 |

The first four columns show the parents and % E1, while the last two columns show the same information for the second generation.
1-15: Second generation E1 plants;
—: Activity showed less than negative (nontransgenic) control;
HNPC: Nontransgenic variety ‘Honey 'n Pearl’ negative control;
N/A: Not applicable;
*PCR negative for pMZ766-E1_{CAT}

### IV. Results for BG

The microfibrils in plant cell walls are composed of long chains of cellulose; most of this is crystalline but there are some amorphous regions as well. An endoglucanase is needed to randomly cleave the crystalline cellulose to expose the chain ends that the exoglucanase can work on. Once the exoglucanase has reduced the cellulose to cellobiose, β-glucosidase (BG) can catalyze the final step for glucose release. BG is a class of enzyme that breaks β 1→4 linkages between glucose molecules. Because of its vital role in completing the hydrolysis reaction, it is the subject of this set of experiments. The first goal was to transform maize with a gene encoding BG, recover enzymatically active protein from the plants, and show that it is able to convert cellobiose to glucose.

A secondary goal was to try to increase the biomass for greater protein production while simultaneously improving genetic confinement. To this end, the gene FLOWERING LOCUS C (FLC) from Arabidopsis was co-transformed with BG. The FLC gene delays flowering and prolongs the vegetative state; it has been shown to delay flowering and increase biomass production in tobacco (Salehi et al. 2005). In addition, the FLC plasmid, pGreen, contains the linked bar gene for Bialaphos (herbicide) resistance and thus in addition provides a selectable marker. The focus of this work is mainly on BG. Supplementary data on FLC experiments are located in Appendix A.

1. Maize Plants are Transformed with BG and FLC

A total of 150 plates of immature embryo-derived maize callus were bombarded with pUC1813 (containing BG); 140 of these were co-transformed with pGreen (containing bar and FLC) and the remaining 10 were co-transformed with pDM302 (containing bar only). Sixty-three clones resistant to Bialaphos survived the selection medium. Of these, 34 lines were regenerated into 196 plants (Table 4).

TABLE 4
Transformation events and regeneration of T₀
maize plants co-transformed with a combination of
pUC1813 (containing BG) and pDM302 (event 11 only;
containing bar) or pGreen (all other events; containing
FLC and bar).

| | | | | | N |
|---|---|---|---|---|---|
| | | | | | plants |
| | Plates | Resistant | N lines | Line | per |
| Event | bombarded | clones | regenerated | designations | line* |
| 1 | 10 | 0 | — | — | — |
| 2 | 10 | 4 | 2 | 1-2 | 2, 14 |
| 3 | 9 | 0 | — | — | — |
| 4 | 6 | 0 | — | — | — |
| 5 | 5 | 0 | — | — | — |
| 6 | 5 | 7 | 4 | 3-6 | 1, 1, |
| | | | | | 2, 3 |
| 7 | 5 | 3 | 2 | 7-8 | 9, 9 |
| 8 | 10 | 0 | — | — | — |
| 9 | 18 | 24 | 17 | 9-25 | 24, |
| | | | | | 35, |
| | | | | | 16, 7, |
| | | | | | 5, 5, |
| | | | | | 4, 1, |
| | | | | | 2, 6, |
| | | | | | 5, 3, |
| | | | | | 1, 6, |
| | | | | | 4, 6, 1 |
| 10 | 18 | 8 | 5 | 26-30 | 1, 5, |
| | | | | | 5, 3, 1 |
| 11 | 10 | 0 | — | — | — |
| 12 | 17 | 3 | — | — | — |
| 13 | 17 | 14 | 4 | 31-34 | 1, 1, |
| | | | | | 1, 5 |
| Total | 150 | 63 | 34 | | 196 |

*The number of plants per line refers to the line designations, e.g., from lines designated 3-6, there is one plant from line 3, one plant from line 4, two plants from line 5 and three plants from line 6.

### 2. Maize Plants are Transgenic and Express BG

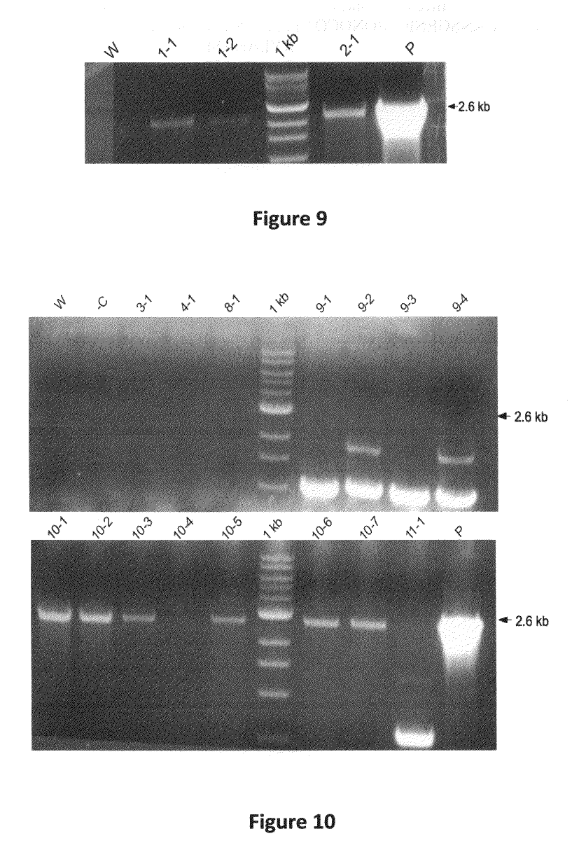

PCR for BG (FIGS. 9 and 10) was performed on plants from eight lines (1, 2, 3, 4, 8, 9, 10, and 11). Bands were detected in five of these lines (1, 2, FIG. 9; 9, 10, 11, FIG. 10) but only three had bands of the correct size, 2.6 kb (1, 2, 10). This shows that the gene was properly integrated in plants of lines 1, 2 and 10, but it is unclear whether the plants of lines 9 and 11 have the gene. Plants representing lines 3, 4 and 8 are unlikely to be transgenic. However, PCR can be unreliable, so further tests had to be performed to verify these results.

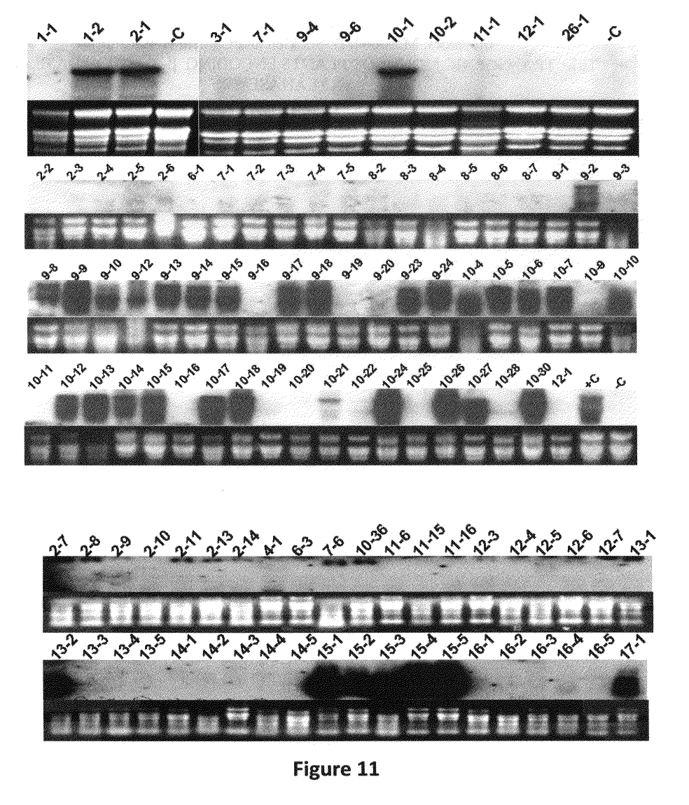

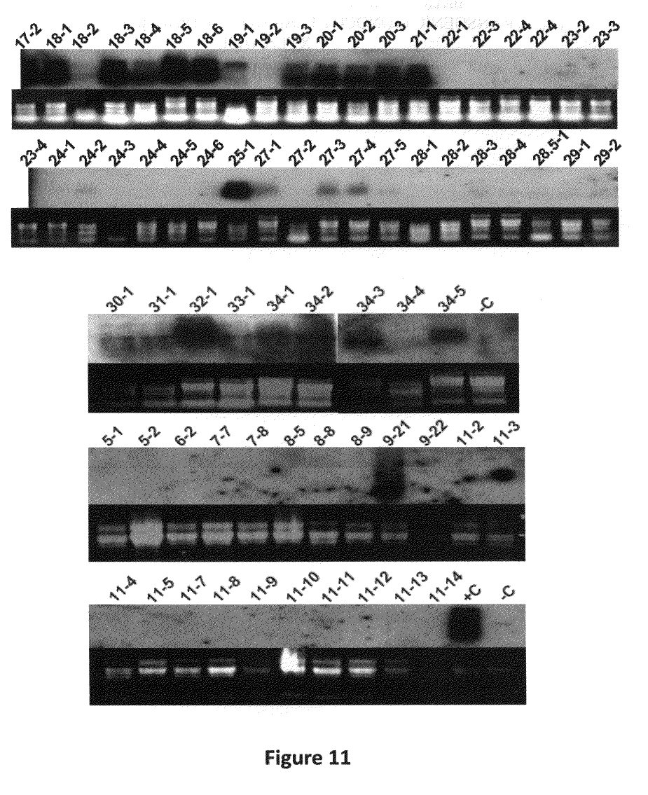

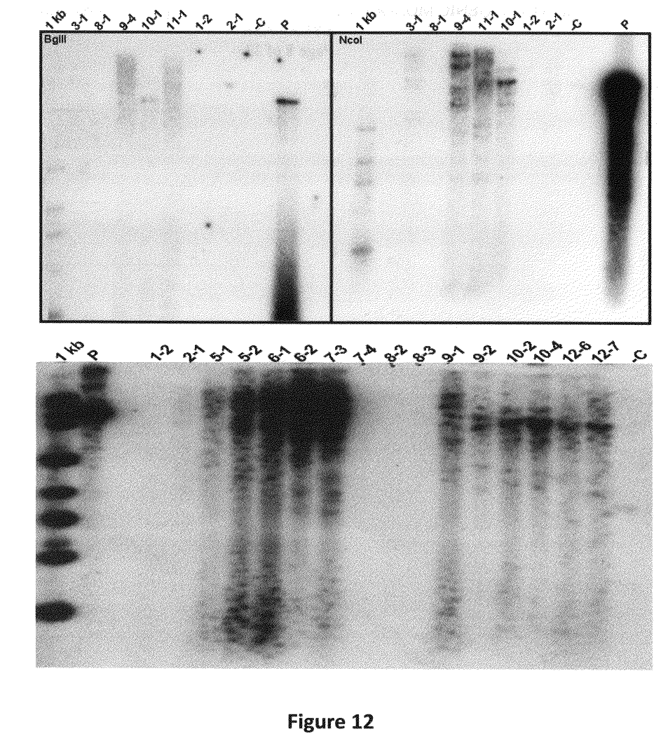

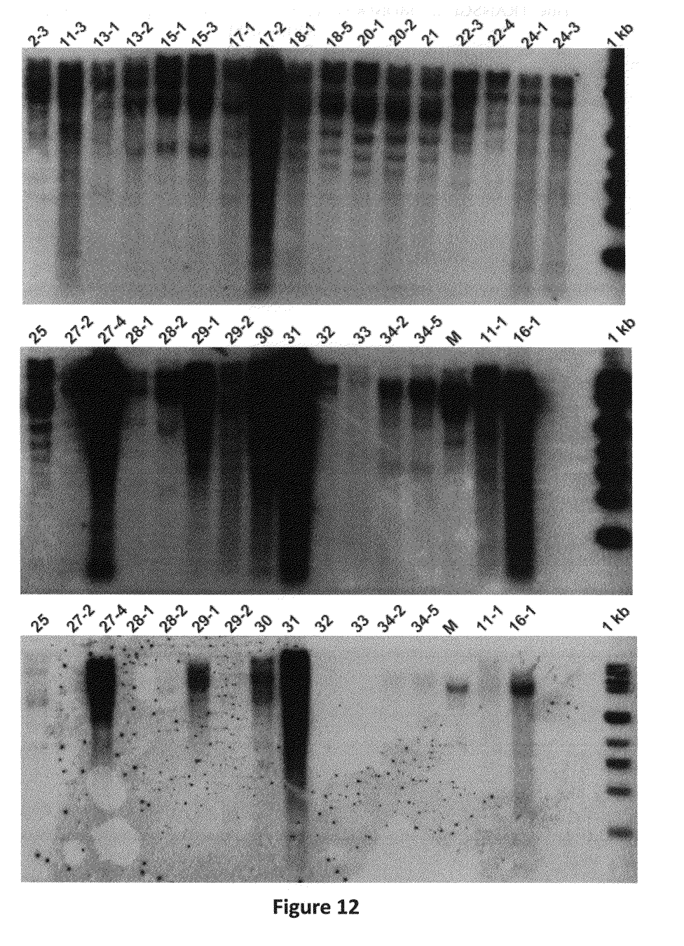

Northern blots for BG (FIG. 11) were performed on 12 lines (1, 2, 3, 5, 6, 7, 8, 9, 10, 11, 12, and 26). Ethidium-bromide-stained bands are shown below the blot to show relative amounts of RNA loaded on the gel prior to transfer; this indicates that there was reasonably even loading and thus differences in intensity of bands signify differences in expression. Hybridization with the BG probe was detected in lines 1, 2, 3, 9 and 10, indicating that these plants are producing BG RNA (i.e., expressing the gene). Plants 3-1 and 10-4 did not show amplification by PCR (FIG. 10), but 3-1 shows a very weak signal and 10-4 a strong signal on the northern blot (FIG. 11); 3-1 displays a weak signal on the Southern blot (FIG. 12; see next section). In addition, most plants (12 out of 19; 63%) of line 9 were expressing BG. PCR of plants from line 9 showed smaller than expected bands, and for two of the plants there were two bands (9-2, 9-4; FIG. 10). Plant 9-2 is expressing BG RNA; however, plant 9-4 showed the same pattern after PCR and yet was not expressing BG. As mentioned above, PCR is not always reliable and that is why it is important to do further testing.

Lines 1, 2, 9 and 10 had representative clones that also did not hybridize with the probe, suggesting somaclonal variation in the lines or silencing in these particular plants. Therefore it was necessary to screen all plants and not just representatives, to avoid missing any potentially superior lines.

Southern blots for BG (FIG. 12) were performed on 29 out of 34 lines (all except 4, 14, 19, 23 and 26). Hybridization with the BG probe was detected in all lines tested except line 8. Thirty micrograms of genomic DNA and 1 ng plasmid DNA (pUC1813) were digested overnight with either BglII (FIG. 12 A, left panel) or NcoI (FIG. 12 A, right panel). These were the two restriction enzymes to choose from that had a single cutting site in the construct and were methylation insensitive. Cutting with NcoI appeared to give clearer results and was used in the rest of the blots.

The blots confirm that these plants have the gene and that they are independent transgenic lines. Apparent copy numbers of the various lines is consolidated in Table 5. As mentioned in Materials and Methods, copy number was determined by counting the bands. Copy numbers are only an estimate; darker bands could represent more than one copy and the actual copy number may therefore be higher in plants with dark bands. For some plants (those in Panel B or those that were too dark), it was impossible to quantify copy number due to blot quality. As there were different numbers of bands and the bands were different sizes, the gene was integrated into different locations in the genome and the plants in fact represented independent transgenic lines. The DNA of lines 1 and 2 was undetectable in the agarose gel prior to transfer, indicating that the DNA quantification was inaccurate, and thus the amounts of DNA loaded were insufficient to enable measurable detection.

TABLE 5
Plant lines (T₀) and apparent copy numbers based
on bands on Southern blots.

| Line | Copies |
|---|---|
| 1 | 2 |
| 2 | 3 |
| 3 | 5 |
| 5 | ? |
| 6 | ? |
| 7 | ? |
| 8 | 0 |
| 9 | 5-6 |
| 10 | 4-5 |
| 11 | 6-7 |
| 12 | ? |
| 13 | 7 |
| 15 | 5 |
| 16 | 1 |
| 17 | 8 |
| 18 | 8 |
| 20 | 9 |
| 21 | 9 |
| 22 | 8-9 |
| 24 | 7 |
| 25 | 7 |
| 27 | 5+ |
| 28 | 6 |
| 29 | 5+ |
| 30 | 5 |
| 31 | ? |
| 32 | 2-3 |
| 33 | 1 |
| 34 | 4-5 |
| M | 4-5 |

3. TSP from Transgenic BG Plants Converts Cellobiose to Glucose

A preliminary glucose conversion activity assay for BG (Table 6) tested the plants' ability to degrade cellobiose. TSP (100 μg) from plants from lines 2, 3, 8, 9, 10, 11 and 26 was added to 0.5M citrate buffer, pH 4.8, with 0.015M cellobiose substrate. Most plants showed a decrease in glucose after the 30-min incubation at 50° C.; however, all differences were negligible and similar to negative control. A paired t-test indicated no significant difference between readings at 0 and 30 min. The standard protocol for determining cellobiase (β-glucosidase) activity of commercial enzymes (Ghose 1987) was followed.

TABLE 6
Glucose (g/L) released from 0.015M cellobiose
after 30 minutes using 100 μg maize TSP from plants
transformed with BG. HiII: Untransformed maize negative
control. The difference between 0 and 30 min was not
significant at α = 0.05 (t = −2.917, df = 12).

| Line-Plant | 0 min | 30 min | Difference |
|---|---|---|---|
| 2-1 | 0.111 | 0.109 | −0.007 |
| 2-2 | 0.113 | 0.117 | −0.014 |
| 2-4 | 0.085 | 0.088 | −0.018 |
| 3-1 | 0.085 | 0.078 | −0.014 |
| 8-1 | 0.100 | 0.086 | −0.01 |
| 9-4 | 0.081 | 0.063 | −0.005 |
| 9-6 | 0.084 | 0.070 | +0.002 |
| 10-1 | 0.117 | 0.107 | −0.012 |
| 10-2 | 0.007 | 0.065 | −0.015 |
| 10-3 | 0.073 | 0.075 | +0.004 |
| 11-1 | 0.101 | 0.089 | −0.002 |
| 26-1 | 0.081 | 0.066 | +0.004 |
| HiII | 0.072 | 0.076 | +0.003 |
| Mean | 0.090 | 0.084 | −0.006 |
| St. Error | 0.005 | 0.005 | +0.002 |

A second conversion experiment was carried out with a 5-ml reaction using 570 μl TSP, using the same protocol as above, scaled to 5 ml. In this experiment, the TSP was obtained from 1 g leaf tissue from RNA-positive plants and the resultant amount (mg) is indicated in Table 7. Plants showing high expression of BG in northern blots (FIG. 11) were chosen for this study.

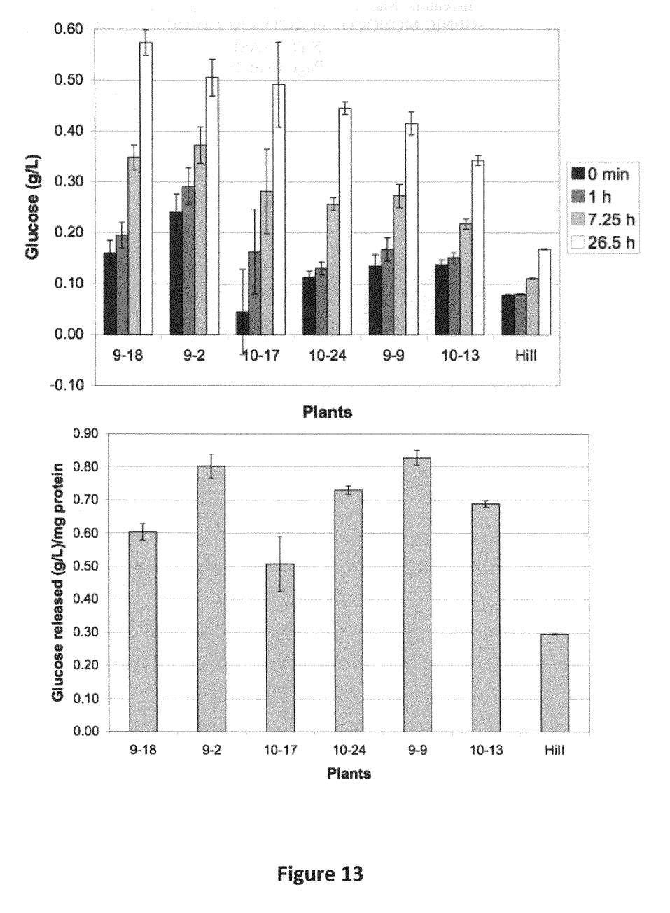

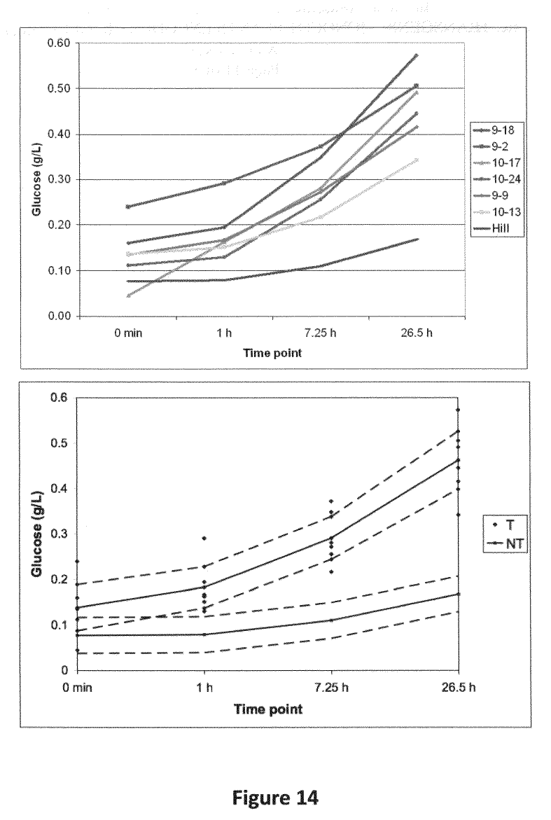

Samples were collected at several time points: 30 min, 1 h, 7.25 h and 26.5 h (Table 7, FIGS. 13 and 14). The data were adjusted by subtracting the substrate blank. After 30 min, the glucose released in most of the samples had decreased relative to 0 min (data not shown), similar to the above experiment. After 1 h, an increase in glucose was observed in most of the samples, but the differences were mostly negligible. After 7.25 h, differences in glucose release compared to 0 min were becoming substantial. These results show that 30 min is not enough time to see a conversion response; at least an hour is needed, and 6 or more hours will yield quantifiable results. The longer the reaction time, the higher the measured glucose release.

The increase in glucose release observed in plants ranged from 3.4 (plant 9-18) to 2.04 (plant 10-13) times higher than that of untransformed control. Glucose release above 0.4 g/L (4% conversion) was observed in 9-18, 9-2, 10-17, 10-24 and 9-9 after 26.5 h. The standard assay for unit calculation is standardized for a 4% conversion; thus the reaction (amount of TSP and time) must be optimized. This is an indirect method for measuring BG activity. The lower panel of FIG. 14 also shows relative performance of the various lines in terms of glucose released (g/L) per mg protein. Although 9-18 performed best, 9-9 and 9-2 actually displayed greater activity, followed closely by 10-24 and 10-13.

TABLE 7
Glucose (g/L) released from 1% cellobiose at 0 min,
1 h, 7.25 h and 26.5 h, using 570 μl maize TSP from
plants expressing BG. Data have been adjusted by
subtracting the substrate blank. HiII: Untransformed
maize negative control. The differences between 0 min
and 1 h, 0 min and 7.25 h, and 0 min and 26.5 h were all
significant at α = 0.05 (t = 2.667, 6.733 and 8.338
respectively, df = 6).

| | Total | | | | | Times |
|---|---|---|---|---|---|---|
| Line- | Protein | | | | | Higher |
| Plant | (mg) | 0 min | 1 h | 7.25 h | 26.5 h | than HiII |
| 9-18 | 0.951 | 0.16 | 0.20 | 0.35 | 0.57 | 3.41 |
| 9-2 | 0.630 | 0.24 | 0.29 | 0.37 | 0.51 | 3.01 |
| 10-17 | 0.968 | 0.05 | 0.16 | 0.28 | 0.49 | 2.92 |
| 10-24 | 0.610 | 0.11 | 0.13 | 0.26 | 0.45 | 2.65 |
| 9-9 | 0.502 | 0.14 | 0.17 | 0.27 | 0.42 | 2.47 |
| 10-13 | 0.497 | 0.14 | 0.15 | 0.22 | 0.34 | 2.04 |
| HiII | 0.570 | 0.08 | 0.08 | 0.11 | 0.17 | 1.00 |
| Mean | 0.675 | 0.13 | 0.17 | 0.27 | 0.42 | |
| St. | 0.076 | 0.024 | 0.022 | 0.022 | 0.03 | |
| Error | | | | | | |

BG enzyme activity was measured with the p-nitrophenol assay and percentage BG in TSP roughly estimated by comparing with Novozyme 188 (80% BG). The results are shown in Table 8, and show activities between 0.268 and 5.475 pNPU and 0.15-3.11% BG in TSP.

TABLE 8
Mean activity in units pNP (pNPU) and estimated
% BG in transgenic plant TSP. n = 3.

| Plant | pNPU | % BG | St. Dev. |
|---|---|---|---|
| 3-1 | 5.475 | 3.11 | 0.172 |
| 9-18 | 3.840 | 2.18 | 0.154 |
| 9-2 | 3.242 | 1.84 | 0.163 |
| 2-1 | 2.768 | 1.57 | 0.109 |
| 10-24 | 1.604 | 0.91 | 0.073 |
| 9-14 | 1.586 | 0.90 | 0.053 |
| 10-17 | 1.563 | 0.89 | 0.100 |
| 9-9 | 1.554 | 0.88 | 0.037 |
| 10-12 | 1.547 | 0.88 | 0.065 |
| 10-13 | 1.193 | 0.68 | 0.080 |
| 10-30 | 1.182 | 0.67 | 0.066 |
| 9-8 | 0.832 | 0.47 | 0.026 |
| 9-15 | 0.805 | 0.46 | 0.027 |
| 10-10 | 0.746 | 0.42 | 0.177 |
| 10-5 | 0.747 | 0.42 | 0.044 |
| 10-14 | 0.738 | 0.42 | 0.151 |
| 10-7 | 0.497 | 0.28 | 0.034 |
| 9-17 | 0.445 | 0.25 | 0.071 |
| 10-6 | 0.389 | 0.22 | 0.031 |
| 9-12 | 0.302 | 0.17 | 0.027 |
| 10-15 | 0.290 | 0.16 | 0.059 |
| 9-23 | 0.268 | 0.15 | 0.009 |

### V. Results of Experiments Related to Optimizing Ratios of Plant-Produced Hydrolysis Enzymes for Conversion

### 1. Introduction.

Enzymes used in the hydrolytic conversion of cellulosic biomass to fermentable sugars act synergistically (Hoshino et al. 1997), and certain ratios of endoglucanase, exoglucanase and β-glucosidase (among other enzymes) are considered ideal (Zhang and Lynd 2006; Boisset et al. 2001). Commercial cellulose mixtures are often produced from fungal or bacterial culture media and thus have differing enzyme ratios depending on the mix that particular species produces (Kabel et al. 2006). Companies such as Genencor are working on optimizing enzymatic hydrolysis by determining the best available enzyme of each class produced out of all species (and at times enhancing this activity even further by creating synthetic genes), and optimizing the ratios of these enhanced enzymes for maximum sugar production.

Although this is useful for bacterial or fungal enzyme production, plant-produced enzymes may not behave as expected. Enzyme production is likely to be lower as a percentage of total soluble protein (TSP) in plants and activity may not be as high as in commercial enzyme preparations; this could be due to many factors, including low expression, lack of enzyme purity, glycosylation, truncation or incorrect folding. Therefore, it is important to test plant-produced enzyme activity and optimize ratios of TSP necessary for maximum conversion.

Endoglucanase (E1) and exoglucanase (CBHI) were produced in maize and tobacco by Dr. Chuansheng Mei. Several lines of each have been tested and the activities measured by MUCase assay and conversion experiments (described in Materials and Methods). Four were considered the best (i.e., had the most activity); three E1 plants and one CBHI plant. The TSP of these plants was extracted and used in the following experiments. β-glucosidase (BG) was produced in maize and its activity confirmed indirectly with the IUPAC method and conversion assays, and directly with the pNPPG assay (described in Materials and Methods and presented in BG Results).

In this set of experiments, the plant TSP containing the hydrolysis enzymes was tested alone and in combinations to determine the best mixture to use for biomass conversion. It was found that the ratio of E1:CBH1:BG 1:4:1 worked best for converting 1% AFEX-treated corn stover to fermentable sugars, and this worked as well as using commercial Spezyme CP plus plant TSP containing BG.

Materials and Methods for this chapter are listed in the main Materials and Methods section (Pages 56 and 59).

### 2. Results and Discussion

### 2.1. Preliminary Results

### 2.1.1. Commercial Enzyme Test Plates

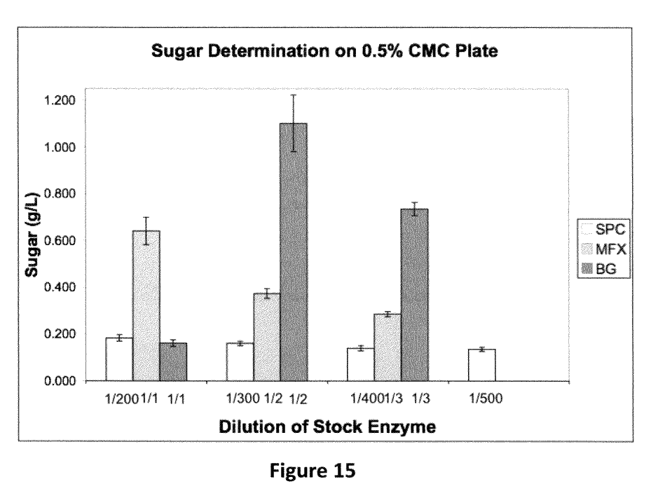

First, commercial enzyme preparations of Spezyme CP (a commercial cellulase mixture containing mostly endo- and exo-glucanases), multifactorial xylanase and Novozyme 188 (a commercial cellulase mixture containing roughly 80% β-glucosidase) were tested on a 0.5% CMC plate to test the method (FIG. 15).

The Spezyme dilutions (1:200, 1:300, 1:400 and 1:500) were below 0.2 g/L, and there was a steady decrease. Multifactorial xylanase also showed a decreasing trend, and at the 1/3 dilution, was getting close to 0.2 g/L. Undiluted Novozyme 188 showed under 0.2 g/L, 1/2 dilution was over 1 g/L and 1/3 was over 0.7 g/L. The results were not as expected (FIG. 15). The low glucose release of the undiluted enzyme could be either due to inhibition due to excess enzyme or viscosity that may have caused inaccurate pipetting.

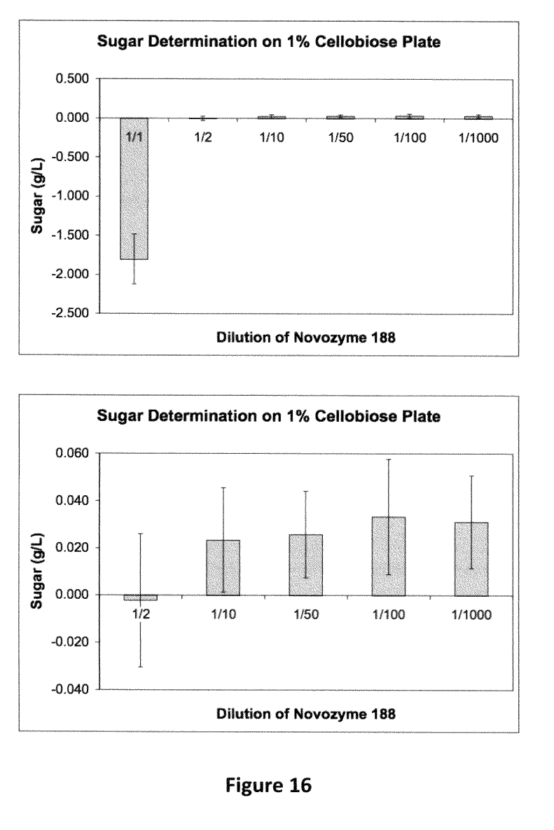

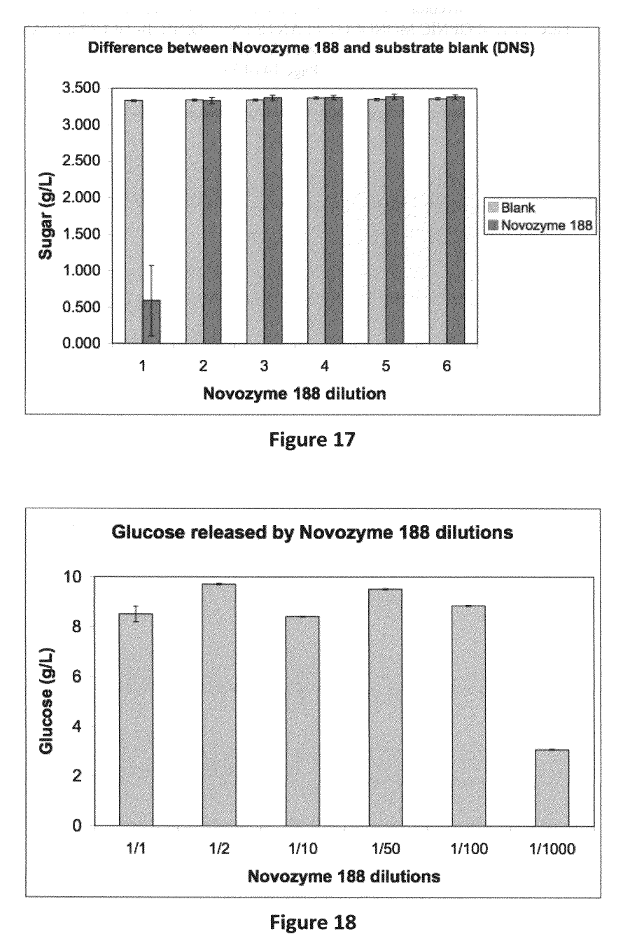

To test the suitability of the microplate DNS assay for β-glucosidase on cellobiose, its native substrate, a second assay was performed using Novozyme 188, a commercial cellulase mixture that is mostly (around 80%) β-glucosidase (FIGS. 16 and 17).

The readings were very low after subtracting the blank (FIG. 16). It was found that DNS cannot be used for cellobiose, which is itself a reducing sugar, because the background is too high for detection of increased sugars (FIG. 17): almost 3.5 g/L glucose, which is nearly 10 times the amount required (4%) for proper determination via this assay.

To determine if it would be possible to use microplate hydrolysis for Novozyme 188 before DNS color development, samples from the plates after the hydrolysis step (which had been kept at 4° C.) were analyzed for glucose. FIG. 18 shows that there is no trend and the numbers fluctuate around the same amount g/L) in dilutions 1/1 to 1/100. At 1/1000, however, the numbers make a dramatic drop, down to 3 g/L, indicating that dilutions of Novozyme 188 should start at this point and go down from here to achieve the dilution necessary for 0.4 g/L.

### 2.1.2. Plant-Produced E1 and CBH1

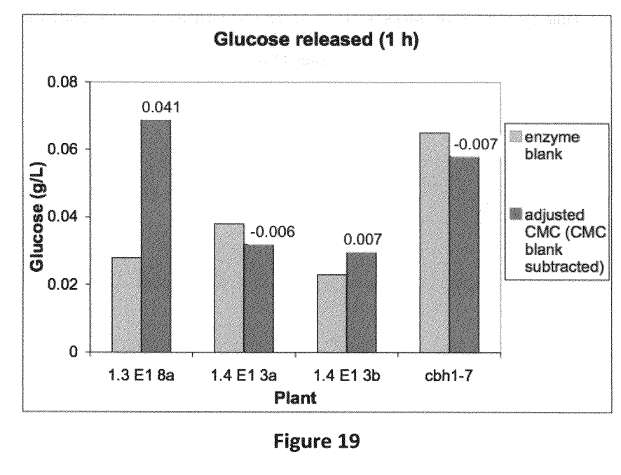

Dr. Chuansheng Mei had previously determined that four plants out of the ones he tested showed the highest enzyme activity. Total soluble protein (TSP) of these plants expressing E1 or CBHI was tested on a 1% CMC plate and, to determine if the plant-produced enzymes had any activity after 1 hr hydrolysis, samples from the plates were analyzed for glucose. The readings were very low, the highest being not quite 0.07 g/L, much lower than the needed 0.4 g/L (FIG. 19). Half the readings were higher and half lower than the enzyme blanks.

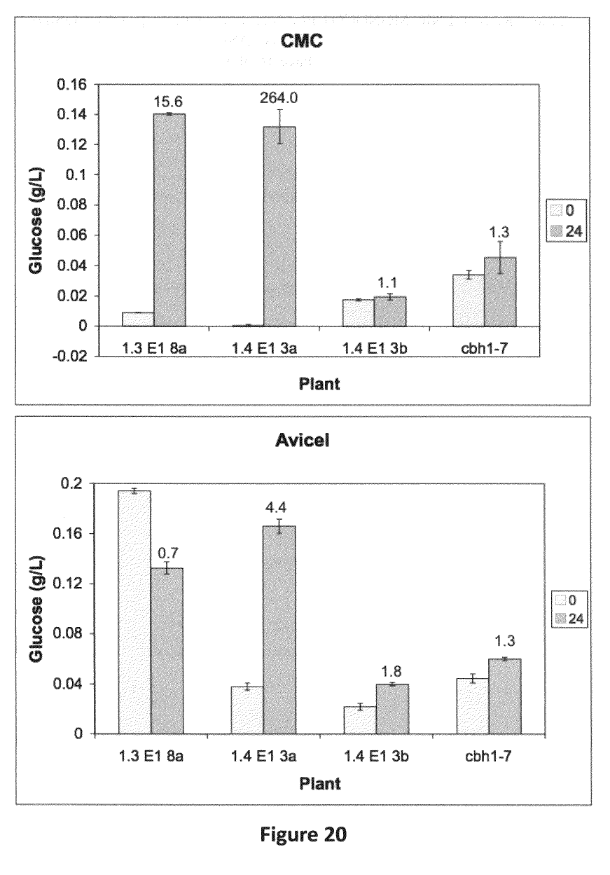

Since the results of the 1-h hydrolysis were inconclusive, a longer reaction was attempted. This time, plant TSP was incubated in 1% CMC or Avicel for 24 h at 50° C. and 90 rpm; a 0 time point was also taken. The results (FIG. 20), determined via glucose analyzer, showed that two of the plants' TSP definitely had activity on CMC (1.3 E1 8a and 1.4 E1 3a); 1.3 E1 8a was used in subsequent studies.

The other two had increases after 24 h that were negligible. Regardless, the conversion did not reach even half of the needed 0.4 g/L. On Avicel, the results were not encouraging either; while the release was nearly the same as for CMC, 1.3 E1 8a was considerably less after 24 h than 0 min. The TSP was quite concentrated as well: a total of 273 μg 1.3 E1 8a, 463.6 μg 1.4 E1 3a, 501.6 μg 1.4 E1 3b and 486.4 μg CBHI-7 TSP were used. In comparison, five out of six of the plants expressing BG had exceeded 0.4 g/L after 24 h on cellobiose with 500-950 μg TSP.

The assay is an initial rate assay and designed to allow calculation of enzyme activity after only 30 min hydrolysis. Since the plant TSP needs 6-24 h hydrolysis, and the longer the better, the assay cannot be used for direct calculation of enzyme activity. However, it can be used for measuring sugars released during hydrolysis. In this case, it is not necessary to reach 0.4 g/L; any amount is acceptable, since the assay will not be used to calculate activity, only to measure sugar released.

### 2.2. Optimal Concentrations of Transgenic Plant TSP

Another researcher determined that the plant-produced E1 and CBHI described above work synergistically together best in a ratio of 1:4. The next step was to determine the relative amount of plant-produced BG to add to this fixed ratio that would give the best conversion on both pure cellulose and AFEX-treated corn stover. To this end, BG plant TSP was tested for ability to convert Avicel and CMC, and, keeping the ratio of E1 and CBH1 1:4, varying amounts of BG (in terms of percentage of the total of E1 plus CBHI) were added to determine the optimal balance.

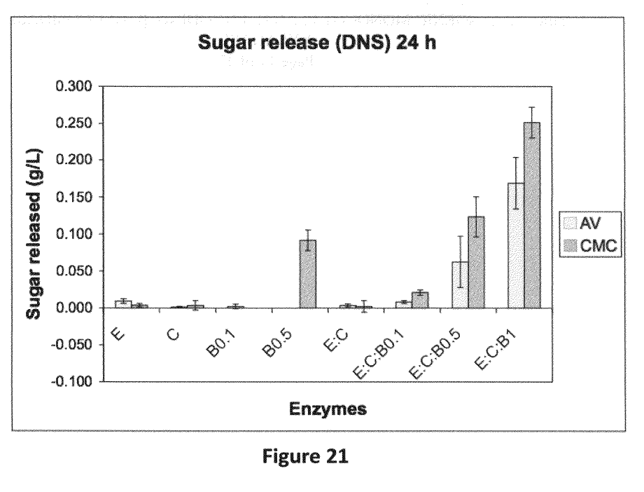

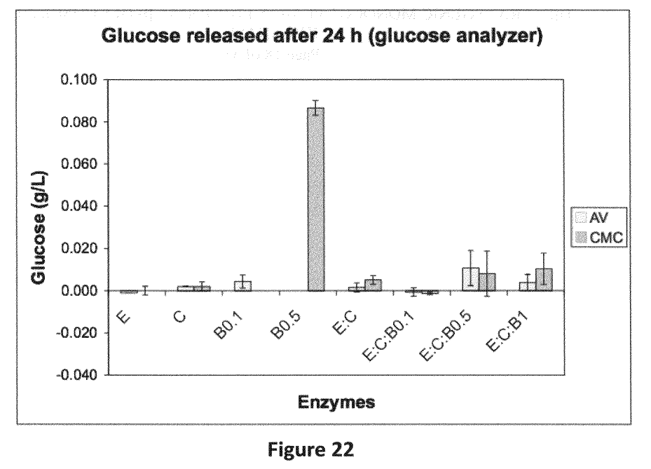

First, enzymes E1 and CBH1 were concentrated using a centrifugal concentrator to a concentration of 4.34 and 5.71 μg/μl respectively. Then, a 24-hour hydrolysis reaction was done on 1% CMC and 1% Avicel using a 1:4 ratio of E1 to CBH1 and varying amounts of BG relative to E1:CBH1: 0.1, 0.5 and 1. Hydrolysis was performed at 50° C., 90 RPM in a total reaction volume of 10 ml. The amounts used were limited by the amount of plant TSP available. In this experiment, 30 μg E1, 120 μg CBH1, and 15, 75 and 150 μg BG were used. The blanks included were also substrate blank, E1 alone (on substrate), CBH1 alone (on substrate), BG0.1 (15 μg) alone (on Avicel), and BG0.5 (75 μg) alone (on CMC). Three replicates of each sample were included. More TSP blanks were not possible due to lack of TSP. Sugar release was measured after 24 hours with a DNS assay (FIG. 21) and with the glucose analyzer (FIG. 22).

Some general trends can be seen. For the most part, E1 and CBH1 alone and together do not have much activity, releasing below 0.01 g/L sugar after 24 hours (FIG. 21), most of which does not appear to be glucose (FIG. 22). BG0.1 alone and in combination with E1 and CBH1 is about the same. BG0.5 released much more sugar alone on CMC than BG0.1 on Avicel; this could indicate endoglucanase activity. However, this amount was more than when combined with E1 and CBH1, so it could be an anomaly, or it could be due to inhibition when combined with E1 and CBH1. Also, we see that as the amount of BG increases, so does the sugar. This could be due to free sugars present in he TSP, but since TSP blanks without substrates had not been included, it could not be verified. The sugar increases after 24 h on both substrates were more pronounced when determined via DNS (FIG. 21) than with the glucose analyzer (FIG. 22), so these free sugars are likely not glucose.

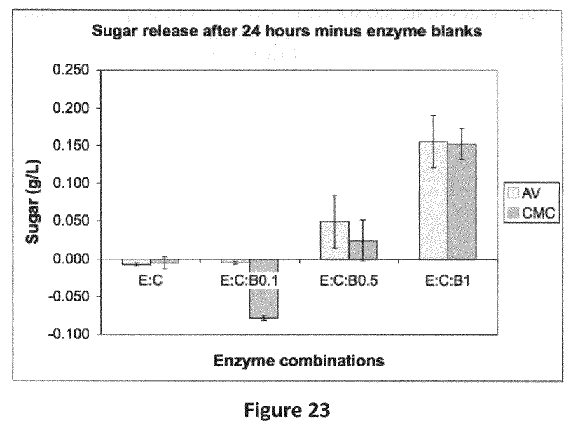

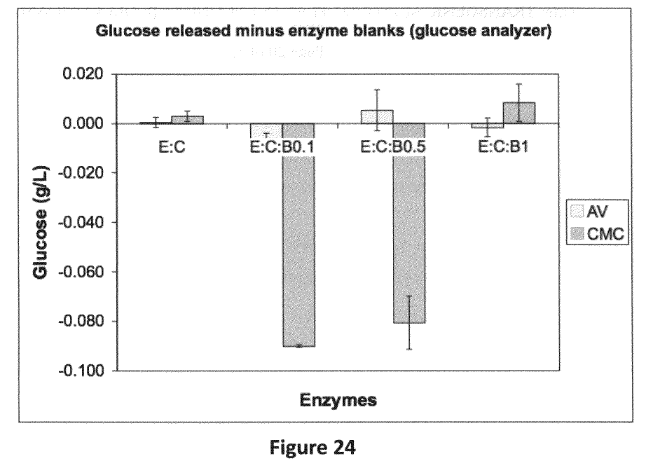

To get a better idea of how much increase was seen, the blanks were subtracted from the 24-h data (FIGS. 23 and 24). Please note that because BG blanks included only BG0.1 on Avicel and BG0.5 on CMC (due to lack of TSP), the BG0.1 blank was used for E:C:BG0.5 and E:C:BG1 on Avicel, and the BG0.5 blank was used for E:C:BG0.1 and E:C:BG1 on CMC. So in the case of Avicel, the E:C:BG0.5 amount is likely lower than shown; in the case of CMC, the E:C:BG0.1 amount is likely higher than shown; and in the case of both substrates, the E:C:BG1 amount is likely lower than shown.

After subtracting the blanks, it is clear that glucose release is very low, even with the highest relative concentration of BG. Total sugars are also low (around 0.15 g/L). Many of the readings were also negative.

Due to the many problems with this experiment, a second reaction was done. The BG and E1 TSP had been used in the previous experiment, and the (maize) plants had since dried down, so TSP was extracted from leaf tissue from entire plants. Although E1 from dried material had been shown to be active in a previous study, this E1 was the result of a different transformation event and subcellular localization (ER vs. apoplast), and it was unknown whether it or BG would maintain activity after dry-down.

In this experiment, E1, CBH1 and BG were concentrated to 4.9 μg/μl, 6.6 μg/μl and 5 μg/μl respectively. A 1:4 ratio of E1 to CBH1 was maintained, and 1:0.1, 1:0.5, 1:1, and 1:2 ratios of E1:CBH1:BG were tested on both 1% Avicel and CMC. As before, TSP availability determined amount used; 4.6 μg E1, 18.2 μg CBH1, and 2.3, 11.4, 22.8 and 45.5 μg BG were used in a total volume of 500 μl. Three replicates were prepared for every sample, and data were adjusted by subtracting the substrate blanks. Hydrolysis was performed at 50° C., 350 RPM. All analyses were performed via DNS assays and a single timepoint of 18 h was used. Blanks included substrate, enzyme alone in water and enzyme alone in substrate.

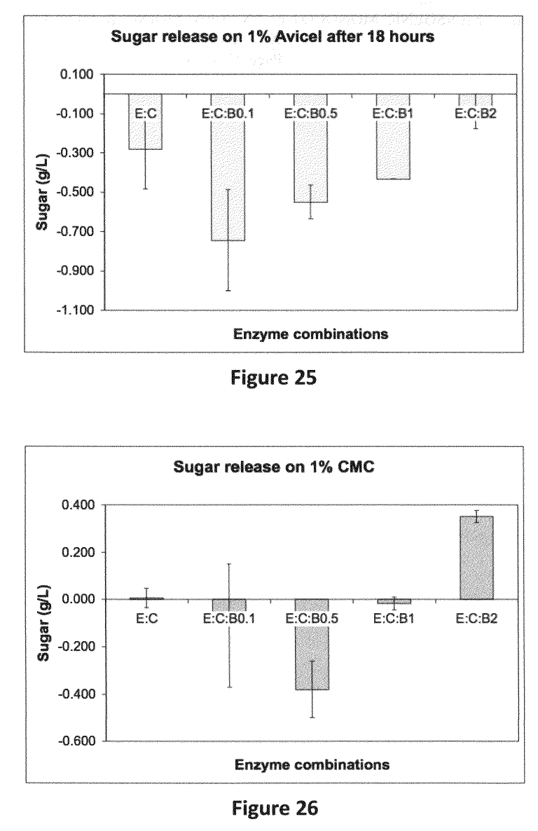

Sugar released after subtracting the blanks is shown in FIG. 25 for 1% Avicel and FIG. 26 for 1% CMC. On Avicel, the plant TSP is not contributing at all to the conversion to sugar, as the values are all negative. The amount of free sugars attributable to TSP decreases as the amount of BG increases, however, as the numbers become less negative. This could indicate some BG activity. (On Avicel, only one replicate of E:C:B1 and two replicates of E:C:B2 were readable by the plate reader.) On CMC, the results are similar in that they are all negative except for BG2. BG2 shows a release of around 0.35 g/L, nearly 4%. Perhaps a higher ratio of BG to E1:CBH1 is needed to realize maximum conversion. It appears that the plant-produced E1 and CBH1 have very little, if any, activity.

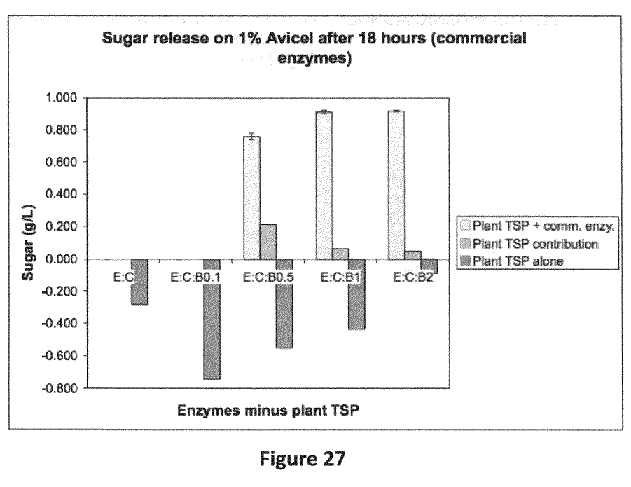

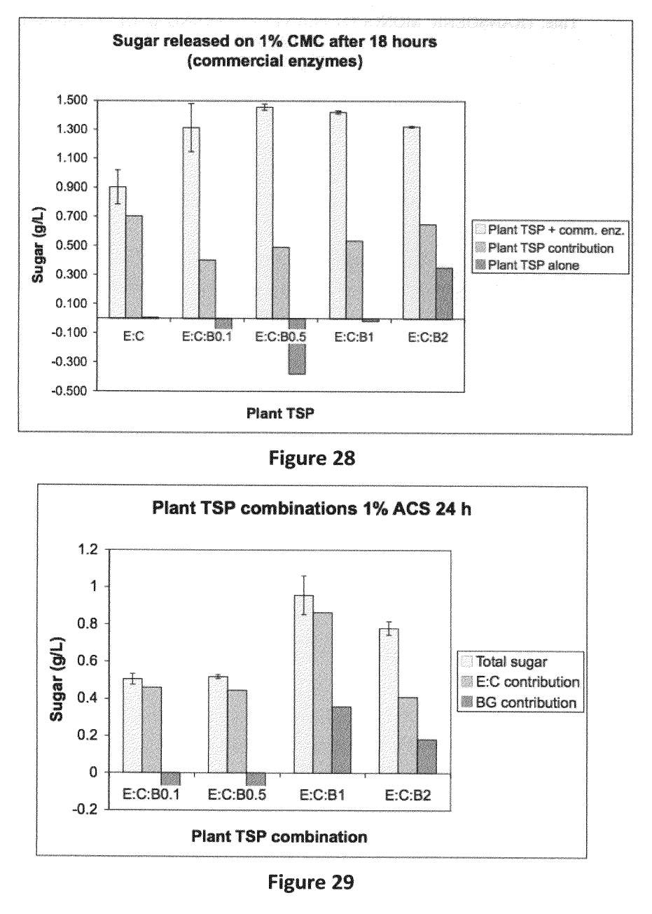

The plant TSP was also tested for its ability to substitute for commercial enzymes. Novozyme 188 was added to plant TSP containing E1 and CBH1 at the same rate as used in normal conversion experiments using commercial enzymes, scaled to 500 μl. In addition, the various amounts of plant TSP containing BG were added to the normal amount of Spezyme CP, scaled to 500 μl. The results are shown in FIGS. 27 and 28. On Avicel, results were unreadable for E1:CBH1:Novozyme 188 and Spezyme CP:B0.1, and only two replicates were readable for Spezyme CP:B0.5. For this and subsequent analyses, the contribution of a particular enzyme in a combination was determined by subtracting the sugar released by the other enzyme(s) from the combined total sugar released. For example, in a combination of E1:CBH1 (1:4), to determine the amount attributable to E1, the amount of sugar released by CBH1 alone would be subtracted from the amount of sugar released by the combination of E1:CBH1. Obviously, this is only an estimate, and the amounts attributable to individual enzymes will not add up to the amount released by the combinations due to interactions (either synergy or inhibition or more complex interactions) or other factors, such as sample variation.

On Avicel, very little of the sugar is attributable to the plant TSP; however, this amount is more than the combined plant TSP alone on Avicel (same data as in FIG. 25). The amount of plant TSP contribution seems to decrease with the amount of BG added, which is opposite the results of the experiment with combined plant TSP. On CMC (FIG. 28), a comparatively larger fraction of the total sugar release is attributable to plant TSP. These results are also for the most part inconsistent with the combined plant TSP results (same data as FIG. 26), as they are mostly negative; the only exception again is BG2. However, the amount of plant TSP contribution seems to increase with the amount of BG added, consistent with the experiment with combined plant TSP. This could again indicate some endoglucanase activity.

The ultimate goal of producing hydrolysis enzymes in plants is to use them in actual biomass conversion. Therefore, combinations of plant-produced E1, CBH1 and BG were applied to AFEX-treated corn stover representing 1% glucose in a 24-hour hydrolysis reaction. The ACS had been ground to a fine powder prior to AFEX treatment to allow it to be mixed and treated as a slurry. As before, enzyme and substrate blanks were included and all reactions were done in triplicate. Hydrolysis was performed at 50° C., 350 RPM in a total volume of 750 μl. Sugar release was determined via DNS assay.

Sugar release from combined plant TSP after subtracting blanks is shown in FIG. 29. The results show a much higher sugar release than for either CMC or Avicel. The best combination tested appears to be a 1:4:1 combination of E1:CBH1:BG, with release of nearly 1 g/L. Most of this amount appears to be due to E1 and CBH1, but for this ratio it also has the highest BG contribution. In fact, for all of the ratios tested, E1 and CBH1 account for most of the sugar release seen, and BG contributes relatively little or none at all.

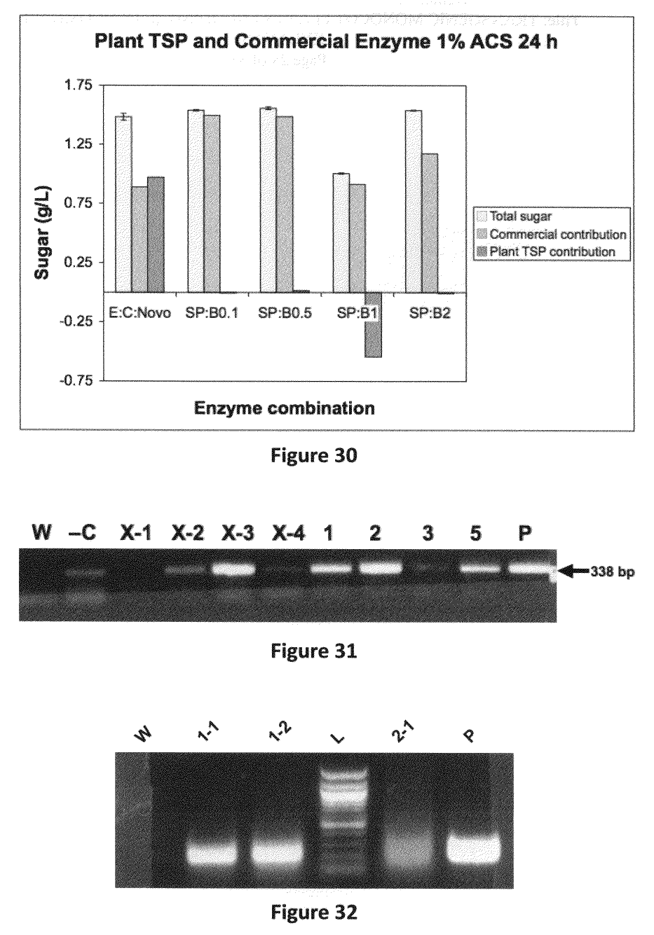

The same pattern is present when plant TSP is compared with commercial enzyme (FIG. 30). When Novozyme 188 is added to E1:CBH1 (1:4), its contribution to the sugar release is less than the amount attributable to plant TSP. However, plant BG TSP does not contribute much, if anything, to the sugar release.

### VI. Discussion

### Plant-Produced E1 and BG

Expression of the E1 catalytic domain yielded more activity than the full-length enzyme (Ziegelhoffer et al. 2001); therefore it was chosen for the work in maize (Biswas et al. 2006; Ransom et al. 2007). The expression cassette contained the strong constitutive cauliflower mosaic virus (CaMV) 35S promoter, the tobacco mosaic virus (TMV) Ω translational enhancer, the apoplast-targeting tobacco Pr1a signal peptide, and the polyadenylation signal of nopaline synthase. With the same cassette in Arabidopsis, the plants produced E1 at a range of 0.01 to 25.7% of TSP (Ziegler et al. 2000). In the work with maize (Ransom et al. 2007), the estimated E1 protein accumulation was up to 2.1% of TSP. Although this is several fold lower than reported in Arabidopsis (Ziegler et al. 2000), it is in the range reported in transgenic tobacco (Dai et al. 2005) and potato (Dai et al. 2000b). Due to the random nature of transformation, expression levels can vary due to position effects. Screening a larger number of tranformants could yield maize lines with higher activity.

The expression cassette for BG included the CaMV 35S promoter, an ER-leading sequence at the 5′ end of the bglA gene, a vacuole-targeting sequence at the 3′ end, and the CaMV 35S terminator. In tobacco, this cassette was used to study increased SAR from β-glucosidase-induced conversion of SA 2-O-β-D-glucoside (GSA) stored in the vacuole to free salicylic acid (SA) (Yao 2004). In the work in maize (Chapter IV), it was useful to test the vacuole's capacity as a storage location for heterologous hydrolysis enzymes.

The maize-produced transgenic TSP containing E1 successfully hydrolyzed AFEX-treated corn stover and yielded glucose in conversion analyses after addition of commercial β-glucosidase. This was true even when extracted from tissue that was dry or stored several months in the freezer (Ransom et al. 2007). TSP containing BG was also able to hydrolyze cellobiose to glucose. Although the enzymes were biologically active, they did not hydrolyze cell walls in planta. There are several possible explanations for this. First, the E1 enzyme is thermostable and is most active at higher temperatures; its activity assay was performed at 65° C. This is much higher than ambient temperatures or even those experienced in summer field conditions. So, the enzyme may have had activity that was too low to damage the cell wall before heating. Testing this hypothesis with developing plants, however, may lead to detrimental consequences, such as heat damage to the plant and/or plant death. A second explanation is that the enzyme may be barred from its substrate because of its location in the apoplast or vacuole and because the cellulose itself is located inside hemicellulose and lignin. Third, while endoglucanase alone could cleave random internal bonds in the crystalline cellulose, it would require cellobiohydrolase and β-glucosidase to complete the hydrolysis. β-glucosidase alone could hydrolyze cellobiose units, but these are likely scarce without the prior activity of endo- and exo-glucanases. For these reasons it is safe to assume that growing individual hydrolysis enzymes in plants, targeted to cellular compartments, and especially if they are thermostable, poses no danger to cell wall integrity or development of the plant, as long as transformation does not cause mutations that adversely affect cell wall integrity or plant development, nor the transgene itself mutate to affect the same.

In the experiments for optimization of plant-produced cellulase combinations, the highest sugar release (a little over 1.5 g/L) was observed on 1% ACS after 24 h with SPC and BG0.5 (FIG. 30), but all of the reactions except SPC:B1 achieved around the same sugar release. The same was true for 1% CMC after 18 h; the release was nearly 1.5 g/L; SP:B0.5 was again the highest, but all the other combinations of SP:Bx were similar and above around 1.25 g/L (FIG. 28). The next highest was on 1% ACS with E:C:B1, nearly 1 g/L (FIG. 29). Nearly as high was SP:Bx on 1% Avicel after 18 h (FIG. 27), with amounts ranging from above 0.7 g/L (SP:B0.5) to around 0.9 g/L (SP:B1 and SP:B2).

On 1% ACS, combinations of plant produced enzymes appear to be nearly as effective as combinations that include SPC, although not as effective as the combination of SPC and Novozyme 188 on 1% ACS, which routinely generates 7-10 g/L sugar (data not shown). Plant-produced TSP on pure cellulose yielded disappointing results. It is unclear why sugar release should be so much greater on 1% ACS rather than pure cellulose with the same TSP.

Plant E1 and CBH1 did not appear to have much activity in preliminary assays, whereas BG appeared to have much more activity; however, in combinations, it was the E1 and CBH1 that appeared to have the most effect on sugar release rather than the BG. One explanation is that much more BG was used in preliminary experiments and not much was available for further experiments. Another issue is that in preliminary experiments using plant TSP, nearly 6.5 times the amount used in subsequent experiments was used, yet the sugar release was much greater for 1% ACS, although lower for pure cellulose. This is not a direct comparison, as the larger amounts were not available for use on 1% ACS.

In the future, the individual enzymes should be purified from the plant TSP if possible, to compare directly with commercial enzymes and test activity directly in comparison with purified enzymes. Although plant TSP contains a percentage of enzyme, it is impossible to know if this enzyme maintains its total activity or if other proteins or substances in the TSP affect its activity. Despite the drawbacks, it can be concluded that plant TSP can be used successfully on 1% ACS to achieve conversion, and may enhance the activity of commercial enzymes. However, the conversion is not as high as with commercial enzymes.

### 2. Conclusion

These experiments confirm that it is possible to grow hydrolysis enzymes in crop plants when targeted to subcellular compartments. The enzymes retain biological activity, are robust and storable, are able to convert pretreated feedstock biomass to fermentable sugars, and work in synergy. This is a step forward in the quest for alternatives to current enzyme production methods.

### Appendix A. Results for FLC

### 1. Introduction

FLOWERING LOCUS C (FLC) is a gene characterized in Arabidopsis that maintains a vegetative state until it is down-regulated by vernalization, allowing flowering to occur (Michaels and Amasino 1999; Sheldon et al. 1999). It was proposed that it could delay flowering in other plants and this hypothesis was tested and verified in tobacco (Salehi et al. 2005). This is an important characteristic to employ for both increasing biomass for biofarming or feedstock for ethanol production and for transgene containment. If plants with transgenes flower later than other plants of the same species in the surrounding fields, it is less likely that they will cross.

It was with these goals in mind that FLC (pgreen) was used in co-transformation with XYL1 and CBHI (pMSF15); pgreen also contains the bar gene to provide Bialaphos herbicide resistance as a selectable marker.

### 2. Materials and Methods

pGreen (FIG. 5) was used in co-transformation with pUC1813, XYL1 and pSMF15 as described on page 50.

For PCR, the following set of primers was used: FLC F: 5′-CGA TAA CCT GGT CAA GAT CC-3′ (SEQ ID NO: 7) (forward primer) and FLC R, 5′-CTG CTC CCA CAT GAT GAT TA-3′ (SEQ ID NO: 8) (reverse primer; Salehi et al. 2005). The predicted size of the amplified DNA fragments of the transgene was 338 bp. The PCR profile had an initial denaturation step at 94° C. for 1 min, followed by 30 cycles of 1 min at 94° C. (denaturation), 2 min at 60° C. (annealing) and 3 min at 72° C. (extension).

For Southern and northern analyses, a probe was generated by digesting pGreen plasmid DNA with XhoI and SpeI to release a 0.59-kb fragment containing the FLC coding region. Labeling and detection were done using non-radioactive methods described on page 53.

### 3. Results and Discussion

Maize plants bombarded with a mixture of pGreen and XYL1, pMSF15 or pUC1813 were tested via PCR for presence of the FLC transgene (FIGS. 31 and 32). In FIG. 31, all of the plants tested amplified a band of the correct size with the exception of X-1. Plants X-4 and 3 amplified a weak band. This result is not valid, however, due to the presence of a weak amplification product in the nontransgenic negative control. In FIG. 32, all three plants amplified the correct size band; plant 2-1 however was not as clear. The rest of the BG plants must also be tested for FLC, and analyzed using Southerns and northerns as well.

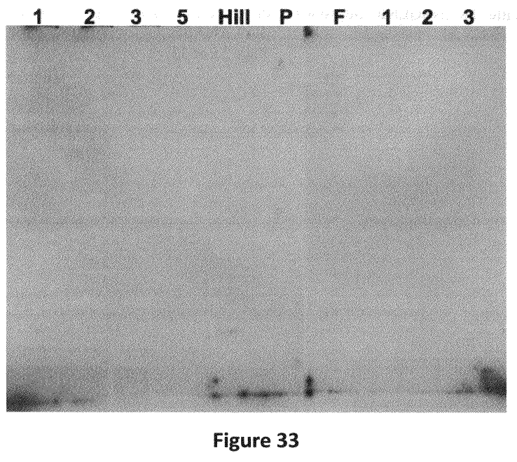

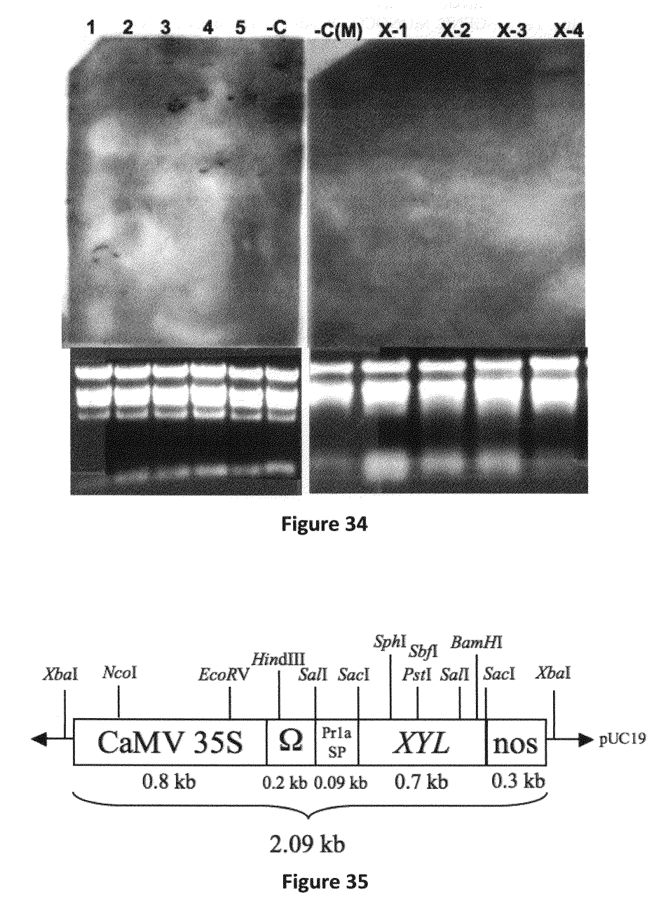

Southern and northern blots of maize co-transformed with either XYL1 or pSMF15 that had been used in hybridizations with their respective probes were stripped and probed using the FLC probe (FIGS. 33 and 34). No hybridization was detected, even with the pGreen plasmid DNA.

pGreen is a binary vector. As such, it was difficult to obtain enough DNA for bombardment. A project was commenced to attempt to clone the FLC construct into a simple vector. This was difficult because our plasmid map was incomplete and we did not have the complete sequence or cutting sites. Cloning was begun but proved fruitless; because the cloning site in pGreen is based on PBLUESCRIPTKS+, and this was the only cloning vector with the appropriate restriction sites (pGreen was cut with BglII to release the T-DNA, and pBSKS+ cut with EcoRI and treated with Klenow for a blunt-end ligation), it was impossible to check the sequence and very difficult to find unique sites for cloning. The project was therefore abandoned.

Another project related to FLC was the attempt to obtain a polyclonal antibody for use in western analyses. A synthetic protein sequence was generated by the Michigan State University Research Technology Support Facility. This was used as an antigen and sent to Cocalico Biologicals. They sent us prebleeds from five rats and a western was performed as described on page 57. Twenty micrograms genomic DNA (HiII) and 50 ng antigen were run in five lanes each; the blot was cut after blotting and each strip hybridized with a different prebleed diluted 1:100 in 1×PBS. Anti-rat:HRP was diluted 1:10,000 1×PBS for the secondary antibody. There was no background detected. Thereafter, they conjugated the antigen and injected the rats, and sent several bleeds, all of which were tested in a similar manner and all of which showed no response to the antigen. The antigen was then conjugated with a different carrier, thyroglobulin, and injected as before; however the response was still the same. The project is at a standstill as of now.

Appendix B. Xylanase Transformation of Maize (Along with FLC), Analyses of Tobacco and Maize Transformed with XYL1, and Cloning of XYL1 and a New XYL

### 1. Introduction

The XYL1 gene was cloned in Dr. Jonathan Walton's laboratory from the fungus Cochliobolus carbonum. Previously in the Walton lab, Apel (1996) used XYL1 to transform tobacco using two constructs (not in the same plants): one with the native fungal signal peptide and one without. Both were not targeted to any cellular compartment and so remained in the cytoplasm. She was not successful in finding expression via Western blotting although she had some promising northern blots.

### 2. Materials and Methods

XYL1 with native fungal signal peptide was sent to a company, Norclone, to put into the apoplast-targeting vector pMZ766 in place of the E1 gene (FIG. 35). This plasmid was used for tobacco transformation (performed by another researcher) and particle bombardment, along with pGreen (FIG. 5), of maize.

Maize callus production, bombardment, selection and regeneration were carried out as described previously in Materials and Methods (page 50), as were DNA, RNA and protein extraction (pages 51, 53 and 54) and Southern, northern and western blotting (pages 53 and 57). For Southern blots, 15 μg genomic DNA was digested overnight with HindIII and fractionated on a 1% agarose gel. Tobacco that had been transformed with Agrobacterium by another researcher were included as well. PCR and RT-PCR were performed using the following primers and conditions: Xyl1-F: 5′CTG CCC GTA CCA TCA CCT AC 3′ (SEQ ID NO: 9) and Xyl1-R: 5′GTG ATC TGG GCG TTA CCA GT 3′ (SEQ ID NO: 10) (397 bp); 94° C. for 3 min; 35 cycles of 94° C. for 45 s, 56° C. for 45 s, 72° C. for 45 s; 72° C. for 10 min. The probe used for Southern and northern hybridization and detection was the 397-bp fragment generated by PCR described above.

A polyclonal antibody to XYL1 was obtained from Dr. Walton's laboratory and used in western blots. For activity assays, the assay for reducing sugars using PABAH (Lever 1972) was used. To a 1% oat spelt xylan substrate, 25 μl protein samples were added to 0.5M acetate buffer in a total volume of 300 μl, and incubated at 37° C. for 0 min, 30 min, 4 h and overnight. Samples (25 μl) were collected at the designated time points, 1.5 ml PABA solution was added, the samples were mixed, heated at 100° C. for 10 min and cooled before reading absorbance at 410 nm.

### 3. Results and Discussion

3.1 Transformation of Maize with XYL1 and FLC

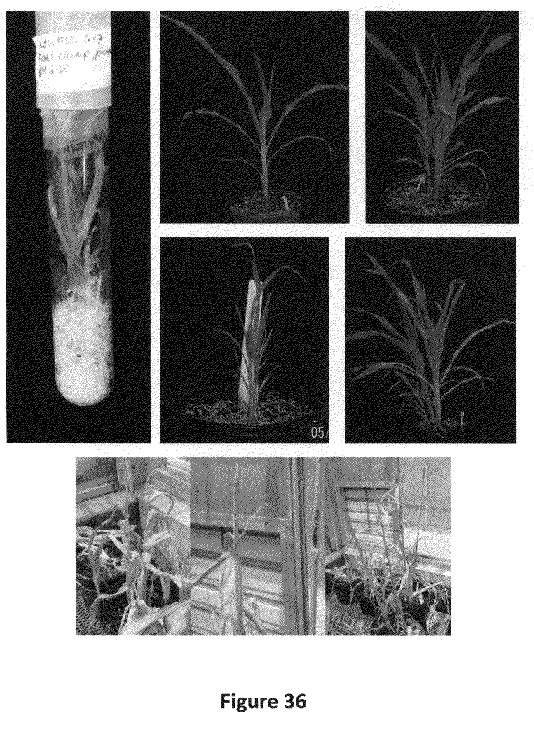

More than 180 plates of maize callus were bombarded and five resistant clones were obtained. Of these, 10 plants were regenerated but only four survived to maturity. These plants were weak and proved to be infertile (FIG. 36).

### 3.2 Molecular and Enzyme Assays for XYL on Maize and Tobacco

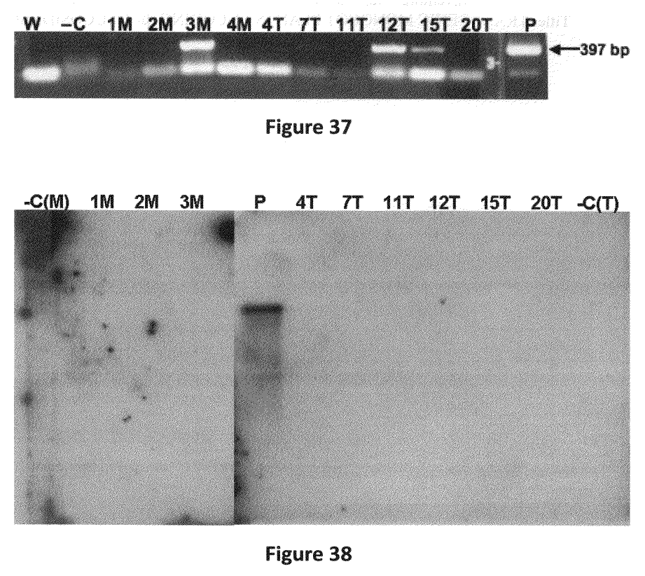

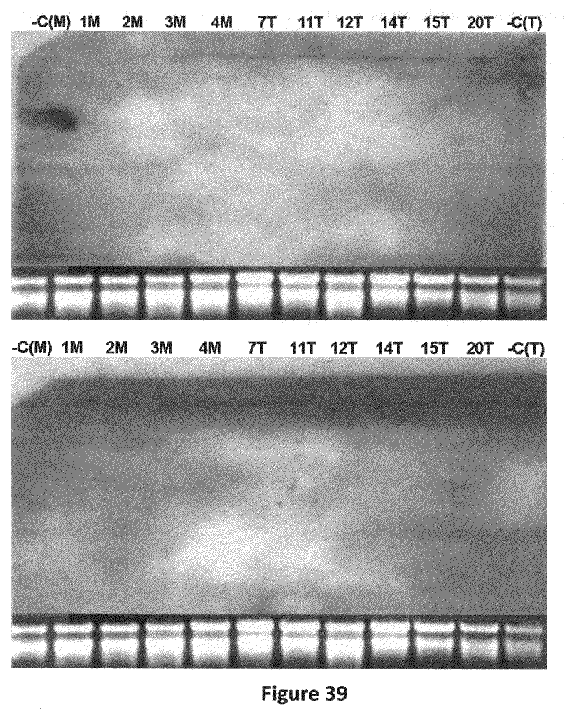

Molecular analyses on maize and tobacco plants were performed. PCR (FIG. 37) revealed that one of the maize plants and two of the tobacco plants were likely to contain the XYL1 gene sequence. All lanes, including the water, negative (untransformed control) and positive control (XYL1 plasmid) had primer dimers (appearing as a band of smaller molecular weight), indicating that primer concentration was likely too high.

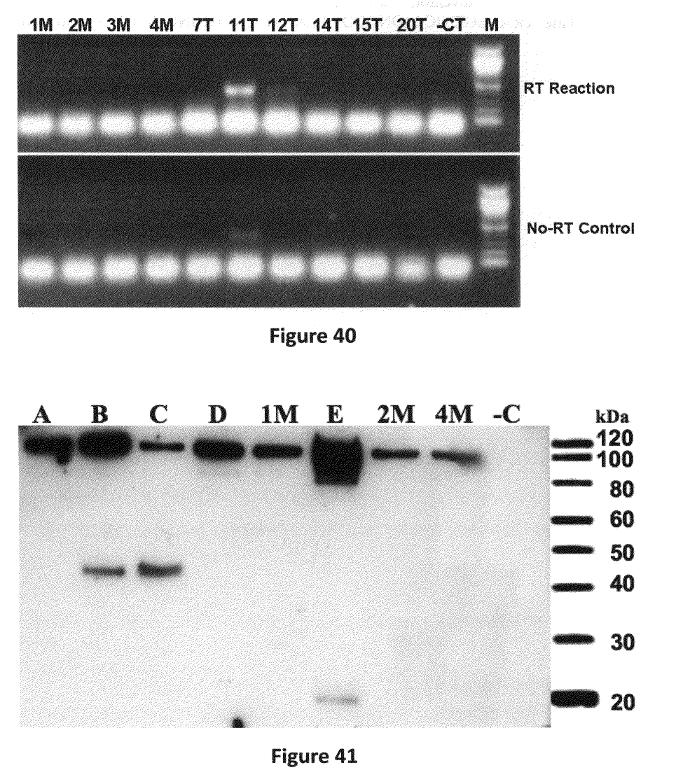

DNA from maize and tobacco plants did not show any hybridization with the XYL1 probe in a Southern blot (FIG. 38) although plants 3M, 12T and 15T showed an amplified band after PCR. Because the method used for hybridization and detection was non-radioactive (DIG), it may not have been sensitive enough to detect a low copy number in transgenic plants. Similarly, northern blots for maize and tobacco plants did not show any hybridization (FIG. 38). RT-PCR (FIG. 40) showed two plants had a band (11T and 12T); however, the band for 11T was also present in the no-RT control, indicating that the RNA was contaminated with DNA, and this is not a true amplification from RNA. It was possible the band seen in 12T was legitimate, although it is very faint. All samples had large primer dimers, indicating lack of template or too much primer in the reaction. This means that it is unlikely that any of the plants were producing XYL RNA.

A Western blot was performed on eight of 10 maize plants (two being left out because of lack of sufficient protein) (FIG. 41). Tissue had been collected prior to plant death. In two of the plants, 2 and 3, a single band with a molecular weight of around 40 kDa was detected and in plant 6, a band of around 20 kDa was detected. All of the transgenic plants had a large, 100-kDa protein that was absent in the non-transformed control.

The assay for reducing sugars using PABAH (Lever 1972) was used on the plants. Absorbance readings at 410 nm for the plants after subtracting the 0 time point is shown in for maize in Table 9 and tobacco (both total protein concentrate and extracellular fluid wash; Herbers et al. 1995) in Table 10. An increase of 0.2 to 1 shows activity. None of the plants showed any significant activity that was greater than negative control.

TABLE 9
Maize plants tested for XYL activity using PABAH assay for reducing
sugars. Absorbance taken at 410 nm for 0 min, 30 min and 4 h are
shown, along with the adjusted values (0 min subtracted).

| Plant | 0 min | 30 min | 30 min adj. | 4 h | 4 h adj. |
|---|---|---|---|---|---|
| A | 1.3362 | 0.9634 | −0.3728 | 0.9630 | −0.3732 |
| B | 1.4841 | 1.0378 | −0.4463 | 1.0775 | −0.4066 |
| C | 0.1981 | 0.2726 | 0.0745 | 0.3803 | 0.1822 |
| D | 0.5352 | 0.4622 | −0.073 | 0.4419 | −0.0933 |
| E | 0.3742 | 0.3270 | −0.0472 | 0.3384 | −0.0358 |
| F | 0.2018 | 0.2131 | 0.0113 | 0.2642 | 0.0624 |
| 1M | 0.3294 | 0.3254 | −0.004 | 0.3356 | 0.0062 |
| 2M | 0.7939 | 0.6459 | −0.148 | 0.6316 | −0.1623 |
| 3M | 0.2766 | 0.2637 | −0.0129 | 0.2803 | 0.0037 |
| 4M | 0.3503 | 0.3355 | −0.0148 | 0.3735 | 0.0232 |
| -C | 0.5277 | 0.4556 | −0.0721 | 0.5110 | −0.0167 |

TABLE 10
Tobacco plants total protein concentrate and
extracellular fluid wash tested for XYL activity using
PABAH assay for reducing sugars. Absorbance taken at
410 nm for 0 min, 30 min and overnight are shown, along
with the adjusted values (0 min subtracted).

| | | | | | |
|---|---|---|---|---|---|
| | | | 30 min | Over | O/n |
| Plant | 0 min | 30 min | adj. | night | adj. |

Total protein concentrate

| | | | | | |
|---|---|---|---|---|---|
| -C | 0.481 | 0.4839 | 0.0029 | 0.5454 | 0.0644 |
| 4 | 0.3341 | 0.3968 | 0.0627 | 0.4799 | 0.1458 |
| 7 | 0.2374 | 0.3268 | 0.0894 | 0.2954 | 0.058 |
| 11 | 0.2729 | 0.326 | 0.0531 | 0.3191 | 0.0462 |
| 12 | 0.3426 | 0.3818 | 0.0392 | 0.4336 | 0.091 |
| 14 | 0.3875 | 0.4161 | 0.0286 | 0.5045 | 0.117 |
| 15 | 0.2866 | 0.3193 | 0.0327 | 0.3801 | 0.0935 |
| 20 | 0.3204 | 0.3247 | 0.0043 | 0.3954 | 0.075 |

Extracellular fluid wash

| | | | | | |
|---|---|---|---|---|---|
| -C | 0.213 | 0.2597 | 0.0467 | 0.2139 | 0.0009 |
| 4 | 0.2843 | 0.2957 | 0.0114 | 0.2497 | −0.0346 |
| 7 | 0.2465 | 0.2875 | 0.041 | 0.218 | −0.0285 |
| 11 | 0.2317 | 0.2587 | 0.027 | 0.1709 | −0.0608 |
| 12 | 0.2619 | 0.291 | 0.0291 | 0.2394 | −0.0225 |
| 14 | 0.254 | 0.2746 | 0.0206 | 0.2114 | −0.0426 |
| 15 | 0.2625 | 0.2903 | 0.0278 | 0.2069 | −0.0556 |
| 20 | 0.2603 | 0.2841 | 0.0238 | 0.2224 | −0.0379 |

After all the analyses were performed, it was discovered that the company that had made the construct, Norclone, had used available restriction sites in the original plasmid for cloning the DNA fragment into the vector (FIGS. 33 and 34). Unfortunately, these sites cut a fragment that contained part of the plasmid backbone (i.e., “junk”). The gene remained in frame relative to the rest of the construct and no stop codons were present. In addition, this DNA sequence contained the native fungal secretory signal peptide. This shows the sensitivity of the restriction sites.

Several attempts were made to correct the construct by removing both the junk and the fungal signal peptide, but failed due to mutations or incorrect orientation. Because neither construct (with and without the fungal signal peptide) had worked previously in tobacco (Apel, 1996), it was unlikely that it could work in maize even with a different signal peptide (i.e., apoplast). As a result, a new project with a new xylanase (XYL from A. cellulolyticus, gift from Edenspace Corp., Manhattan, Kans.) was begun.

### 3.3 Cloning of New XYL

3.3.1 pMZ766 Vector

Primers were ordered that added SacI sticky ends to the XYL gene, which were then used in PCR and the gene was amplified. As before, the PCR fragment was cloned into a T-vector, sequenced, and then cloned into the pMZ766 backbone. Several attempts were made but all of the resulting clones had the gene in the reverse orientation. So this project was given to another researcher. Several tobacco plants resistant to kanamycin were generated but PCR revealed that none of them out of the 11 that were tested had the XYL gene.

### 3.3.2 ImpactVectors

An additional set of primers was ordered that added NcoI and BglII sticky ends to the XYL gene. This fragment was to be cloned into the five ImpactVectors. These vectors are specifically for plant transformation and employ the rubisco small subunit (RbcS1) promoter from the Asteraceous chrysanthemum and 1 kb of the RbcS1 terminator sequence. Each one utilizes a different targeting sequence: cytoplasm, secretory pathway, endoplasmic reticulum, chloroplast and mitochondria. In addition, the vectors have a cmyc-tag allowing identification of expressed proteins using commercially available monoclonal antibodies and a six histidine His-tag for protein purification using a nickel column.

After cloning was accomplished and verified with sequencing, each of the five targeting constructs was cloned into the binary vector provided with the ImpactVectors, pBINPLUS. As before, the sequences were verified after cloning procedures. In addition, the vector was used to transform Agrobacterium. After the cloning was accomplished, the plasmid DNA in the simple vector was prepared for bombardment by maxiprep. Maize callus transformation using the ImpactVectors with XYL is currently underway.

### Appendix C. CBH1 and FLC in Maize

### 1. Introduction

Cellobiohydrolase I from T. reesei is an exoglucanase previously shown to have activity when expressed in tobacco (Dai et al. 1999). In this set of experiments, it was attempted to do the same in maize.

### 2. Materials and Methods

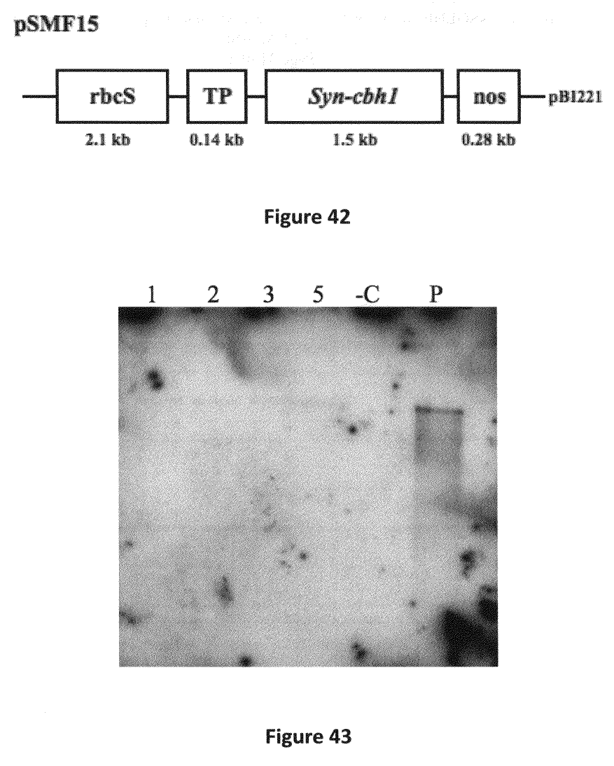

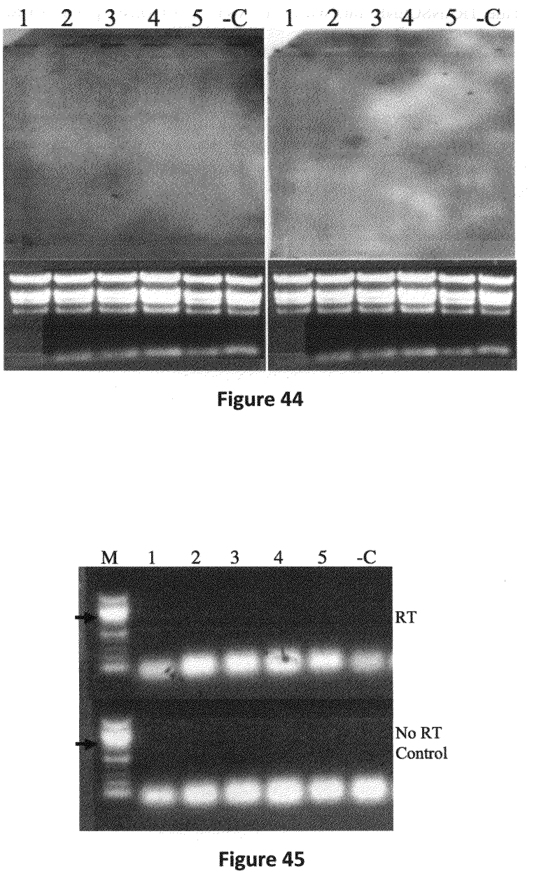

A construct containing a synthetic CBH1 that had been codon modified for use in tobacco transformation was made previously in the Sticklen laboratory. It had the rice Rubisco (rbcS) small subunit promoter, the rbcS chloroplast signal peptide also from rice, and the Agrobacterium nopaline synthase (nos) 3′ non-coding region (FIG. 42).

Maize callus production, bombardment, selection and regeneration were carried out as described previously in Materials and Methods (page 50), as were DNA, RNA and protein extraction (pages 51, 53 and 54) and Southern, northern and western blotting (pages 53 and 57). For Southern blots, 15 μg genomic DNA was digested overnight with HindIII and fractionated on a 1% agarose gel. PCR and RT-PCR were performed using the following primers and conditions: Syn-cbh1-F: 5′ TCT TGA TGG TGC TGC TTA CG 3′ (SEQ ID NO: 11) and Syn-cbh1-R: 5′ CCA AAC TCA GCT TCC TCA GC 3′ (SEQ ID NO: 12) (801 bp); 94° C. for 3 min; 35 cycles of 94° C. for 45 s, 56° C. for 45 s, 72° C. for 45 s; 72° C. for 10 min. The probe used for Southern and northern hybridization and detection was the 801-bp fragment generated by PCR described above. For sequencing, an additional primer was ordered, Syn-cbh1-R-Seq: 5′CGT AAG CAG CAC CAT CAA GA 3′(SEQ ID NO: 13).

### 3. Results and Discussion

3.1 Transformation of Maize with Syn-CBH1 and FLC

Seventy-five plates of maize callus were bombarded with a combination of pMSF15 (containing Syn-CBH1) and pGreen (containing FLC) and 15 resistant clones were obtained. From these, 12 plants were regenerated but only five survived to maturity.

### 3.2 Molecular Analyses for Syn-CBH1 on Maize

Molecular analyses on maize plants were performed. DNA from maize plants showed a very faint hybridization with the Syn-CBH1 probe in a Southern blot that was not present in non-transgenic control, all around the same size as the plasmid band (FIG. 43).

However, northern blots did not show any hybridization and RT-PCR showed that none of the plants had a band. All samples had large primer dimers, indicating lack of template or too much primer in the reaction. This means that it is unlikely that any of the plants were producing Syn-CBH1 RNA.

After all the analyses were performed, when it was discovered that some of our constructs had mistakes, we decided to check all the constructs, so pMSF15 was sequenced. Sequencing revealed many frameshifts caused by deletions, a large fragment of the CaMV35S promoter, numerous stop codons, and a deletion in the start codon. It was discovered that major mistakes had been made during the cloning.

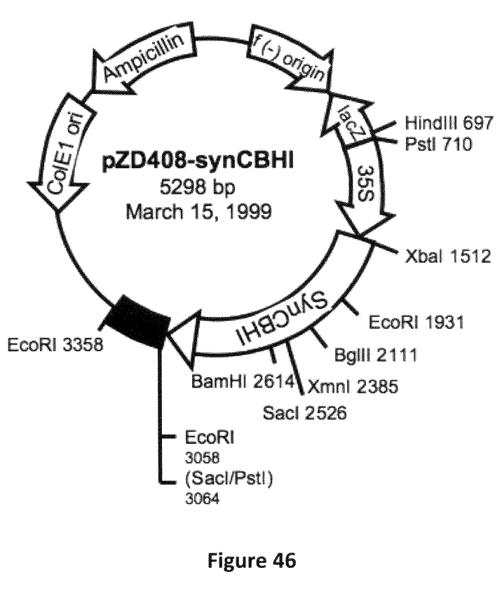

To fix the construct, new primers were ordered to amplify the rbcS transit peptide with XbaI sticky ends and insert it between the 35S promoter and Syn-CBH1 gene in the original Syn-CBH1 plasmid, pZD408 (FIG. 46).

This was done, and the sequence checked in the T-vector. Both pZD408 and the T-vector were digested with XbaI and the correct fragments ligated together. PCR was performed to verify orientation. The construct was then sequenced. A single point deletion in the middle of the Syn-CBH1 gene was discovered; this was seen in more than one clone. So, pZD408 was sent to sequencing, and it was found that the mutation was present in the original vector's gene sequence. Another plasmid that had been used in the construction of pZD408 in Dr. Dai's laboratory contained the correct sequence (pZD394).

However, this plasmid did not have any promoter or terminator sequences, just the Syn-CBH1 gene in a lacZ multiple cloning site. Originally, the plan was to cut out the correct sequence and excise the incorrect sequence and re-ligate them, but there proved to be no available restriction sites. Correcting the construct then became a complicated matter of amplifying the various pieces adding sticky ends and ligating them together sequentially in a simple vector. Eventually, the project was abandoned due to its complexity and lack of a suitable vector.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

### REFERENCES

Andersson-Gunneras, S., E. J. Mellerowicz, J. Love, B. Segerman, Y. Ohmiya, P. M. Coutinho, P. Nilsson, B. Henrssat, T. Moritz and B. Sundberg. 2006. Biosynthesis of cellulose-enriched tension wood in Populus: global analysis of transcripts and metabolites identifies biochemical and developmental regulators in secondary wall biosynthesis. The Plant J. 45(2): 144-165.
Apel, P. C. 1996. The Cloning, Comparison, and Expression of Three Family G Endo B-1,4-xylanase Genes of the Maize Fungal Pathogen Cochliobolus Carbonum and Analysis of Their Importance For Pathogenicity on Maize. Michigan State University.
Arioli, T., L. Peng, A. S. Betzner, J. Burn, W. Wittke, W. Herth, C. Camilleri, H. Höfte, J. Plazinski, R. Birch, A. Cork, J. Glover, J. Redmond, R. E. Williamson. 1998. Molecular Analysis of Cellulose Biosynthesis in Arabidopsis. Science 279(5351): 717-720.
Armstrong, C. L., and C. E. Green. 1985. Establishment and maintenance of friable, embryogenic maize callus and the involvement of L-proline. Planta 164:207-214.
Armstrong, C. L., C. E. Green, and R. L. Phillips. 1991. Development and availability of germplasm with high Type II culture formation response. Maize Genet. Coop. Newslett 65:92-93.
Baker, J. O., W. S. Adney, R. A. Nieves, S. R. Thomas, D. B. Wilson, and M. E. Himmel. 1994. A new thermostable endoglucanase, Acidothermus cellulolyticus E1. Synergism with Trichoderma reesei CBH I and comparison to Thermomonospora fusca E5. Appl. Biochem. Biotech. 45/46:245-256.
Baucher, M., M. A. Bernard-vailhé, B. Chabbert, J. M. Besle, C. Opsomer, M. Van Montagu, and J. Botterman. 1999. Down-regulation of cinnamyl alcohol dehydrogenase in transgenic alfalfa (Medicago sativa L.) and the effect on lignin composition and digestibility. Plant Mol. Biol. 39(3): 437-447.
Biomass Program Net Energy Balance for Bioethanol Production and Use. http://www1.eere.energy.gov/biomass/net_energy_bala nce.html (accessed Aug. 1, 2007).
Biswas, G. C. G., C. Ransom, and M. Sticklen. 2006. Expression of biologically active Acidothermus cellulolyticus endoglucanase in transgenic maize plants. Plant Science 171(5): 617-623.
Blaschke, L., M. Legrand, C. Mai, and A. Polle. 2004. Lignification and structural biomass production in tobacco with suppressed caffeic/5-hydroxy ferulic acid-O-methyl transferase activity under ambient and elevated CO₂ concentrations. Physiol. Plant. 121(1): 75-83.
Bohmert, K., I. Balbo, A. Steinbuchel, G. Tischendorf, and L. Willmitzer. 2002. Constitutive Expression of the β-Ketothiolase Gene in Transgenic Plants. A Major Obstacle for Obtaining Polyhydroxybutyrate-Producing Plants. Plant Physiol. 128(4): 1282-1290.
Boisset, C., C. Pétrequin, H. Chanzy, B. Henrissat, and M. Schülein. 2001. Optimized mixtures of recombinant Humicola insolens cellulases for the biodegradation of crystalline cellulose. Biotechnology and bioengineering 72(3): 339-45.
Bolwell, G. P. 2000. Biosynthesis of Plant Cell Wall Polysaccharides. Trends Glycosci. Glycotech. 12(65): 143-160.
Boominathan, K., and C. A. Reddy. 1992. Fungal degradation of lignin: biotechnological applications. In Fungal Biotechnology, 763-882.
Bordetsky, A., R. Hwang, A. Korin, D. Lovaas, and L. Tonachel. 2005. SECURING AMERICA: Solving Our Oil Dependence Through Innovation. Natural Resources Defense Council.
Bothast, R. J., and M. A. Schlicher. 2005. Biotechnological processes for conversion of corn into ethanol. Appl. Microbiol. Biotech. 67(1): 19-25.
Boudet, A. M., S. Kajita, J. Grima-Pettenati, and D. Goffner. 2003. Lignins and lignocellulosics: a better control of synthesis for new and improved uses. Trends Plant Sci. 8(12): 576-581.
Boudet, Alain-M. 2000. Lignins and lignification: Selected issues. Plant Physiol. Biochem. 38(1-2): 81-96.
Bradford, M. 1976. A rapid and sensitive method for quantitation of microgram quantities of protein utilizing the principle of protein-dye binding. Anal. Biochem. 72:248-254.
Bruins, M. E., A. E. M. Janssen, and R. M. Boom. 2001. Thermozymes and their applications-A review of recent literature and patents. Appl. Biochem. Biotech. 90:155-186.
Cai, Y. J., S. J. Chapman, J. A. Buswell, and S. T. Chang. 1999. Production and Distribution of Endoglucanase, Cellobiohydrolase, and beta-Glucosidase Components of the Cellulolytic System of Volvariella volvacea, the Edible Straw Mushroom. Appl. Environ. Microbiol. 65(2): 553-559.
Cao, J., X. Duan, D. McElroy, and R. Wu. 1992. Regeneration of herbicide resistant transgenic rice plants following microprojectile-mediated transformation of suspension culture cells. Plant Cell Reports 11:586-591.
Carpita, N. C., and D. M. Gibeaut. 1993. Structural models of primary cell walls in flowering plants: consistency of molecular structure with the physical properties of the walls during growth. The Plant J. 3(1): 1-30.
Carpita, N. and M. McCann. 2002. Chapter 2. The cell wall. In Biochemistry & Molecular Biology of Plants, ed. B. Buchanan, W. Gruissem, and R. L. Jones, 52-108. John Wiley & Sons.
Carräo-Panizzi, M. C., and J. R. Bordingnon. 2000. Activity of beta-glucosidase and levels of isoflavone glucosides in soybean cultivars affected by the environment. Pesquisa Agropecuária Brasileira 35:873-878.
Chabannes, M., A. Barakate, C. Lapierre, J. M. Marita, J. Ralph, M. Pean, S. Danoun, C. Halpin, J. Grima-Pettenati and A. M. Boudet. 2001. Strong decrease in lignin content without significant alteration of plant development is induced by simultaneous down-regulation of cinnamoyl CoA reductase (CCR) and cinnamyl alcohol dehydrogenase (CAD) in tobacco plants. The Plant J. 28(3): 257-270.
Chiang, C. M., F. S. Yeh, L. F. Huang, T. H. Tseng, M. C. Chung, C. S. Wang, H. S. Lur, J. F. Shaw, and S. M. Yu. 2005. Expression of a bi-functional and thermostable amylopullulanase in transgenic rice seeds leads to autohydrolysis and altered composition of starch. Mol. Breeding. 15:125-143.
Chu, C. C., C. C. Wang, C. S. Sun, C. Hus, K. C. Yin, C. Y. Chu, et al. 1975. Establishment of an efficient medium for another culture of rice through comparative experiments on the nitrogen source. Scientia Sinica 18:659-668.
Conrad, U. and U. Fiedler. 1998. Compartment-specific accumulation of recombinant immunoglobulins in plant cells: an essential tool for antibody production and immunomodulation of physiological functions and pathogen activity. Plant Mol. Biol. 38(1): 101-109.
D'Souza, T. M., C. S. Merritt, and C. A. Reddy. 1999. Lignin-Modifying Enzymes of the White Rot Basidiomycete Ganoderma lucidum. Appl. Environ. Microbiol. 65(12): 5307-5313.
Dai, Z., B. S. Hooker, R. D. Quesenberry, and J. Gao. 1999. Expression of Trichoderma reesei exo-cellobiohydrolase I in transgenic tobacco leaves and calli. Appl. Biochem. Biotech. 77-79:689-99.
Dai, Z., B. S. Hooker, D. B. Anderson, and S. R. Thomas. 2000a. Expression of Acidothermus cellulolyticus endoglucanase E1 in transgenic tobacco: biochemical characteristics and physiological effects. Trans. Res. 9, no. 1 (February 1): 43-54.
______. 2000b. Improved plant-based production of E1 endoglucanase using potato: expression optimization and tissue targeting. Mol. Breeding. 6:277-285.
Dai, Z., B. S. Hooker, R. D. Quesenberry, and S. R. Thomas. 2005. Optimization of Acidothermus cellulolyticus Endoglucanase (E1) Production in Transgenic Tobacco Plants by Transcriptional, Post-transcription and Post-translational Modification. Trans. Res. 14(5): 627-643.
Dean, J. F. D. 2004. Synthesis of Lignin in Transgenic and Mutant Plants. In Biotechnology of Biopolymers: From Synthesis to Patents, ed. A. Steinbuchel and Y. Doi, 4-21. Wiley-VCH.
Decker, S. R., W. S. Adney, E. Jennings, T. B. Vinzant, and M. E. Himmel. 2003. Automated Filter Paper Assay for Determination of Cellulase Activity. Appl. Biochem. Biotechnol. 107:689-704.
Demain, A. L., M. Newcomb, and J. H. D. Wu. 2005. Cellulase, Clostridia, and Ethanol. Microbiol. Mol. Biol. Rev. 69(1): 124-154.
Ding, S. Y., and M. E. Himmel. 2006. The Maize Primary Cell Wall Microfibril: A New Model Derived from Direct Visualization. J. Agric. Food Chem. 54(3): 597-606.
Dodd, A. N., N. Salathia, A. Hall, E. Kevei, R. Toth, F. Nagy, J. M. Hibberd, A. J. Millar, and A. A. R. Webb. 2005. Plant Circadian Clocks Increase Photosynthesis, Growth, Survival, and Competitive Advantage. Science 309(5734): 630-633.
Eggeman, T., and R. T. Elander. 2005. Process and economic analysis of pretreatment technologies. Biores. Tech. 96(18): 2019-2025.
Eriksson, M. E., M. Israelsson, O. Olsson, and T. Moritz. 2000. Increased gibberellin biosynthesis in transgenic trees promotes growth, biomass production and xylem fiber length. Nat. Biotech. 18, no. 7 (July): 784-788.
Fischer, R., E. Stoger, S. Schillberg, P. Christou, and R. M. Twyman. 2004. Plant-based production of biopharmaceuticals. Curr. Opin. Plant Biol. 7(2): 152-158.
Genencor Celebrates Major Progress in the Conversion of Biomass to Ethanol—Genencor a Danisco division. http://www.genencor.com/cms/connect/genencor/media_relations/news/archive/2004/gen_{—}211004_en.htm (accessed Aug. 3, 2007).
Ghose, T. K. 1987. Measurement of cellulase activities. Pure Appl. Chem. 59(2): 257-268.
Greene, N., F. E. Celik, B. Dale, M. Jackson, K. Jayawardhana, H. Jin, E. D. Larson, M. Laser, L. Lynd, D. MacKenzie, J. Mark, J. McBride, S. McLaughlin and D. Saccardi. 2004. Growing Energy: How Biofuels Can Help End America's Oil Dependence.
Haigler, C. H. 2006. Establishing the cellular and biophysical context of cellulose synthesis. In, The Science and Lore of the Plant Cell Wall: Biosynthesis, Structure and Function. ed. T. Hayashi, 368. Brown Walker Press (FL).
Hatakka, A. 1994. Lignin-modifying enzymes from selected white-rot fungi: production and role in lignin degradation. FEMS Microbiol. Rev. 13:125-135.
Hellens, R., P. Mullineaux, and H. Klee. 2000. Technical Focus: A guide to Agrobacterium binary Ti vectors. Trends Plant Sci. 5(10): 446-451.
Herbers, K., I. Wilke, and U. Sonnewald. 1995. A Thermostable Xylanase from Clostridium thermocellum Expressed at High Levels in the Apoplast of Transgenic Tobacco Has No Detrimental Effects and Is Easily Purified. Nat. Biotech. 13(1): 63-66.
Hong, C. Y., K. J. Cheng, T. H. Tseng, C. S. Wang, L. F. Liu, and S. M. Yu. 2004. Production of two Highly Active Bacterial Phytases with Broad pH Optima in Germinated Transgenic Rice Seeds. Trans. Res. 13(1): 29-39.
Horn, M. E., S. L. Woodard, and J. A. Howard. 2004. Plant molecular farming: systems and products. Plant Cell Rep. 22(10): 711-720.
Hoshino, E., M. Shiroishi, Y. Amano, and T. Kanda. 1997. Synergistic Actions of Exo-Type Cellulases in the Hydrolysis of Cellulose with Different Crystallinities. J. Fermentation Bioeng. 84(4): 300-306.
Houghton, J., J. Ferrel, and S. Weatherwax. 2005. From Biomass to Biofuels: A Roadmap to the Energy Future. Proceedings of the Biomass to Biofuels Workshop, December 7.
Howard, J. A., and E. Hood. 2005. Bioindustrial and Biopharmaceutical Products Produced in Plants. Adv. Agron. (85)91-124, Elsevier Inc.
Howard, R. L., E. Abotsi, E. L. Jansen van Rensburg, and S. Howard. 2003. Lignocellulose biotechnology: issues of bioconversion and enzyme production. Afr. J. Biotech. 2(12): 602-619.
Hu, W. J., S. A. Harding, J. Lung, J. L. Popko, J. Ralph, D. D. Stokke, C. J. Tsai, and V. L. Chiang. 1999. Repression of lignin biosynthesis promotes cellulose accumulation and growth in transgenic trees. Nat. Biotech. 17(8): 808-812.
Hyunjong, B., D. S. Lee, and I. Hwang. 2006. Dual targeting of xylanase to chloroplasts and peroxisomes as a means to increase protein accumulation in plant cells. J. Exp. Bot. 57(1): 161-169.
Ingledew, W. M. 1995. The Biochemistry of Ethanol Production. In The Alcohol Textbook, ed. T. P. Lyons, D. Kelsall, and J. Murtagh, 55-79. Nottingham, UK: Nottingham University Press.
Kabel, M. A., M. J. E. C. van der Maarel, G. Klip, A. G. J. Voragen, and H. A. Schols. 2006. Standard assays do not predict the efficiency of commercial cellulase preparations towards plant materials. Biotech. Bioeng. 93(1): 56-63.
Kawagoe, Y., and D. P. Delmer. 1997. Pathways and genes involved in cellulose biosynthesis. Gen. Eng. 19:63-87.
Kim, S., and B. E. Dale. 2004. Global potential bioethanol production from wasted crops and crop residues. Biomass Bioen. 26(4): 361-375.
Kimura, T., T. Mizutani, K. Sakka, and K. Ohmiya. 2003a. Stable expression of a thermostable xylanase of Clostridium thermocellum in cultured tobacco cells. J. Biosci. Bioeng. 95(4): 397-400.
Kimura T., T. Mizutani, T. Tanaka, T. Koyama, K. Sakka, and K. Ohmiya. 2003b. Molecular breeding of transgenic rice expressing a xylanase domain of the xynA gene from Clostridium thermocellum. Appl. Microbiol. Biotechnol. 62(4): 374-379.
Kiran, N. S., L. Polanská, R. Fohlerova, P. Mazura, M. Válková, M. Smeral, J. Zouhar, J. Malbeck, P. I. Dobrev, I. Machácková, and B. Brzobohaty. 2006. Ectopic over-expression of the maize beta-glucosidase Zm-p60.1 perturbs cytokinin homeostasis in transgenic tobacco. J. Exp. Bot. 57(4): 985-96.
Kirk, T. K., and R. L. Farrell. 1987. Enzymatic “combustion”: the microbial degradation of lignin. Annu. Rev. Microbiol. 41:465-505.
Knauf, M., and M. Moniruzzaman. 2004. Lignocellulosic biomass processing: A perspective. Int. Sugar J. 106:147-150.
Lever, M. 1972. A new reaction for colorimetric determination of carbohydrates. Anal. Biochem. 47(1): 273-279.
Li, L., Y. Zhou, X. Cheng, J. Sun, J. M. Marita, J. Ralph, and V. L. Chiang. 2003. Combinatorial modification of multiple lignin traits in trees through multigene cotransformation. Proc. Nat. Acad. Sci. USA. 100(8): 4939-4944.
Liu, H. L., W. S. Li, T. Lei, J. Zheng, Z. Zhang, X. F. Yan, Z. Z. Wang, Y. L. Wang and L. S. Si. 2005. Expression of Human Papillomavirus type 16 L1 protein in transgenic tobacco plants. Act. Biochim. Biophys. Sin. 37:153-158.
Luo, Y., J. L. Chen, J. F. Reynolds, C. B. Field, and H. A. Mooney. 1997. Disproportional increases in photosynthesis and plant biomass in a Californian grassland exposed to elevated CO₂: a simulation analysis. Funct. Ecol. 11(6): 696-704.
Lynd, L. R., W. H. van Zyl, J. E. McBride, and M. Laser. 2005. Consolidated bioprocessing of cellulosic biomass: an update. Curr. Opin. Biotech. 16:577-583.
Michaels, S., and R. Amasino. 1999. FLOWERING LOCUS C Encodes a Novel MADS Domain Protein That Acts as a Repressor of Flowering. The Plant Cell 11:949-956.
Miller, G. L. 1959. Use of Dinitrosalicylic Acid Reagent for Determination of Reducing Sugar. Anal. Chem. 31(3): 426-428.
Mosier, N., C. Wyman, B. Dale, R. Elander, Y. Y. Lee, M. Holtzapple, and M. Ladisch. 2005. Features of promising technologies for pretreatment of lignocellulosic biomass. Biores. Tech. 96(6): 673-686.
Murray C., P. W. Sutherland, M. M. Phung, M. T. Lester, R. K. Marshall and T. Christeller. 2002. Expression of Biotin-Binding Proteins, Avidin and Streptavidin, in Plant Tissues Using Plant Vacuolar Targeting Sequences. Trans. Res. 11(2): 199-214.
Nuttall, J., N. Vine, J. L. Hadlington, P. Drake, L. Frigerio, and J. K. C. Ma. 2002. ER-resident chaperone interactions with recombinant antibodies in transgenic plants. Eur. J. Biochem. 269(24): 6042-6051.
O'Neill, M. A., T. Ishii, P. Albersheim, and A. G. Darvill. 2004. Rhamnogalacturonan II: structure and function of a borate cross-linked cell wall pectic polysaccharide. Annu. Rev. Plant Biol. 55:109-39.
Oraby, H., B. Venkatesh, B. Dale, R. Ahmad, C. Ransom, J. Oehmke, and M. Sticklen. 2007. Enhanced conversion of plant biomass into glucose using transgenic rice-produced endoglucanase for cellulosic ethanol. Trans. Res. Published Online First: 20 Jan. 2007.
Pan, X., N. Gilkes, J. Kalda, K. Pye, S. Saka, D. Gregg, K. Ehara, D. Xie, D. Lam and J. Saddler. 2005. Biorefining of softwoods using ethanol organosolv pulping: Preliminary evaluation of process streams for manufacture of fuel-grade ethanol and co-products. Biotech. Bioeng. 90(4): 473-481.
Patel M., J. S. Johnson, R. I. S. Brettell, J. Jacobsen, and G. P. Xue. 2000. Transgenic barley expressing a fungal xylanase gene in the endosperm of the developing grains. Mol. Breed. 6(1): 113-124.
Perlack, R. D., L. L. Wright, A. F. Turhollow, R. L. Graham, B. J. Stokes, and D. C. Erbach. 2005. Biomass as a Feedstock for a Bioenergy and Bioproducts Industry: The Technical Feasibility of a Billion-Ton Annual Supply. U.S. Dept. of Energy and U.S. Dept. of Agriculture.
Persson, S., H. Wei, J. Milne, G. P. Page, and C. R. Somerville. 2005. Identification of genes required for cellulose synthesis by regression analysis of public microarray data sets. Proc. Nat. Acad. Sci. USA. 102(24): 8633-8638.
Pilate, G., E. Guiney, K. Holt, M. Petit-Conil, C. Lapierre, J. C. Leple, B. Pollet, I. Mila, E. A. Webster, H. G. Marsttrop, D. W. Hopkins, L. Jouanin, W. Boerjan, W. Schuch, D. Cornu, and C. Halpin. 2002. Field and pulping performances of transgenic trees with altered lignification. Nat. Biotech. 20(6): 607-612.
Qi, B., T. Fraser, S. Mugford, G. Dobson, O, Sayanova, J. Butler, J. A. Napier, A. K. Stobart, and C. M. Lazarus. 2004. Production of very long chain polyunsaturated omega-3 and omega-6 fatty acids in plants. Nat. Biotech. 22(6): 739-745.
Ragauskas, A. J., C. K. Williams, B. H. Davison, G. Britovsek, J. Cairney, C. A. Eckert, W. J. Frederick, J. P. Hallett, D. J. Leak, C. L. Liotta, J. R. Mielenz, R. Murphy, R. Templer, and T. Tschaplinski. 2006. The Path Forward for Biofuels and Biomaterials. Science 311(5760): 484-489.
Ralph, J. 2006. What makes a good monoligol substitute? In The Science And Lore of the Plant Cell Wall: Biosynthesis, Structure And Function, ed. T. Hayashi, 367. Brown Walker Press (FL).
Ralph, J., T. Akiyama, H. Kim, F. Lu, P. F. Schatz, J. M. Marita, S. A. Ralph, M. S. S. Reddy, F. Chen, and R. A. Dixon. 2006. Effects of Coumarate 3-Hydroxylase Down-regulation on Lignin Structure. J. Biol. Chem. 281(13): 8843-8853.
Ransom, C. B. 2007. Production and Analysis of Biologically-Active Cellulases for Ethanol Fuel in Maize Biomass. Michigan State University.
Ransom, C. B., V. Balan, G. C. G. Biswas, B. E. Dale, E. Crockett, and M. B. Sticklen. 2007. Heterologous Acidothermus cellulolyticus 1,4-β-Endoglucanase E1 Produced Within the Corn Biomass Converts Corn Stover Into Glucose. Appl. Biochem. Biotech. 36-140:207-220.
Reddy, M. S. S., F. Chen, G. Shadle, L. Jackson, H. Aljoe, and R. A. Dixon. 2005. Targeted down-regulation of cytochrome P450 enzymes for forage quality improvement in alfalfa (Medicago sativa L.). Proc. Nat. Acad. Sci. USA. 102(46): 16573-16578.
Reggi S., S. Marchetti, T. Patti, F. De Amicis, R. Cariati, B. Bembi, and C. Fogher. 2005. Recombinant human acid β-glucosidase stored in tobacco seed is stable, active and taken up by human fibroblasts. Plant Mol. Biol. 57(1): 101-113.
Renewable Fuels Association. 2007. Building New Horizons: Ethanol Industry Outlook 2007.
RFA—The Industry—Plant Locations. http://www.ethanolrfa.org/industry/locations/(accessed Aug. 5, 2007).
Richards, R. A. 2000. Selectable traits to increase crop photosynthesis and yield of grain crops. J. Exp. Bot. 51, no. suppl_{—}1: 447-458.
Ridley, B. L., M. A. O'Neill, and D. Mohnen. 2001. Pectins: structure, biosynthesis, and oligogalacturonide-related signaling. Phytochem. 57(6): 929-967.
Saghai-Maroof, M. A., K. M. Soliman, R. A. Jorgensen, and R. W. Allard. 1984. Ribosomal DNA Spacer-Length Polymorphisms in Barley: Mendelian Inheritance, Chromosomal Location, and Population Dynamics. PNAS 81, no. 24: 8014-8018.
Sahrawy, M., C. Avila, A. Chueca, F. M. Canovas, and J. Lopez-Gorge. 2004. Increased sucrose level and altered nitrogen metabolism in Arabidopsis thaliana transgenic plants expressing antisense chloroplastic fructose-1,6-bisphosphatase. J. Exp. Bot. 55(408): 2495-2503.
Salehi, H., C. B. Ransom, H. F. Oraby, Z. Seddighi, and M. B. Sticklen. 2005. Delay in flowering and increase in biomass of transgenic tobacco expressing the Arabidopsis floral repressor gene FLOWERING LOCUS C. J. Plant Phys. 162, no. 6: 711-717.
Sambrook, J., E. F. Fritsch, and T. Maniatis. 1989. Molecular cloning. A laboratory manual. Cold Spring Harbor, N.Y.: Cold Spring Harbor Laboratory Press.
Saruul, P., F. Srienc, D. A. Somers, and D. A. Samac. 2002. Production of a Biodegradable Plastic Polymer, Poly-β-Hydroxybutyrate, in Transgenic Alfalfa. Crop. Sci. 42(3): 919-927.
Schillberg, Fischer, and Emans. 2003. Molecular farming of recombinant antibodies in plants. Cell. Mol. Life. Sci. 60(3): 433-445.
Schillberg S., S. Zimmermann, A. Voss, and R. Fischer. 1999. Apoplastic and cytosolic expression of full-size antibodies and antibody fragments in Nicotiana tabacum. Trans. Res. 8(4): 255-263.
Schulman, A. H. 2002. Transgenic plants as producers of modified starch and other carbohydrates. In Plant Biotechnology and Transgenic Plants, ed. K. M. Oksman-Caldenetey and W. H. Barz, 255-282. New York: Basel.
Shapouri, H., J. A. Duffield, and M. Wang. 2002. The Energy Balance of Corn Ethanol: An Update. U.S. Dept. of Agriculture.
Sheldon, C. C., J. E. Burn, P. P. Perez, J. Metzger, J. A. Edwards, W. J. Peacock, and E. S. Dennis. The FLF MADS Box Gene: A Repressor of Flowering in Arabidopsis Regulated by Vernalization and Methylation. Plant Cell 11(3): 445-458.
Singh, S. P., E. Ekanem, T. Wakefield Jr., and S. Corner. 2003. Emerging Importance of Bio-Based Products and Bio-Energy in the U.S. Economy: Information Dissemination and Training of Students. Int. Food Agribus. Mgmt. Rev. 5(3): 1-15.
Smidansky E. D., J. M. Martin, C. L. Hannah, A. M. Fischer, and M. J. Giroux. 2003. Seed yield and plant biomass increases in rice are conferred by deregulation of endosperm ADP-glucose pyrophosphorylase. Planta 216(4): 656-664.
Sticklen, M. B. 2004. Production of microbial hydrolysis enzymes in biomass crops via genetic engineering. In: Proceedings of the 2nd International Ukrainian Conference on Biomass for Energy. Sep. 20-22, 2004. Kyiv, Ukraine. Ukraine Natl. Acad. Sci. Press. pp. 133-136.
Sticklen, M. B. 2006. Plant genetic engineering to improve biomass characteristics for biofuels. Curr. Opin. Biotech. 17(3): 315-319.
Sticklen, M. B. 2007a. Feedstock Crop Genetic Engineering for Alcohol Fuels. Crop Sci.: in review.
______. 2007b. Role of Transgenic Biomass Crops in Ethanol Refineries. J. Biobased Mater. Bioenergy: xx-xx in review.
Teymouri, F., H. Alizadeh, L. Laureano-Perez, B. E. Dale, and M. B. Sticklen. 2004. Effects of Ammonia Fiber Explosion Treatment on Activity of Endoglucanase from Acidothermus cellulolyticus in Transgenic Plant. Appl. Biochem. Biotech. 116:1183-1192.
Thurston, C. F. 1994. The structure and function of fungal laccases. Microbiol. 140:19-26.
Tucker, M. P., A. Mohagheghi, K. Grohmann, and M. E. Himmel. 1989. Ultra-Thermostable Cellulases From Acidothermus cellulolyticus: Comparison of Temperature Optima with Previously Reported Cellulases. Bio/Technol. 7(8): 817-820.
U.S. Prime Supplier Sales Volumes of Petroleum Products. http://tonto. eia. doe. gov/dnav/pet/pet_cons_prim_dcu_nus_a.htm (accessed Aug. 5, 2007).
Vain, P., M. D. McMullen, and J. J. Finer. 1993. Osmotic treatment enhances particle bombardment-mediated transient and stable transformation of maize. Plant Cell Rep. 12:84-88.
Warren, R. A. J. 1996. Microbial Hydrolysis of Polysaccharides. Annu. Rev. Microbiol. 50(1): 183-212.
World of Corn 2007. http://www.ncga.com/WorldOfCorn/main/consumption1.asp (accessed Aug. 5, 2007).
World of Corn 2007. http://www.ncga.com/WorldOfCorn/main/production1.asp (accessed Aug. 5, 2007).
Wyman, C. E. 1999. Biomass ethanol: Technical progress, opportunities, and commercial challenges. Annu. Rev. Energy Environ. 24:189-226.
Wyman, C. E., B. E. Dale, R. T. Elander, M. Holtzapple, M. R. Ladisch, and Y. Y. Lee. 2005a. Comparative sugar recovery data from laboratory scale application of leading pretreatment technologies to corn stover. Biores. Tech. 96(18): 2026-2032.
______. 2005b. Coordinated development of leading biomass pretreatment technologies. Biores. Tech. 96(18): 1959-1966.
Xu, D., X. Duan, B. Wang, B. Hong, T. H. D. Ho, and R. Wu. 1996. Expression of a Late Embryogenesis Abundant Protein Gene, HVA1, from Barley Confers Tolerance to Water Deficit and Salt Stress in Transgenic Rice. Plant Physiol. 110(1): 249-257.
Yang P., Y. Wang, Y. Bai, K. Meng, H. Luo, T. Yuan, Y. Fan, and B. Yao. 2007. Expression of xylanase with high specific activity from Streptomyces olivaceoviridis A1 in transgenic potato plants (Solanum tuberosum L.). Biotech. Lett. 29(4): 659-667.
Yao, J. Q. 2004. Genetic transformation of tobacco with a beta-glucosidase gene to induce constitutive systemic acquired resistance against tobacco mosaic virus. Western Michigan University, Kalamazoo, Mich.
Zhang, Y. H. P., and L. R. Lynd. 2006. A functionally based model for hydrolysis of cellulose by fungal cellulas. Biotechnol. Bioeng. 94(5): 888-898.
Zhong, H., F. Teymouri, B. Chapman, S. B. Maqbool, R. Sabzikar, Y. El-Maghraby, B. E. Dale, and M. B. Sticklen. 2003. The pea (Pisum sativum L.) rbcS transit peptide directs the Alcaligenes eutrophus polyhydroxybutyrate enzymes into the maize (Zea mays L.) chloroplasts. Plant Sci. 165(3): 455-462.
Ziegelhoffer, T., J. A. Raasch, and S. Austin-Phillips. 2001. Dramatic effects of truncation and sub-cellular targeting on the accumulation of recombinant microbial cellulase in tobacco. Mol. Breeding. 8(2): 147-158.
Ziegelhoffer T., J. Will, and S. Austin-Phillips. 1999. Expression of bacterial cellulase genes in transgenic alfalfa (Medicago sativa L.), potato (Solanum tuberosum L.) and tobacco (Nicotiana tabacum L.). Mol. Breeding. 5(4): 309-318.
Ziegler, M. T., S. R. Thomas, and K. J. Danna. 2000. Accumulation of a thermostable endo-1,4-β-D-glucanase in the apoplast of Arabidopsis thaliana leaves. Mol. Breeding. 6(1): 37-46.

We claim:

1. A transgenic monocot plant protein which degrades lignocellulose wherein the transgenic monocot plant is ground and extracted to produce the plant protein by a method comprising:
(a) providing in the monocot plant DNA or at least 80% homologous to the bglA gene, SEQ ID NO: 14 of Butyrivibrio fibrisolvens encoding a β-glucosidase which is operably linked to a nucleotide sequence encoding a signal peptide that directs the β-glucosidase to a subcellular compartment of the transgenic monocot plant;
(b) grinding the monocot plant after the β-glucosidase is accumulated; and
(c) extracting the plant protein from the ground monocot plant, optionally mixing the plant protein with another plant protein encoding a cellulase, and optionally mixing with a commercially available enzyme produced by a bacterium.

2. The transgenic monocot plant protein of claim 1 wherein the DNA encoding the signal peptide is operably linked to an endoplastic-reticulum leading sequence (ER) as the subcellular compartment.

3. The transgenic monocot plant protein of claim 2 wherein the promoter is CaMV 35S and the terminator is CaMV 35S-T.

4. The transgenic monocot plant protein of claim 1 wherein a second DNA encodes a cellulase is included.

5. The transgenic monocot plant protein of claim 1 wherein the Butyrivibrio fibrisolvens is from the rumen of a cow.

6. The transgenic monocot plant protein of claim 1 is from a plant selected from the group consisting of maize, wheat, barley, rye, hops, hemp, rice and grasses.

7. The transgenic monocot plant protein of claim 1 which is from maize.

8. The transgenic monocot plant protein of claim 1 wherein the transgenic monocot plant further includes a DNA encoding a selectable marker operably linked to a constitutive promoter.

9. The transgenic monocot plant protein of claim 8 wherein the DNA encoding the selectable marker provides the transgenic monocot plant with resistance to an antibiotic, an herbicide, or to environmental stress.

10. The transgenic monocot plant protein of claim 9 wherein the DNA encoding resistance to the herbicide is a DNA encoding phosphinothricin acetyl transferase which confers resistance to the herbicide phosphinothricin.

11. The transgenic monocot plant protein of claim 1 wherein the subcellular compartment is the apoplast of the plant.

12. A method for producing transgenic monocot plant proteins which degrade lignocellulose wherein the transgenic monocot plant is ground and extracted to produce the plant proteins comprising:
(a) providing a first transgenic monocot plant which includes the DNA or at least 80% homologous to bglA gene, SEQ ID NO: 1, of Butyrivibrio fibrisolvens encoding a β-glucosidase to a subcellular compartment of the transgenic monocot plant, and a second transgenic monocot plant encoding an enzyme other than the β-glucosidase which degrades cellulose;
(b) mating by sexual fertilization the first and the second transgenic monocot plants to produce a third transgenic monocot plant which includes the DNA encoding the β-glucosidase and the DNA encoding the enzyme other than β-glucosidase;
(c) grinding the monocot plant after the enzymes have accumulated; and
(d) extracting the plant proteins.

13. The method of claim 12 wherein the DNA encoding the cellulase and the DNA encoding the β-glucosidase are each operably linked to the signal peptide operatively linked to an endoplastic reticulum leading sequence (ER) as the subcellular compartment.

14. The method of claim 12 wherein the promoter is CaMV 35S and the terminator is CaMV 35S-T.

15. The method of claim 13 wherein the DNA encoding the β-glucosidase is a bglA gene of Butyrivibrio fibrisolvens.

16. The method of claim 12 wherein the plant is selected from the group consisting of maize, wheat, barley, rye, hops, rice and grasses.

17. The method of claim 12 wherein the transgenic monocot plant is maize.

18. The method of claim 12 wherein the first, second, or both transgenic monocot plants further include a DNA encoding a selectable marker operably linked to a constitutive promoter.

19. The method of claim 18 wherein the DNA encoding the selectable marker provides the transgenic monocot plant with resistance to an antibiotic, an herbicide, or to environmental stress.

20. The method of claim 19 wherein the DNA encoding resistance to the herbicide is a DNA encoding phosphinothricin acetyl transferase which confers resistance to the herbicide phosphinothricin.

21. The method of claim 12 wherein the subcellular compartment is apoplast endoplasmic reticulum vacuole, chloroplast or mitochondria of the plant.

22. The method of claim 12 wherein transformed progeny of the third transgenic monocot plant which includes the DNA encoding the β-glucosidase and the DNA encoding the enzyme other than β-glucosidase are mated by sexual fertilization to a transgenic monocot plant selected from the group consisting of the first, second, and third transgenic monocot plants to produce a transgenic monocot plant comprising different copies of the DNA encoding the β-glucosidase and different copies of the DNA encoding the cellulase.

23. A method for converting lignocellulose in a transgenic monocot plant material to fermentable sugars comprising:
(a) providing a transgenic monocot plant which includes the DNA or at least 80% homologous to bglA gene, SEQ ID NO: 14, of Butyrivibrio fibrisolvens encoding a β-glucosidase, which is operably linked to a nucleotide sequence encoding a signal peptide that directs the β-glucosidase to a subcellular compartment of the transgenic monocot plant;
(b) growing the transgenic monocot plant for a time sufficient for the transgenic monocot plant to accumulate a sufficient amount of the β-glucosidase in the subcellular compartment of the transgenic monocot plant;
(c) harvesting the transgenic monocot plant which has accumulated the β-glucosidase in the subcellular compartment of the transgenic monocot plant;
(d) grinding the transgenic monocot plant for a time sufficient to produce the transgenic monocot plant material wherein the β-glucosidase and transgenic monocot plant are released from the subcellular compartment;
(e) incubating the transgenic monocot plant material for a time sufficient for the β-glucosidase in the plant material to produce the fermentable sugars from the lignocellulose in the transgenic monocot plant material; and
(f) extracting the fermentable sugars from the transgenic monocot plant material.

24. The method of claim 23 wherein the DNA encoding the signal peptide is operably linked to an endoplastic reticulum leading sequence (ER) as the subcellular compartment.

25. The method of claim 24 wherein the promoter is CaMV 35S and the terminator is CaMV 35S-T.

26. The method of claim 23 wherein a second DNA encoding a cellulase is included.

27. The method of claim 25 or 26 wherein the Butyrivibrio fibrisolvens is from the rumen of a cow.

28. The method of claim 23 selected from the group consisting of maize, wheat, barley, rye, hops, hemp, rice and grasses.

29. The method of claim 23 which is maize.

30. The method of claim 23 wherein the transgenic monocot plant further includes a DNA encoding a selectable marker operably linked to a constitutive promoter.

31. The method of claim 30 wherein the DNA encoding the selectable marker provides the transgenic monocot plant with resistance to an antibiotic, an herbicide, or to environmental stress.

32. The method of claim 31 wherein the DNA encoding resistance to the herbicide is a DNA encoding phosphinothricin acetyl transferase which confers resistance to the herbicide phosphinothricin.

33. The method of claim 23 wherein the subcellular compartment is the apoplast of the plant.

34. The method claim of 23 further comprising adding to (d) a plant material from a non-transgenic monocot plant.

35. The method of claim 23 further comprising: the steps of fermenting the sugars in step (f) to ethanol.

36. The plant of claim 1 which has DNA encoding the flowering locus C (FLC) gene.

37. The method of claim 12 or 24 wherein the monocot plant has DNA encoding the flowering locus C (FLC) gene.

38. A transgenic monocot plant protein which degrades lignocellulose wherein the transgenic monocot plant is ground and extracted to produce the plant protein by a method comprising:
(a) providing in the monocot plant DNA encoding a xylanase having a DNA sequence of at least 80% homologous to XYL1 gene, SEQ ID NO: 16, which is operably linked to a nucleotide sequence encoding a signal peptide that directs the xylanase to a subcellular compartment of the transgenic monocot plant;
(b) grinding the monocot plant material after the xylanase has accumulated; and
(c) extracting the plant protein, optionally mixing the plant protein with another plant protein encoding a cellulase and optionally mixing with a commercially available enzyme produced by a bacterium.

39. The transgenic monocot plant protein of claim 38 wherein the DNA encoding the signal peptide is operably linked to an endoplastic-reticulum leading sequence (ER) as the subcellular compartment.

40. The transgenic monocot plant protein of claim 39 wherein the promoter is CaMV 35S or Rubisco rbcS, and the terminator is CaMV 35S-T.

41. The transgenic monocot plant protein of claim 38 wherein DNA encoding the xylanase is the XYL1 gene.

42. The transgenic monocot plant protein of claim 38 wherein the plant is selected from the group consisting of maize, wheat, barley, rye, hops, hemp, rice and grasses.

43. The transgenic monocot plant protein of claim 38 wherein the plant is maize.

44. The transgenic monocot plant protein of claim 38 wherein the transgenic monocot plant further includes a DNA encoding a selectable marker operably linked to a constitutive promoter.

45. The transgenic monocot plant protein of claim 44 wherein the DNA encoding the selectable marker provides the transgenic monocot plant with resistance to an antibiotic, an herbicide, or to environmental stress.

46. The transgenic monocot plant protein of claim 45 wherein the DNA encoding resistance to the herbicide is a DNA encoding phosphinothricin acetyl transferase which confers resistance to the herbicide phosphinothricin.

47. The transgenic monocot plant protein of claim 38 wherein the subcellular compartment is the apoplast of the plant.

48. A method for producing a transgenic monocot plant proteins which degrade lignocellulose wherein the transgenic monocot plant is ground and extracted to produce the plant proteins material comprising:
(a) providing a first transgenic monocot plant which includes the DNA encoding a xylanase having a DNA sequence at least 80% homologous to XYL1 gene, SEQ ID NO: 16, to a subcellular compartment of the transgenic monocot plant, and a second transgenic monocot plant encoding an enzyme other than the xylanase which degrades cellulose;
(b) mating by sexual fertilization the first and the second transgenic monocot plants to produce a third transgenic monocot plant which includes the DNA encoding the xylanase and the DNA encoding the enzyme other than xylanase;
(c) grinding the monocot plant after the enzymes have accumulated; and
(d) extracting the plant proteins.

49. The method of claim 48 wherein the DNA encoding the cellulase and the DNA encoding the xylanase are each operably linked to the signal peptide operatively linked to an endoplastic reticulum leading sequence (ER) as the subcellular compartment.

50. The method of claim 48 wherein the promoter is CaMV 35S and the terminator is CaMV 35S-T.

51. The method of claim 49 wherein the DNA encoding the xylanase is the XYL1 gene.

52. The method of claim 48 wherein the plant is selected from the group consisting of maize, wheat, barley, rye, hops, rice and grasses.

53. The method of claim 48 wherein the transgenic monocot plant is maize.

54. The method of claim 48 wherein the first, second, or both transgenic monocot plants further include a DNA encoding a selectable marker operably linked to a constitutive promoter.

55. The method of claim 54 wherein the DNA encoding the selectable marker provides the transgenic monocot plant with resistance to an antibiotic, an herbicide, or to environmental stress.

56. The method of claim 55 wherein the DNA encoding resistance to the herbicide is a DNA encoding phosphinothricin acetyl transferase which confers resistance to the herbicide phosphinothricin.

57. The method of claim 48 wherein the subcellular compartment is apoplast endoplasmic reticulum vacuole, chloroplast or mitochondria of the plant.

58. The method of claim 48 wherein transformed progeny of the third transgenic monocot plant which includes the DNA encoding the xylanase and the DNA encoding the enzyme other than xylanase are mated by sexual fertilization to a transgenic monocot plant selected from the group consisting of the first, second, and third transgenic monocot plants to produce a transgenic monocot plant comprising multiple copies of the DNA encoding the xylanase and multiple copies of the DNA encoding the enzyme other than the xylanase.

59. A method for converting lignocellulose in a transgenic monocot plant material to fermentable sugars comprising:
(a) providing a transgenic monocot plant which includes the DNA encoding a xylanase having a DNA sequence at least 80% homologous to XYL1 gene, SEQ ID NO: 16, which is operably linked to a nucleotide sequence encoding a signal peptide that directs the xylanase to a subcellular compartment of the transgenic monocot plant;
(b) growing the transgenic monocot plant for a time sufficient for the transgenic monocot plant to accumulate a sufficient amount of the xylanase in the subcellular compartment of the transgenic monocot plant;
(c) harvesting the transgenic monocot plant which has accumulated the xylanase in the subcellular compartment of the transgenic monocot plant;
(d) grinding the transgenic monocot plant to produce the transgenic monocot plant material, wherein the xylanase and transgenic monocot plant are released from the subcellular compartment;
(e) incubating the transgenic monocot plant material for a time sufficient for the xylanase in the plant material to produce the fermentable sugars from the lignocellulose in the transgenic monocot plant material; and
(f) extracting the fermentable sugars from the transgenic monocot plant material.

60. The method of claim 59 wherein the DNA encoding the signal peptide is operably linked to an endoplastic reticulum leading sequence (ER) as the subcellular compartment.

61. The method of claim 60 wherein the promoter is CaMV 35S and the terminator is CaMV 35S-T.

62. The method of claim 59 wherein DNA encoding the xylanase is the XYL1 gene.

63. The method of claim 59 selected from the group consisting of maize, wheat, barley, rye, hops, hemp, rice and grasses.

64. The method of claim 59 which is maize.

65. The method of claim 59 wherein the transgenic monocot plant further includes a DNA encoding a selectable marker operably linked to a constitutive promoter.

66. The method of claim 65 wherein the DNA encoding the selectable marker provides the transgenic monocot plant with resistance to an antibiotic, an herbicide, or to environmental stress.

67. The method of claim 66 wherein the DNA encoding resistance to the herbicide is a DNA encoding phosphinothricin acetyl transferase which confers resistance to the herbicide phosphinothricin.

68. The method of claim 59 wherein the subcellular compartment is the apoplast of the plant.

69. The method claim of 59 further comprising adding to (d) a plant material from a non-transgenic monocot plant.

70. The method of claim 59 further comprising: the steps of fermenting the sugars in step (f) to ethanol.

71. The plant of claim 38 which has DNA encoding the flowering locus C (FLC) gene.

72. The method of claim 48 or 60 wherein the monocot plant has DNA encoding the flowering locus C (FLC) gene.
